(19) United States
(12) Patent Application Publication
(10) Pub. No.: US 2025/0376633 A1
(43) Pub. Date: Dec. 11, 2025

(54) MICROBIAL COMPOSITIONS AND METHODS FOR HYDROGEN AND METHANE PRODUCTION

(71) Applicant: RAISON, LLC, Sioux Falls, SD (US)

(72) Inventors: Tony Hagen, Sioux Falls, SD (US); Cecil T. Massie, Bloomington, MN (US)

(21) Appl. No.: 18/871,969

(22) PCT Filed: Jun. 6, 2023
(86) PCT No.: PCT/US2023/024595

§ 371 Date: Dec. 5, 2024

Related U.S. Application Data
(60) Provisional application No. 63/349,377, filed on Jun. 6, 2022.

(51) Int. Cl. C10L 3/08; C12M 1/00; C12P 5/02; C12P 39/00

(52) U.S. Cl. CPC C10L 3/08; C12M 23/58; C12P 5/023; C12P 39/00; C10L 2290/26

(57) ABSTRACT

Disclosed herein are methods for selectively and separately producing hydrogen and methane using microbial compositions under anaerobic conditions to facilite the digestion of a biomass or landfill leachate.

### CROSS REFERENCE TO RELATED APPLICATIONS

This application claims the benefit of U.S. Provisional Application No. 63/349,377, filed Jun. 6, 2022. The content of this earlier filed application is hereby incorporated by reference herein in its entirety.

### FIELD

The present disclosure relates to compositions and methods for producing hydrogen and methane. The disclosure provides a microbial ensemble that can be used to maximize hydrogen and methane production in separate but connected reaction chambers.

### BACKGROUND

The need for energy is a constant issue in human society with the usage increasing annually. With the soaring energy demands and environmental pollution, improved and efficient and alternative methods to produce energy are needed.

### SUMMARY

The present disclosure relates to compositions comprising a Pseudomonas spp. and a Clostridium spp., and methods for using said compositions to selectively and separately produce hydrogen and methane.

Disclosed herein are methods for selectively and separately producing hydrogen and methane, the methods comprising: a) contacting a biomass in a first reactor vessel with a first microbial inoculant composition under anaerobic conditions to facilitate the digestion of the biomass to produce a digested biomass, wherein the first reactor vessel is maintained at a first oxidation reduction potential (ORP); b) collecting hydrogen gas from the first reactor vessel; c) transferring a portion of the digested biomass from step a) to a second reactor vessel; c) introducing an oxygen-containing gas to the second reactor vessel to change the first ORP from the first reactor vessel to a second ORP in the second reactor vessel and contacting the digested biomass in the second reactor vessel with a second microbial inoculant composition under anaerobic conditions to facilitate the digestion of the digested biomass; and d) collecting biogas from the second reactor vessel, wherein the first microbial inoculant comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2, wherein the second microbial inoculant comprises one or more methanogens selected from the group of consisting of Methanobacterium bryantii, Methanobacterium formicum, Methanobrevibacter arboriphilicus, Methanobrevibacter gottschalkii, Methanobrevibacter ruminantium, Methanobrevibacter smithii, Methanococcus chunghsingensis, Methanococcus burtonii, Methanococcus aeolicus, Methanococcus dellae, Methanococcus jannaschii, Methanococcus maripaludis, Methanococcus vannielii, Methanocorpusculum labreanum, Methanoculleus bourgensis (Methanogenium olentangyi and Methanogenium bourgense), Methanoculleus marisnigri, Methanoflorens stordalenmirensis, Methanofollis liminatans, Methanogenium cariaci, Methanogenium frigidum, Methanogenium organophilum, Methanogenium wolfei, Methanomicrobium mobile, Methanopyrus kandleri, Methanoregula boonei, Methanosaeta concilii, Methanosaeta thermophile, Methanosarcina acetivorans, Methanosarcina barkeri, Methanosarcina mazei, Methanosphaera stadtmanae, Methanospirillium hungatei, Methanothermobacter defluvii (Methanobacterium defluvii), Methanothermobacter thermautotrophicus (Methanobacterium thermoautotrophicum), Methanothermobacter thermoflexus (Methanobacterium thermoflexum), Methanothermobacter wolfei (Methanobacterium wolfei), and Methanothrix sochngenii.

Disclosed herein are methods for producing hydrogen, the methods comprising: a) contacting a biomass in a first reactor vessel with a first microbial inoculant composition under anaerobic conditions to facilitate the digestion of the biomass to produce a digested biomass, wherein the first reactor vessel is maintained at a first oxidation reduction potential (ORP); and b) collecting hydrogen gas from the first reactor vessel, wherein the first microbial inoculant comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2, wherein the second microbial inoculant comprises one or more methanogens selected from the group of consisting of Methanobacterium bryantii, Methanobacterium formicum, Methanobrevibacter arboriphilicus, Methanobrevibacter gottschalkii, Methanobrevibacter ruminantium, Methanobrevibacter smithii, Methanococcus chunghsingensis, Methanococcus burtonii, Methanococcus aeolicus, Methanococcus deltae, Methanococcus jannaschii, Methanococcus maripaludis, Methanococcus vannielii, Methanocorpusculum labreanum, Methanoculleus bourgensis (Methanogenium olentangyi and Methanogenium bourgense), Methanoculleus marisnigri, Methanoflorens stordalenmirensis, Methanofollis liminatans, Methanogenium cariaci, Methanogenium frigidum, Methanogenium organophilum, Methanogenium wolfei, Methanomicrobium mobile, Methanopyrus kandleri, Methanoregula boonei, Methanosaeta concilii, Methanosaeta thermophile, Methanosarcina acetivorans, Methanosarcina barkeri, Methanosarcina mazei, Methanosphaera stadimanae, Methanospirillium hungatei, Methanothermobacter defluvii (Methanobacterium defluvii), Methanothermobacter thermautotrophicus (Methanobacterium thermoautotrophicum), Methanothermobacter thermoflexus (Methanobacterium thermoflexum), Methanothermobacter wolfei (Methanobacterium wolfei), and Methanothrix sochngenii.

Disclosed herein are methods for selectively and separately producing hydrogen or methane, the methods comprising: a) contacting a biomass in a first reactor vessel with a first microbial inoculant composition under anaerobic conditions to facilitate the digestion of the biomass to produce a digested biomass, wherein the first reactor vessel is maintained at a first oxidation reduction potential (ORP); b) optionally collecting hydrogen gas from the first reactor vessel; c) transferring a portion of the digested biomass from step a) to a second reactor vessel; c) introducing an oxygen-containing gas to the second reactor vessel to change the first ORP from the first reactor vessel to a second ORP in the second reactor vessel and contacting the digested biomass in the second reactor vessel with a second microbial inoculant composition under anaerobic conditions to facilitate the digestion of the digested biomass; and d) collecting biogas from the second reactor vessel, wherein the first microbial inoculant comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2, wherein the second microbial inoculant comprises one or more methanogens selected from the group of consisting of Methanobacterium bryantii, Methanobacterium formicum, Methanobrevibacter arboriphilicus, Methanobrevibacter gottschalkii, Methanobrevibacter ruminantium, Methanobrevibacter smithii, Methanococcus chunghsingensis, Methanococcus burtonii, Methanococcus aeolicus, Methanococcus deltae, Methanococcus jannaschii, Methanococcus maripaludis, Methanococcus vannielii, Methanocorpusculum labreanum, Methanoculleus bourgensis (Methanogenium olentangyi and Methanogenium bourgense), Methanoculleus marisnigri, Methanoflorens stordalenmirensis, Methanofollis liminatans, Methanogenium cariaci, Methanogenium frigidum, Methanogenium organophilum, Methanogenium wolfei, Methanomicrobium mobile, Methanopyrus kandleri, Methanoregula boonei, Methanosaeta concilii, Methanosaeta thermophile, Methanosarcina acetivorans, mazei, Methanosphaera stadtmanae, Methanosarcina barkeri, Methanosarcina Methanospirillium hungatei, Methanothermobacter defluvii (Methanobacterium defluvii), Methanothermobacter thermautotrophicus (Methanobacterium thermoautotrophicum), Methanothermobacter thermoflexus (Methanobacterium thermoflexum), Methanothermobacter wolfei (Methanobacterium wolfei), and Methanothrix sochngenii.

Disclosed herein are methods for selectively and separately producing hydrogen and methane, the methods comprising: a) contacting a biomass in a first reactor vessel with a first microbial inoculant composition under anaerobic conditions to facilitate the digestion of the biomass to produce a digested biomass, wherein the first reactor vessel is maintained at a first oxidation reduction potential (ORP); b) collecting hydrogen gas from the first reactor vessel and transferring a portion of the digested biomass from step a) to a second reactor vessel; c) introducing an oxygen-containing gas to the second reactor vessel to change the first ORP from the first reactor vessel to a second ORP in the second reactor vessel and contacting the digested biomass in the second reactor vessel with a second microbial inoculant composition under aerobic conditions to facilitate the digestion of the digested biomass; and d) collecting biogas from the second reactor vessel, e) collecting a portion of the digested biomass from step a) and separating a liquid fraction from a solid fraction of the digested biomass, f) transferring the solid fraction of step e) into the first or second reactor vessel or both the first and second reactor vessels, g) transferring the liquid fraction or supernatant of step e) into a moving biofilm bed reactor (MBBR), contacting the liquid fraction in the MBBR with a microbial inoculant composition similar or the same as the content of the microbial inoculant composition used in the second reactor vessel; h) digesting the liquid fraction in the MBBR under conditions to remove one or more organic acids (e.g. acetate) from the liquid fraction to produce a liquid fraction with a reduced organic acid content; and i) optionally, transferring the liquid fraction or supernatant with a reduced organic acid content of step h) into the first reactor vessel, wherein the microbial inoculant comprising comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2, wherein the second microbial inoculant comprises one or more methanogens selected from the group of consisting of Methanobacterium bryantii, Methanobacterium formicum, Methanobrevibacter arboriphilicus, Methanobrevibacter gottschalkii, Methanobrevibacter ruminantium, Methanobrevibacter smithii, Methanococcus chunghsingensis, Methanococcus burtonii, Methanococcus aeolicus, Methanococcus dellae, Methanococcus jannaschii, Methanococcus maripaludis, Methanococcus vannielii, Methanocorpusculum labreanum, Methanoculleus bourgensis (Methanogenium olentangyi and Methanogenium bourgense), Methanoculleus marisnigri, Methanoflorens stordalenmirensis, Methanofollis liminatans, Methanogenium cariaci, Methanogenium frigidum, Methanogenium organophilum, Methanogenium wolfei, Methanomicrobium mobile, Methanopyrus kandleri, Methanoregula boonei, Methanosaeta concilii, Methanosaeta thermophile, Methanosarcina acetivorans, Methanosarcina barkeri, Methanosarcina mazei, Methanosphaera stadtmanae, Methanospirillium hungatei, Methanothermobacter defluvii (Methanobacterium defluvii), Methanothermobacter thermautotrophicus (Methanobacterium thermoautotrophicum), Methanothermobacter thermoflexus (Methanobacterium thermoflexum), Methanothermobacter wolfei (Methanobacterium wolfei), and Methanothrix sochngenii.

Disclosed herein are methods for selectively producing hydrogen from a landfill leachate, the methods comprising the steps of: a) applying a composition comprising two or more bacterial strains, wherein a first bacterial strain comprises Clostridium spp., and wherein the 16S sequence of Clostridium spp. comprises any one of the Clostridium spp. listed in Table 1 or Table 2 and a second bacterial strain comprising an aquatic Pseudomonas spp. bacteria with a 16S nucleic acid sequence that is at least about 97% identical to any one of the Pseudomonas spp. bacteria listed in Table 1 or Table 2 to the landfill leachate; b) collecting samples from the landfill leachate; c) introducing the landfill leachate sample into a first reactor vessel and contacting the landfill leachate sample with the microbial inoculant composition in step a) under anaerobic conditions to facilitate the digestion of the landfill leachate sample wherein the first reactor vessel is maintained at a first oxidation reduction potential (ORP); d) collecting hydrogen gas from the first reactor vessel and transferring a portion of the digested landfill leachate sample from step c) to a second reactor vessel; e) introducing an oxygen-containing gas to the second reactor vessel to change the first ORP from the first reactor vessel to a second ORP in the second reactor vessel and contacting the digested landfill leachate sample in the second reactor vessel with a second microbial inoculant composition under aerobic conditions to facilitate the digestion of the landfill leachate sample; f) collecting biogas from the second reactor vessel; g) collecting a portion of the digested landfill leachate sample from step c) and separating a liquid fraction from a solid fraction of the portion of the digested landfill leachate sample; h) transferring a portion of the solid fraction of step g) into the first or second reactor vessel or both the first and second reactor vessels; i) transferring the liquid fraction or supernatant of step g) into a moving biofilm bed reactor (MBBR), contacting the liquid fraction in the MBBR with a microbial inoculant composition similar or the same as the content of the microbial inoculant composition used in the second reactor vessel; j) digesting the liquid fraction in the MBBR under conditions to remove acetate from the liquid fraction to produce a liquid fraction with a reduced acetate content; k) optionally, transferring the liquid fraction or supernatant with a reduced acetate content of step j) into the first reactor vessel, wherein the first microbial inoculant comprising comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2, wherein the second microbial inoculant comprises one or more methanogens selected from the group of consisting of Methanobacterium bryantii, Methanobacterium formicum, Methanobrevibacter arboriphilicus, Methanobrevibacter gottschalkii, Methanobrevibacter ruminantium, Methanobrevibacter smithii, Methanococcus chunghsingensis, Methanococcus burtonii, Methanococcus aeolicus, Methanococcus deltae, Methanococcus jannaschii, Methanococcus maripaludis, Methanococcus vannielii, Methanocorpusculum labreanum, Methanoculleus bourgensis (Methanogenium olentangyi and Methanogenium bourgense), Methanoculleus marisnigri, Methanoflorens stordalenmirensis, Methanofollis liminatans, Methanogenium cariaci, Methanogenium frigidum, Methanogenium organophilum, Methanogenium wolfei, Methanomicrobium mobile, Methanopyrus kandleri, Methanoregula boonei, Methanosaeta concilii, Methanosaeta thermophile, Methanosarcina acetivorans, Methanosarcina barkeri, Methanosarcina mazei, Methanosphaera stadtmanae, Methanospirillium hungatei, Methanothermobacter defluvii (Methanobacterium defluvii), Methanothermobacter thermautotrophicus (Methanobacterium thermoautotrophicum), Methanothermobacter thermoflexus (Methanobacterium thermoflexum), Methanothermobacter wolfei (Methanobacterium wolfei), and Methanothrix sochngenii.

### BRIEF DESCRIPTION OF THE DRAWINGS

FIG. 1 shows an example of four steps of anaerobic digestion that can be employed in the disclosed methods.

FIG. 2 shows an example of anaerobic digestion chemistry that can be employed in the disclosed methods.

FIG. 3 shows an example of the workflow that can be employed in embodiments of the disclosed methods. As shown, exemplified is a stream flowing through a moving biofilm bed reactor (MBBR) from hydrogen forming to methane forming and then back to hydrogen forming when it re-enters the hydrogen reactor.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “comprising” can include the aspects “consisting of” and “consisting essentially of.”

As used herein the terms “microorganism” or “microbe” are used interchangeably and include, but are not limited to, the two prokaryotic domains, Bacteria and Archaea, eukaryotic fungi and protozoa, as well as viruses. In some aspects, the disclosure refers to the “microbes” of Table 1, Table 2, and/or Table 3 or the “microbes” incorporated by reference. This characterization can refer to not only the predicted taxonomic microbial identifiers of the Tables, but also the identified strains of the microbes listed in the Tables.

The term “microbial consortia” or “microbial consortium” refers to a subset of a microbial community of individual microbial species, or strains of a species, which can be described as carrying out a common function, or can be described as participating in, or leading to, or correlating with, a recognizable parameter or plant phenotypic trait. The community may comprise two or more species, or strains of a species, of microbes. In some instances, the microbes coexist within the community symbiotically.

The term “microbial community” means a group of microbes comprising two or more species or strains. Unlike microbial ensemble, a microbial community does not have to be carrying out a common function, or does not have to be participating in, or leading to, or correlating with, a recognizable parameter, such as a phenotypic trait of interest (e.g., increased amount of hydrogen in the rumen in a ruminant).

As used herein, “isolate,” “isolated,” “isolated microbe,” and like terms, are intended to mean that the one or more microorganisms has been separated from at least one of the materials with which it is associated in a particular environment (for example soil, water, animal tissue).

Thus, an “isolated microbe” does not exist in its naturally occurring environment; rather, it is through the various techniques described herein that the microbe has been removed from its natural setting and placed into a non-naturally occurring state of existence. Thus, the isolated strain or isolated microbe may exist as, for example, a biologically pure culture, or as spores (or other forms of the strain) in association with an acceptable carrier.

As used herein, “microbial composition” refers to a composition comprising one or more microbes of the present disclosure. For example, a “microbial composition” as used herein can comprise one or more of the microbes disclosed herein.

As used herein, “carrier”, “acceptable carrier”, or “pharmaceutical carrier” refers to a diluent, adjuvant, excipient, or vehicle with which the compound is administered. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are preferably employed as carriers, in some embodiments as injectable solutions. In some embodiments, gelling agents are employed as carriers. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration and standard pharmaceutical practice. See Hardee and Baggo (1998. Development and Formulation of Veterinary Dosage Forms. 2nd Ed. CRC Press. 504 pg.); E. W. Martin (1970. Remington's Pharmaceutical Sciences. 17th Ed. Mack Pub. Co.); and Blaser et al. (US Publication US20110280840A1).

The term “bioensemble,” “microbial ensemble,” or “synthetic ensemble” refers to a composition comprising one or more active microbes identified by methods, systems, and/or apparatuses of the present disclosure and that do not naturally exist in a naturally occurring environment and/or at ratios or amounts that do not exist in nature. A bioensemble is a subset of a microbial community of individual microbial species, or strains of a species, which can be described as carrying out a common function, or can be described as participating in, or leading to, or correlating with, a recognizable parameter, such as a phenotypic trait of interest (e.g. increased feed efficiency in feedlot cattle). The bioensemble may comprise two or more species, or strains of a species, of microbes. In some instances, the microbes coexist within the community symbiotically.

As used herein, “microbiome” refers to a collection of microorganisms that inhabit the digestive tract or gastrointestinal tract of an animal (including the rumen if said animal is a ruminant) and the microorganism's physical environment (i.e. the microbiome has a biotic and physical component). The microbiome can be fluid and may be modulated by numerous naturally occurring and artificial conditions (e.g., change in diet, disease, antimicrobial agents, influx of additional microorganisms, etc.). The modulation of the microbiome of a rumen that can be achieved via administration of the compositions of the disclosure, can take the form of: (a) increasing or decreasing a particular Family, Genus, Species, or functional grouping of microbe (i.e., alteration of the biotic component of the rumen microbiome) and/or (b) increasing or decreasing volatile fatty acids in the rumen, increasing or decreasing rumen pH, increasing or decreasing any other physical parameter important for rumen health (i.e., alteration of the abiotic component of the rumen microbiome).

The term “growth medium” as used herein, is any medium which is suitable to support growth of a microbe. By way of example, the media may be natural or artificial including gastrin supplemental agar, LB media, blood serum, and tissue culture gels. It should be appreciated that the media may be used alone or in combination with one or more other media. It may also be used with or without the addition of exogenous nutrients.

As used herein, “improved” should be taken broadly to encompass improvement of a characteristic of interest, as compared to a control group, or as compared to a known average quantity associated with the characteristic in question. For example, “improved” feed efficiency associated with application of a beneficial microbe, or microbial ensemble, of the disclosure can be demonstrated by comparing the feed efficiency of beef cattle treated by the microbes or feedstock treated with the disclosed microbes taught herein to the feed efficiency of beef cattle not treated by the microbes or feedstock treated with the disclosed microbes. In the present disclosure, “improved” does not necessarily demand that the data be statistically significant (i.e. p<0.05); rather, any quantifiable difference demonstrating that one value (e.g. the average treatment value) is different from another (e.g. the average control value) can rise to the level of “improved.” In some aspects, for an “improved” bioreactor production, lowering the pH below 6 can begin acidification of the media. In some aspects, the ORP can begin above 500 mV with the advent of acidosis, it will begin to fall (e.g., hydrogen production can begin with a reduction of about 50 mV). This, in turn, can cause a change in the population of microbes and the pH less than 6 and a mV reduction of 50 initiates the process, and the mV of ORP will drop to as low as about 600 mV during the process. In some aspects the maximization of hydrogen production can be observed in strata within the bioreactor, and the lowest ORP of about-50 mV with the lower ORP having the greatest production of hydrogen in the system. As used herein, “inhibiting and suppressing” and like terms should not be construed to require complete inhibition or suppression, although this may be desired in some embodiments.

The term “marker” or “unique marker” as used herein is an indicator of unique microorganism type, microorganism strain or activity of a microorganism strain. A marker can be measured in biological samples and includes without limitation, a nucleic acid-based marker such as a ribosomal RNA gene, a peptide- or protein-based marker, and/or a metabolite or other small molecule marker.

In the present disclosure, “nucleic acid” refers to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to naturally occurring nucleotides.

The terms “polypeptide,” “peptide,” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. Polypeptides of the present disclosure can be produced either from a nucleic acid disclosed herein, or by the use of standard molecular biology techniques. For example, a truncated protein of the present disclosure can be produced by expression of a recombinant nucleic acid of the embodiments in an appropriate host cell, or alternatively by a combination of ex vivo procedures, such as protease digestion and purification.

The term “encode” is used herein to mean that the nucleic acid comprises the required information, specified by the use of codons to direct translation of the nucleotide sequence into a specified protein. A nucleic acid encoding a protein can comprise non-translated sequences (e.g., introns) within translated regions of the nucleic acid or can lack such intervening non-translated sequences (e.g., as in cDNA).

Aspects of the disclosure encompass isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques (e.g. PCR amplification), or substantially free of chemical precursors or other chemicals when chemically synthesized. Optimally, an “isolated” polynucleotide is free of sequences (for example, protein encoding sequences) that naturally flank the polynucleotide (i.e., sequences located at the 5′ and 3′ ends of the polynucleotide) in the genomic DNA of the organism from which the polynucleotide is derived. For example, in some aspects of the disclosure, the isolated polynucleotide can contain less than about 5 kb, about 4 kb, about 3 kb, about 2 kb, about 1 kb, about 0.5 kb, or about 0.1 kb of nucleotide sequence that naturally flank the polynucleotide in genomic DNA of the cell from which the polynucleotide is derived. A protein that is substantially free of cellular material includes preparations of protein having less than about 30%, about 20%, about 10%, about 5%, or about 1% (by dry weight) of contaminating protein. When the protein of the aspects, or a biologically active portion thereof, is recombinantly produced, optimally culture medium represents less than about 30%, about 20%, about 10%, about 5%, or about 1% (by dry weight) of chemical precursors or non-protein-of-interest chemicals.

In some aspects, the term “substantially free of” can refer to a composition having less than about 1% by weight, e.g., less than about 0.5% by weight, less than about 0.1% by weight, less than about 0.05% by weight, or less than about 0.01% by weight of the stated material, based on the total weight of the composition. In some aspects, “substantially free of dissolved oxygen” can refer to an oxygen level in a bioreactor that is without any dissolved oxygen (e.g., about 0% dissolved oxygen) or with only a residual amount of dissolved oxygen remaining (e.g., no more than about 1%, no more than about 0.5%, no more than about 0.1%, no more than about 0.05%, or no more than about 0.01% dissolved oxygen).

The polynucleotides described herewith can be used to isolate corresponding sequences from other organisms, particularly other plants. In this manner, methods such as PCR or hybridization can be used to identify such sequences based on their sequence homology to the sequences set forth herein. Sequences isolated based on their sequence identity to the entire sequences set forth herein or to variants and fragments thereof are encompassed by the present disclosure. Such sequences include sequences that are orthologs of the disclosed sequences. The term “orthologs” refers to genes derived from a common ancestral gene and which are found in different species as a result of speciation. Genes found in different species are considered orthologs when their nucleotide sequences and/or their encoded protein sequences share at least about 60%, about 70%, about 75%, about 80%, about 85%, about 90%, about 91%, about 92%, about 93%, about 94%, about 95%, about 96%, about 97%, about 98%, about 99%, or greater sequence identity. Functions of orthologs are often highly conserved among species. Thus, isolated polynucleotides that encode for a protein that confers or enhances fungal plant pathogen resistance and that hybridize to the sequences disclosed herein, or to variants or fragments thereof, are encompassed by the present disclosure.

The terms “increase,” “increasing,” “enhance,” “enhancing” and the like are used herein to mean any boost or gain or rise in the amount of a composition (e.g., hydrogen). Further, the terms “induce” or “increase” as used herein can mean higher concentration of an amount of a composition (e.g., hydrogen), such that the level is increased 5% or more, 10% or more, 50% or more or 100% relative to a control subject or target.

The term “expression” as used herein in refers to the biosynthesis or process by which a polynucleotide, for example, is produced, including the transcription and/or translation of a gene product. For example, a polynucleotide of the present disclosure can be transcribed from a DNA template (such as into an mRNA or other RNA transcript) and/or the process by which a transcribed mRNA is subsequently translated into a polypeptide or protein. The term “gene product” can refer to for example, transcripts and encoded polypeptides. Inhibition of (or increase in) expression or function of a gene product (i.e., a gene product of interest) can be in the context of a comparison between any two plants, for example, expression or function of a gene product in a genetically altered plant versus the expression or function of that gene product in a corresponding, but susceptible wild-type plant or other susceptible plant. The expression level of a gene product in a wild-type plant can be absent.

Alternatively, inhibition of (or increase in) expression or function of the target gene product can be in the context of a comparison between plant cells, organelles, organs, tissues, or plant parts within the same plant or between plants, and includes comparisons between developmental or temporal stages within the same plant or between plants. Any method or composition that down-regulates expression of a target gene product, either at the level of transcription or translation, or down-regulates functional activity of the target gene product can be used to achieve inhibition of expression or function of the target gene product. Similarly, any method or composition that induces or up-regulates expression of a target gene product, either at the level of transcription or translation, or increases or activates or up-regulates functional activity of the target gene product can be used to achieve increased expression or function of the target gene or protein. Methods for inhibiting or enhancing gene expression are well known in the art.

“Percentage of sequence identity”, as used herein, is determined by comparing two optimally locally aligned sequences over a comparison window defined by the length of the local alignment between the two sequences. The amino acid sequence in the comparison window may comprise additions or deletions (e.g., gaps or overhangs) as compared to the reference sequence (which does not comprise additions or deletions) for optimal alignment of the two sequences. Local alignment between two sequences only includes segments of each sequence that are deemed to be sufficiently similar according to a criterion that depends on the algorithm used to perform the alignment (e. g. BLAST). The percentage of sequence identity is calculated by determining the number of positions at which the identical nucleic acid base or amino acid residue occurs in both sequences to yield the number of matched positions, dividing the number of matched positions by the total number of positions in the window of comparison and multiplying the result by 100. Optimal alignment of sequences for comparison may be conducted by the local homology algorithm of Smith and Waterman (Add. APL. Math. 2:482, 1981), by the global homology alignment algorithm of Needleman and Wunsch (J Mol. Biol. 48:443, 1970), by the search for similarity method of Pearson and Lipman (Proc. Natl. Acad. Sci. USA 85:2444, 1988), by heuristic implementations of these algorithms (NCBI BLAST, WU-BLAST, BLAT, SIM, BLASTZ), or by inspection. Given that two sequences have been identified for comparison, GAP and BESTFIT are preferably employed to determine their optimal alignment. Typically, the default values of 5.00 for gap weight and 0.30 for gap weight length are used. The term “substantial sequence identity” between polynucleotide or polypeptide sequences refers to polynucleotide or polypeptide comprising a sequence that has at least 50% sequence identity, preferably at least 70%, preferably at least 80%>, preferably at least 85%, preferably at least 90%>, preferably at least 95%, and preferably at least 96%>, 97%, 98% or 99% sequence identity compared to a reference sequence using the programs. In addition, pairwise sequence homology or sequence similarity, as used, refers to the percentage of residues that are similar between two sequences aligned. Families of amino acid residues having similar side chains have been well defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Query nucleic acid and amino acid sequences can be searched against subject nucleic acid or amino acid sequences residing in public or proprietary databases. Such searches can be done using the National Center for Biotechnology Information Basic Local Alignment Search Tool (NCBI BLAST v 2.18) program. The NCBI BLAST program is available on the internet from the National Center for Biotechnology Information (blast.ncbi.nlm.nih.gov/Blast.cgi). Typically the following parameters for NCBI BLAST can be used: Filter options set to “default”, the Comparison Matrix set to “BLOSUM62”, the Gap Costs set to “Existence: 11, Extension: 1”, the Word Size set to 3, the Expect (E threshold) set to 1e-3, and the minimum length of the local alignment set to 50% of the query sequence length. Sequence identity and similarity may also be determined using GenomeQuest™ software (Gene-IT, Worcester Mass. USA).

“Inoculant” as used herein refers to any culture or preparation that comprises at least one microorganism. In some aspects, an inoculant (sometimes as microbial inoculant, or soil inoculant) is an agricultural amendment that uses beneficial microbes (including, but not limited to endophytes) to promote plant health, growth and/or yield, animal health, growth or improvement of one or more traits. Many of the microbes suitable for use in an inoculant form symbiotic relationships with the target crops where both parties benefit (mutualism).

A “bioreactor,” “reactor vessel” or bioreactor vessel” as used herein refers to any device or system that supports a biologically active environment. As described herein a bioreactor can be a vessel in which microorganism(s) including the microorganism(s) disclosed herein can be grown or introduced. In some aspects, one or more of the reactor vessels disclosed herein can be continuous or discontinuous with one or more additional reactor vessels. In some aspects, one or more of the reactor vessels can be washed out prior to the addition of a biomass or prior to microorganisms being contacted or transferred into said reactor vessel.

As used herein, the phrase “hydrogen producing microorganisms” means microorganisms capable of fermenting organics under anaerobic conditions to produce hydrogen, carbon dioxide, and a variety of organic acids and alcohols. Examples of hydrogen generating microorganisms include, but are not limited to bacteria from the genera: Clostridium, Enterobacter, Klebsiella, Citrobacter, and Bacillus. For example, examples of hydrogen generating microorganisms include, but are not limited to, C. acetobutyricum, Bacillus thuringiensis, C. butyricum, C. saccharolyticum, and C. saccharobutylicum.

As used herein, the phrase “organic waste” refers to wastes that include carbon and hydrogen such as, but are not limited to, alcohols, ketones aldehydes, volatile fatty acids, esters, carboxylic acids, ethers, carbohydrates, proteins, lipids, polysaccharides, monosaccharide, cellulose, and nucleic acids. Examples of organic waste include but are not limited to green waste, food waste, food-soiled paper, non-hazardous wood waste, and landscape and pruning waste. In some aspects, organic waste can be any material that comes from a plant or an animal and is biodegradable. In some aspects, organic waste can be manure. The manure can be from any mammal or any animal (e.g., any livestock animal). For example, the manure can be from a human, a cow, a hog, a pig, a horse, a goat, a sheep, a buffalo, a donkey, a camel, a yak, a mule, or a boar.

As used herein, the term “methanogens” or “methanogen producers” refers to microorganisms that are capable of methane production under anaerobic conditions. As used herein, the term “methanogens” or “methanogen producers” can include coccoid (spherical shaped) or bacilli (rod shaped). There are over 50 described species of methanogens, which do not form a monophyletic group (since haloarchaea emerged from within them), although all known methanogens belong to Euryarchaeota. They are mostly anaerobic organisms that cannot function under aerobic conditions, but recently a species (Candidatus Methanothrix paradoxum) has been identified that can function in anoxic microsites within aerobic environments and is therefore also a “methanogen” or “methanogen producer” as used herein. Examples of methanogens as used herein include, but are not limited to, Methanobacterium bryantii, Methanobacterium formicum, Methanobrevibacter arboriphilicus, Methanobrevibacter gottschalkii, Methanobrevibacter ruminantium, Methanobrevibacter smithii, Methanococcus chunghsingensis, Methanococcus burtonii, Methanococcus aeolicus, Methanococcus deltae, Methanococcus jannaschii, Methanococcus maripaludis, Methanococcus vannielii, Methanocorpusculum labreanum, Methanoculleus bourgensis (Methanogenium olentangyi and Methanogenium bourgense), Methanoculleus marisnigri, stordalenmirensis, Methanofollis liminatans, Methanogenium cariaci, Methanoflorens Methanogenium frigidum, Methanogenium organophilum, Methanogenium wolfei, Methanomicrobium mobile, Methanopyrus kandleri, Methanoregula boonei, Methanosaeta concilii, Methanosaeta thermophile, Methanosarcina acetivorans, Methanosarcina barkeri, Methanosarcina mazei, Methanosphaera stadtmanae, Methanospirillium hungatei, Methanothermobacter defluvii (Methanobacterium defluvii), Methanothermobacter thermautotrophicus (Methanobacterium thermoautotrophicum), Methanothermobacter thermoflexus (Methanobacterium thermoflexum), Methanothermobacter wolfei (Methanobacterium wolfei), and Methanothrix sochngenii.

As used herein, the term “biomass” or “biomass feedstock” refers to any biological material, mixture, combination, derivative, or residual thereof that can be anaerobically digested to produce hydrogen and methane. Biomass feedstock may include, but is not limited to carbonaceous material such as plant material, plant waste (e.g., agricultural waste or crop waste), animal material, food waste, industrial waste, and organic waste products and residue thereof. In some aspects, the biomass or the biomass feedstock can be sterile or non-sterile. In some aspects, the the biomass or the biomass feedstock can be pretreated or non-pretreated.

As used herein, “residence time” refers to the mean time a volume of liquid or solid remains in the reactor volume. In some aspects, for a batch process, the residence time can be the batch cycle time. In some aspects, for a continuously fed reactor operating at steady state in continuous overflow, the mean residence time can be the reactor volume divided by volumetric flow rate.

Disclosed herein are reactor vessel design strategies and construction of an anaerobic reactor vessel digest process that can be used to produce hydrogen and methane in separate but connected reaction chambers (e.g., reactor vessels). The methods disclosed herein comprise using the disclosed reactor vessel design to maximize hydrogen recovery while optimizing the combined energy recovery of hydrogen and methane. In some aspects, the methods can comprise an optional final process step to convert ammonia to nitrates to reduce fugitive ammonia released to the atmosphere.

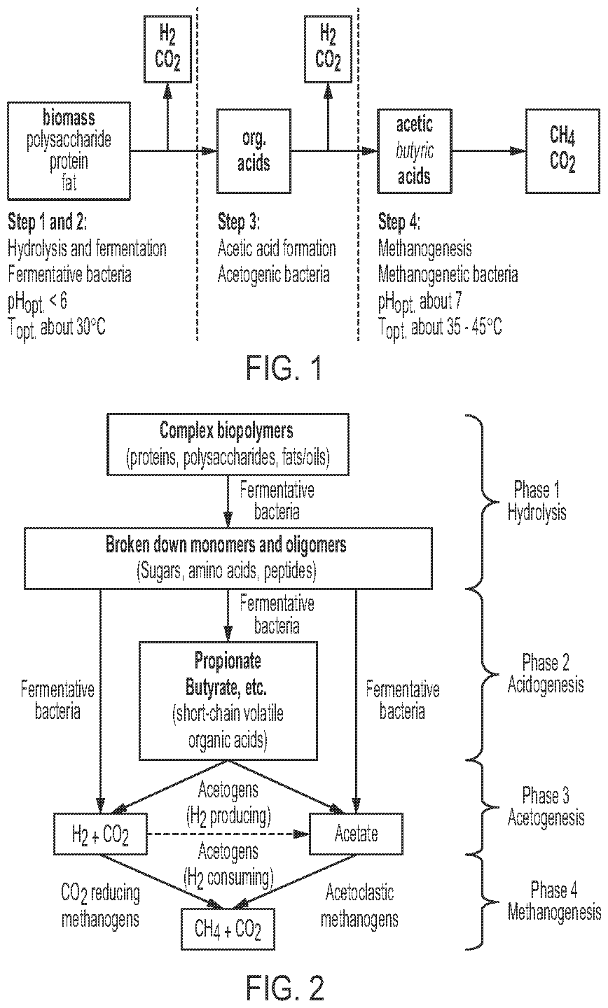

Disclosed herein are methods of producing hydrogen and methane separately during anaerobic fermentation. In some aspects, the methods can be used to achieve “selective sterilization” by biological means. As disclosed herein, methanogens can be selectively killed in the first three stages while leaving hydrogen formers and hydrogen forming acetogens to grow. To confirm that the process is working is to measure and regulate the Oxidation Reduction Potential (ORP) (e.g., which can reach-400 mV). Methanogens are most comfortable around-300 mV and are unable to reproduce at −400 mV. FIG. 1 shows the fours steps of anaerobic digestion.

As used herein, “oxidation-reduction potential”, or “ORP”, refers to a measurement that indicates the degree to which a substance is capable of oxidizing or reducing another substance. ORP is measured in millivolts (mV) using an ORP meter.

The methods disclosed herein can be used to maximize hydrogen production by facilitating the growth of favorable organisms in steps 1, 2 and 3 in a first reactor (as shown in FIG. 1) and simultaneously suppressing the growth of methanogens. Then, when hydrogen production is optimized, methanogens can be reintroduced in a second reactor with enough oxygen to raise the ORP to −300 to maximize total energy yield. FIG. 2 shows the overall chemistry of anaerobic digestion, and, in particular, the consumption of the waste products made during hydrogen production by methanogens. When methanogens and hydrogen formers coexist, most if not all, of the hydrogen formed can be consumed to make methane. In some aspects, an increase in ORP of at least about 50 mV can imitate the process.

The reactor vessel design and methods of using disclosed herein have several applications. In some aspects, reactor vessel design and methods can be applied to feeding program manures. This reactor design strategy can be used as part of of feeding program for dairy or other animals. In some aspects, nutrition programs can be employed to increase feed efficiency by suppression of methanogens in the rumen. The significance to this digester design is that there are few if any methanogens in the cow's manure. Thus, the organisms needed to continue breaking down complex organics to form short chain organic acids and hydrogen are present, but methanogens are not. This means the manure from these cows is suited to co-produce hydrogen and methane.

In some aspects, reactor vessel design and methods can be applied to other manures and feedstocks. The application of this process strategy is not limited to manures from animals on any particular feeding program. Other anaerobic digester feedstocks such as food wastes or energy crops can benefit from this approach because the microbial inoculant disclosed herein when introduced to the first reactor vessel actively suppresses methanogens while promoting hydrogen production. This means that a feedstock which contains methanogens can be rendered methanogen free without killing the desired species. This “selective sterilization” allows the use of any feedstock in this process strategy.

The general design can use a first reactor vessel for hydrogen production and then a second reactor vessel for methane production. For entirely new installations, the relative size of the hydrogen and methane reactor vessels can reflect the much faster acid/hydrogen formation compared to the slower methanogenesis. In some aspects, the hydrogen reactor can be operated as a complete-mix, constant-overflow design or in plug flow. The methane reactor can be operated as a plug flow design but with added recycle to inoculate the methanogen free overflow from the hydrogen reactor and raise the ORP.

The reactor design disclosed herein can also be used where there is an existing digester system. For example, where there is an existing digester, the existing digester can be used as the methane reactor and a hydrogen reactor can be inserted into the process flow. It is recognized some reconfiguration of the plug flow reactor flow may be required to manage pH at the start of the reactor and inoculate the incoming overflow from the hydrogen reactor.

In some aspects, high solids digesters can be distinguished from conventional digesters because the feedstock is stackable and not flowable. The digester looks like a gas tight garage and the feedstock is stacked in the chamber like a bunker silo. In these digesters, liquid can be circulated through the stack and gas evolved can collected in the unoccupied volume of the chamber. This design can be set up as a two stage digestion system because leachate can be continuously removed from the reactor volume. This leachate can be enriched with short chain organic acids used by methanogens. Since the solids can be removed, the leachate can be used for moving biofilm bed reactors (MBBR).

A landfill is an example of high solids digester. When organic waste is dumped into a landfill, it can undergo anaerobic decomposition (due to the lack of oxygen), and produces methane. In some aspects, hydrogen forming bacteria can be injected into the landfill. Hydrogen can be collected from the bed with technology similar to that used to harvest landfill gas. Leachate circulated through the landfill cells will carry organic acids to the surface where they will be converted to biogas in an MBBR.

As disclosed herein, the reactor design can be used to maximize hydrogen production and recovery by separating the hydrogen forming bacteria cultures from the methanogens. Solids and higher molecular weight molecules can be first hydrolyzed to long chain organic acids. The reactions producing hydrogen from long chain acids can lead to long chain organic acids being converted to hydrogen and acetic acid. Note that each reaction results in two or more organic acids (e.g., acetate (CH₃COO⁻), pyruvate, carboxylic acid, acetic acid (vinegar), and the like) groups which will lower the pH.

As the conversion proceeds, the accumulating acetic acid lowers the pH until, at some point, the environment becomes hostile to the hydrogen forming bacteria and biological activity stops. In the cow's rumen, this condition can be avoided by absorption of acids into the blood stream. In a conventional anaerobic digester, the acids can be consumed by methanogens and the system remains in balance.

In a two-stage digester, however, organic acids, like acetate, can accumulate and ultimately stop the process. In some aspects, the organic acid inhibition does not occur until the long chain acids are converted to organic acids (e.g., acetate). In some aspects, some of the unhydrolyzed solids and some of the long chain organic acids will overflow to the methanogen reactor resulting in lost hydrogen production. Some of this lost hydrogen can be avoided by adding a third reactor vessel alongside the hydrogen reactor (e.g., the first reactor vessel). In some aspects, the third reactor vessel can be designed as a moving bed biofilm reactor (MBBR). The MBBR can be filled with media (e.g., as manufactured by Lenntech). Methanogens can grow on the surface of the media. Owing to the extraordinary surface area, this media allows a high methanogen population to occupy a small volume. The purpose of the MBBR is to mimic the functionality of the rumen by selectively removing acetate from the hydrogen reactor.

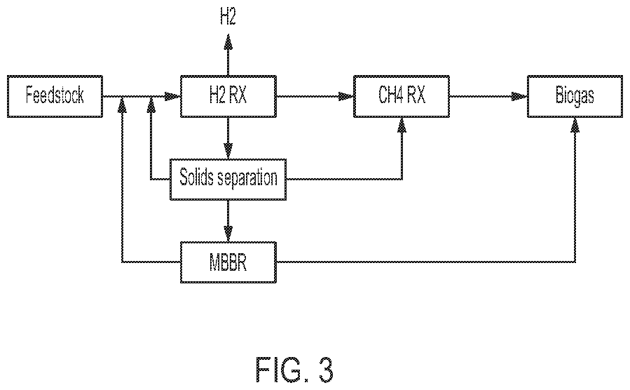

As disclosed herein, a portion of the hydrogen reactor volume can be be circulated through a liquid/solids separation to produce a supernatant containing acetate and long chain organic acids. Since the methanogens can be selective for acetate, the long chain acids will be unaffected and pass through the MBBR to be returned to the hydrogen reactor. Separated solids can be returned to the hydrogen reactor (e.g., first reactor vessel) or overflowed to the methanogen reactor (e.g., second reactor vessel). Recycle from the MBBR can be reduced in acetate content but retain long chain organic acids. Using this approach can maximize hydrogen production and methane production. This method can be carried out by swinging the ORP of the stream flowing through the MBBR from hydrogen forming to methane forming and then back to hydrogen forming when it re-enters the hydrogen reactor (also referred to as a ORP swing reactor) shown in FIG. 3.

### Compositions

Described herein are compositions (e.g. microbial inoculant compositions) comprising aquatic or primarily aquatically-derived microbial species for use in producing hydrogen, methane or a combination thereof. In some aspects, the microbial inoculant compositions comprises a species that produces and/or maintains a microenvironment that is suitable for other microbes in a microbial inoculant composition to thrive.

Disclosed herein are compositions (e.g. microbial inoculant compositions) comprising one or more of the microbes listed in Table 1, Table 2, Table 3 or Table 4. Disclosed herein are microbial inoculant compositions comprising one or more of the microbes listed in Table 1, Table 2, Table 3 or Table 4. Also disclosed herein are microbial inoculant compositions comprising one or more methanogens.

Disclosed herein are compositions (e.g., microbial inoculant compositions) comprising two or more bacterial strains. In some aspects, a first bacterial strain comprises Clostridium spp. In some aspects, the 16S sequence of Clostridium spp. comprises any one of the Clostridium spp. listed in Table 1 or Table 2. In some aspects, a second bacterial strain comprises an aquatic Pseudomonas spp. bacteria with a 16S nucleic acid sequence that is at least about 97% identical to any one of the Pseudomonas spp. bacteria listed in Table 1 or Table 2.

Disclosed herein are microbial inoculant compositions comprising one or more of the microbes listed in Table 1, Table 2, Table 3 or Table 4. In some aspects, the microbial inoculant compositions disclosed herein can further comprise one or more of the microbes listed in Table 1, Table 2, Table 3, or Table 4. In some aspects, the microbial inoculant compositions disclosed herein can further comprise at least one different microbial strain. In some aspects, the 16S sequence of the one different microbial strain can comprise a 16S sequence that is at least about 97% identical to one or more of the 16S sequences listed in Table 1, Table 2, Table 3, or Table 4.

In some aspects, the microbial inoculant compositions disclosed herein can further comprise an agriculturally effective amount of a compound or composition selected from the group consisting of a nutrient, a fertilizer, an acaricide, a bactericide, a fungicide, an insecticide, a microbicide, a nematicide, and a pesticide.

In some aspects, the microbial inoculant compositions disclosed herein can further comprise a carrier. In some aspects, the carrier can be peat, turf, talc, lignite, kaolinite, pyrophyllite, zeolite, montmorillonite, alginate, press mud, sawdust, perlite, mica, silicas, quartz powder, calcium bentonite, vermiculite or mixtures thereof.

In some aspects, the microbial inoculant compositions disclosed herein can be prepared as a formulation selected from the group consisting of an emulsion, a colloid, a dust, a granule, a pellet, a powder, a spray, and a solution.

In some aspects, the microbial inoculant compositions disclosed herein can comprise two or more bacterial strains, wherein a first bacterial strain comprises Clostridium spp., and wherein the 16S sequence of Clostridium spp. comprises any one of the Clostridium spp. listed in Table 1 or Table 2, a second bacterial strain comprising an aquatic Pseudomonas spp. bacteria with a 16S nucleic acid sequence that is at least about 97% identical to any one of the Pseudomonas spp. bacteria listed in Table 1 or Table 2 and one or more of the microbes listed in Table 1, Table 2 or Table 3. In some aspects, the microbial inoculant compositions disclosed herein can further comprise a composition that has at least one different microbial strain, wherein the 16S sequence of the one different microbial strain comprises a 16S sequence that is at least about 97% identical to one or more of the 16S sequences listed in Table 1, Table 2 or Table 3.

The primary structure of major rRNA subunit 16S comprise a particular combination of conserved, variable, and hypervariable regions that evolve at different rates and enable the resolution of both very ancient lineages such as domains, and more modern lineages such as genera. The secondary structure of the 16S subunit include approximately 50 helices which result in base pairing of about 67% of the residues. These highly conserved secondary structural features are of great functional importance and can be used to ensure positional homology in multiple sequence alignments and phylogenetic analysis. Over the previous few decades, the 16S rRNA gene has become the most sequenced taxonomic marker and is the cornerstone for the current systematic classification of bacteria and archaea (Yarza et al. 2014. Nature Rev. Micro. 12:635-45).

A sequence identity of 94.5% or lower for two 16S rRNA genes is strong evidence for distinct genera, 86.5% or lower is strong evidence for distinct families, 82% or lower is strong evidence for distinct orders, 78.5% is strong evidence for distinct classes, and 75% or lower is strong evidence for distinct phyla. The comparative analysis of 16S rRNA gene sequences enables the establishment of taxonomic thresholds that are useful not only for the classification of cultured microorganisms but also for the classification of the many environmental sequences. Yarza et al. 2014. Nature Rev. Micro. 12:635-45).

Microbial inoculant compositions can increase solubilization, uptake, and/or assimilation of nutrients such as, for example, carbon, nitrogen, potassium, phosphorus, selenium, cobalt, zinc, and copper.

Described herein are microbial inoculant compositions isolated from an aquatic environment for application to terrestrial plants. In some aspects, the inoculant mixture also comprises a species that produces and/or maintains a microenvironment in the plant that is suitable for other microbes in the inoculant mixture to thrive.

Described herein are microbial inoculant compositions that can include a Pseudomonas spp. and a Clostridium spp., such as, for example, P. fluorescens and C. saccharobutylicum.

In some aspects, a microbial inoculant composition further comprises one or more of Agrobacterium tumefaciens (TPD7005), Bacillus megaterium (TPD7007), Bacillus megaterium (TPD 7008), Agrobacterium rhizogenes (1713117 009), Microbacterium testaceum (TPD7010), Bacillus megaterium (TPD7011), Microbacterium spp. (TPD7012), Pedobacier kribbensis (TPD70013), Janthinobacterium lividum (TPD7014), Bacillus racemilacticus (TPD7015), Bacillus megaterium (TPD 7018), Delftia spp. (TPD3002), Chryseobacterium spp. (TPD3003), Bacillus licheniformis, Brevundimonas kwangchunensis (TPD3004), Fictibacillus barbaricus/Bacillus barbaricus (TPD3005), Prosthecobacter spp. (TPD3006), Lactobacillus plantarum (TPD3007), Sphingobacterium multivorum, Sphingomonas spp. (TPD3009), Sphingosinicella microcystinivorans (TPD3010), Pseudomonas chlororaphis, Pseudomonas mandelii, Pseudomonas umsongensis, Clostridium saccharobutylicum (TPD3014), Arthrobacter ramosus (TPD3015), Streptomyces yogyakartensis (TPD3016), Arthrobacter spp. (TPD3017), Xanthomonas spp., Chryseobacterium indologenes (TPD3019), or Lactobacillus plantarum.

Table 1 shows 16S RNA analysis and/or whole genome shotgun sequencing project data for exemplary members of an exemplary microbial inoculant composition.

TABLE 1
Microbes

| Species | Designation | GenBank Accession No. |
|---|---|---|
| Pseudomonas veronii | TPD3012 | MH190219.1 |
| Pseudomonas mandelii | TPD3013 | MH221124.1 |
| Pseudomonas mandelii | | CP005960.1 |
| Pseudomonas moraviensis | TPD3001 | MH190053.1 |
| Pseudomonas protegens | TPD3011 | MH221127.1 |
| Pantoea agglomerans | TPD7001 | MH190052.1 |
| Pantoea agglomerans | | CP016889.1 |
| Clostridium saccharobutylicum | TPD3014 | MH189851.1 |
| Clostridium saccharobutylicum | TPD7003 | MH192394.1 |
| Erwinia aphidicola | TPD7004 | MH190220.1 |
| Serratia liquefaciens | TPD7002 | MH190215.1 |
| Pedobacter kribbensis | TPD70013 | MH221086.1 |
| Janthinobacterium lividum | TPD7014 | MH221099.1 |
| Bacillus racemilacticus | TPD7015 | MH221098.1 |
| Sphingomonas spp. | TPD3009 | QDFK00000000.1 |
| Sphingomonas sp. | | CP015521.1 |
| Agrobacterium tumefaciens | TPD7005 | QDFL00000000.1 |
| Agrobacterium tumefaciens | | AE007869.2 |
| Bacillus megaterium | TPD7018 | QDFM00000000.1 |
| Sphingomonas spp. | TPD3009 | QDFN 0000000.1 |
| Bacillus megaterium | TPD7007 | QDFO00000000.1 |
| Bacillus megaterium | TPD7008 | QDFP00000000.1 |
| Bacillus megaterium | | CP001983.1 |
| Arthrobacter spp. | TPD3018 | QDFQ00000000.1 |
| Arthrobacter sp. | | CP022436.1 |
| Agrobacterium rhizogenes | TPD7009 | QDFR00000000.1 |
| Agrobacterium rhizogenes | | CP019701.1 |
| Sphingomonas melonis | TPD3008 | QDFS00000000.1 |
| Sphingomonas melonis | | CP023705.1 |
| Microbacterium testaceum | TPD7010 | QDFT00000000.1 |
| Microbacterium testaceum | | AP012052.1 |
| Bacillus megaterium | TPD7011 | QDFU00000000.1 |
| Microbacterium spp. | TPD7012 | QDFV00000000.1 |
| Microbacterium sp. | | AP017975.1 |

Table 2 shows bacterial strains that can be useful in the microbial inoculant compositions and methods disclosed herein.

TABLE 2
Microbes.

| Species | GenBank Accession No. |
|---|---|
| arthrobacter ramosus | CP022436.1 |
| arthrobacter sp. | CP022436.1 |
| brevundimonas kwangchunensis | |
| chryseobacterium sp. | |
| clostridium spp. | |
| clostridium uliginosum | |
| delftia spp. | |
| fictibacillus bacillus barbaricus | |
| lactobacillus plantarum | |
| prosthecobacter | |
| pseudomonas chlororaphis | |
| pseudomonas mandelii | CP005960.1 |
| pseudomonas spp. | LT707063.1 |
| pseudomonas umsongensis | |
| sphingobacterium multivorum | |
| sphingomonas sp. | CP015521.1 |
| sphingosinicella microcystinivorans | |
| streptomyces yogyakartansis | |

Table 3 shows bacterial strains that can be useful in the microbial inoculant compositions and methods disclosed herein.

TABLE 3
Microbes.

| Species | GenBank Accession No. |
|---|---|
| acetivibrio cellulolyticus | |
| acetobacteraceae | |
| acidimicrobiaceae | |
| acidimicrobiales | |
| acidimicrobium spp. | |
| acidiphilium | |
| aciditerrimonas | |
| aciditerrimonas sp. | |
| aciditerrimonas spp. | |
| acidobacteria | |
| acidobacteriaceae | |
| acidobacteriales | |
| acidobacteriia | |
| acidobacterium | |
| acidobacterium spp. | |
| acidovorax | |
| acidovorax citrulli | |
| acinetobacter lwoffii | |
| actinoallomurus iriomotensis | |
| actinobacteria | |
| actinomadura | |
| actinomadura sp. | |
| actinomyces | |
| actinomycetales | |
| actinopolymorpha | |
| actinopolymorpha pittospori | |
| actinotalea fermentans | |
| adhaeribacter spp. | |
| adhaeribacter terreus | |
| aeromicrobium fastidiosum | |
| aeromicrobium spp. | |
| afipia sp. | |
| afipia spp. | |
| agromyces subbeticus | |
| agromyces ulmi | |
| alcaligenaceae | |
| algoriphagus sp. | |
| alphaproteobacteria | |
| altererythrobacter alterierythrobacter sp. | |
| altererythrobacter sp. | |
| altererythrobacter spp. | |
| alteromonadaceae | |
| amaricoccus sp. | |
| aminobacter sp. | |
| amorphus | |
| amycolatopsis | |
| amycolatopsis iriomotensis | |
| amycolatopsis spp. | |
| amycolatopsis vancoresmycina | |
| anaerolineales | |
| anaeromyxobacter | |
| anaeromyxobacter spp. | |
| anaeromyxobacteraceae | |
| ancylobacter | |
| ancylobacter spp. | |
| angustibacter peucedani | |
| aquabacterium spp. | |
| aquicella | |
| arenimonas oryziterrae | |
| armatimonadetes | |
| arsenicicoccus | |
| arsenicicoccus dermatophilus sp. | |
| arthrobacter | |
| arthrobacter pascens | |
| arthrobacter tumbae | |
| asanoa ishikariensis | |
| azohydromonas australica | |
| azonexus sp. | |
| azospira | |
| azospira oryzae | |
| azospira spp. | |
| azospirillum lipoferum | |
| azotobacter chroococcum | |
| bacillaceae | |
| bacillales | |
| bacilli | |
| bacillus | |
| bacillus acidiceler | |
| bacillus senegalensis | |
| bacillus sp. | |
| bacillus spp. | |
| bacteroidales | |
| bauldia | |
| bauldia consociata | |
| bdellovibrionaceae | |
| beijerinckia spp. | |
| blastococcus sp. | |
| blastococcus spp. | |
| blastomonas | |
| blastomonas spp. | |
| bordetella hinzii | |
| bosea sp. | |
| bradyrhizobiaceae | |
| bradyrhizobium elkanii | |
| bradyrhizobium sp. | |
| bradyrhizobium spp. | |
| bradyrhizobium yuanmingense | |
| brevundimonas | |
| brevundimonas lenta | |
| brucellaceae | |
| bryobacter | |
| burkholderia | |
| burkholderia sp. | |
| burkholderia spp. | |
| burkholderiaceae | |
| burkholderiales | |
| buttiauxella izardii | |
| byssovorax | |
| caldilinea | |
| caldilinea spp. | |
| caldilineaceae | |
| caldilineales | |
| candidatus brocadiaceae | |
| candidatus koribacter | |
| candidatus nitrosoarchaeum | |
| candidatus nitrosoarchaeum limnia | |
| candidatus phytoplasma phytoplasma sp. | |
| ryl_gd | |
| candidatus saccharibacteria | |
| candidatus solibacter | |
| candidatus solibacter uncultured solibacter | |
| sp. | |
| candidatus solibacter usitatus | |
| carnobacterium spp. | |
| catellatospora citrea | |
| catellatospora sp. | |
| catellatospora spp. | |
| catenuloplanes spp. | |
| caulobacter sp. | |
| caulobacter tundrae | |
| caulobacteraceae | |
| caulobacterales | |
| cellulomonas terrae | |
| cellvibrio vulgaris | |
| chelatococcus asaccharovorans | |
| chelatococcus spp. | |
| chitinophagaceae | |
| chloroflexaceae | |
| chloroflexales | |
| chloroflexi | |
| chloroflexia | |
| chloroflexus | |
| chloroflexus spp. | |
| chromobacteriaceae | |
| chryseobacterium | |
| chryseobacterium indologenes | |
| chthoniobacter flavus | |
| citrobacter spp. | |
| clavibacter michiganensis | |
| clostridia | |
| clostridiaceae | |
| clostridiales | |
| clostridium | |
| clostridium bowmanii | |
| clostridium gasigenes | |
| clostridium spp. | |
| clostridium vincentii | |
| comamonadaceae | |
| comamonas | |
| comamonas koreensis | |
| conexibacter | |
| conexibacter spp. | |
| conexibacter woesei | |
| conexibacteraceae | |
| coxiellaceae | |
| crenotrichaceae | |
| cryobacterium mesophilum | |
| cryobacterium sp. | |
| cryomorphaceae | |
| cupriavidus | |
| cupriavidus campinensis | |
| cyanobacteria | |
| cystobacter sp. | |
| cystobacter spp. | |
| cystobacteraceae | |
| cytophaga spp. | |
| cytophagaceae | |
| cytophagales | |
| dehalococcoidales | |
| dehalococcoides | |
| dehalococcoidia | |
| dehalogenimonas spp. | |
| denitratisoma | |
| denitratisoma spp. | |
| derxia | |
| derxia spp. | |
| desulfatiglans | |
| desulfobacteraceae | |
| desulfoglaeba spp. | |
| desulfosporosinus meridiei | |
| desulfuromonadaceae | |
| desulfuromonadales | |
| desulfuromonas | |
| devosia insulae | |
| devosia sp. | |
| devosia spp. | |
| dickeya zeae | |
| dyadobacter sp. | |
| elusimicrobia | |
| elusimicrobiaceae | |
| elusimicrobiales | |
| endomicrobia | |
| enhygromyxa salina | |
| epilithonimonas sp. | |
| erwinia persicina | |
| exiguobacterium undae | |
| ferrimicrobium | |
| ferrimicrobium spp. | |
| fictibacillus | |
| flavisolibacter spp. | |
| flavobacteriaceae | |
| flavobacteriales | |
| flavobacterium | |
| flavobacterium arsenatis | |
| flavobacterium columnare | |
| flavobacterium hauense | |
| flavobacterium johnsoniae | |
| flavobacterium sp. | |
| flavobacterium spp. | |
| flavobacterium terrigena | |
| flexibacter | |
| flexibacter spp. | |
| fodinicola spp. | |
| frankia | |
| frankia spp. | |
| frankiaceae | |
| frigoribacterium sp. | |
| gaiella | |
| gaiella occulta | |
| gaiella spp. | |
| gaiellaceae | |
| gaiellales | |
| gallionella | |
| gallionellaceae | |
| gammaproteobacteria | |
| gemmatimonadaceae | |
| gemmatimonadales | |
| gemmatimonas | |
| gemmatimonas sp. | |
| gemmatimonas spp. | |
| geobacillus sp. | |
| geobacter | |
| geobacter sp. | |
| geobacter spp. | |
| geobacteraceae | |
| halomonas muralis | |
| herbaspirillum huttiense | |
| herbaspirillum sp. | |
| herbaspirillum spp. | |
| holophaga | |
| holophaga spp. | |
| holophagaceae | |
| humibacillus xanthopallidus | |
| hydrogenophaga palleronii | |
| hydrogenophilaceae | |
| hyphomicrobiaceae | |
| hyphomicrobium | |
| hyphomicrobium sp. | |
| hyphomonas | |
| iamia | |
| iamia sp. | |
| iamia spp. | |
| iamiaceae | |
| ideonella sp. | |
| ignavibacteriaceae | |
| ignavibacteriales | |
| ignavibacterium | |
| ignavibacterium spp. | |
| ilumatobacter | |
| ilumatobacter spp. | |
| intrasporangium oryzae | |
| jiangella | |
| kaistia | |
| kallotenuales | |
| kineococcus sp. | |
| kineosporia mikuniensis | |
| kofleria | |
| kofleria spp. | |
| kofleriaceae | |
| kribbella karoonensis | |
| kribbella sp. | |
| kribbella swartbergensis | |
| labedella sp. | |
| labilithrix luteola | |
| labilitrichaceae | |
| lactobacillus | |
| lactococcus garvieae | |
| lapillicoccus jejuensis | |
| legionellaceae | |
| leifsonia kribbensis | |
| leifsonia spp. | |
| lentzea albida | |
| leptothrix sp. | |
| leptothrix spp. | |
| leucobacter tardus | |
| lysinibacillus sphaericus | |
| lysobacter sp. | |
| lysobacter spp. | |
| marinimicrobium | |
| marinobacter | |
| massilia | |
| massilia sp. | |
| massilia timonae | |
| melioribacter | |
| melioribacter spp. | |
| melioribacteraceae | |
| mesorhizobium loti | |
| mesorhizobium plurifarium | |
| mesorhizobium sp. | |
| mesorhizobium spp. | |
| methylibium | |
| methylobacillus flagellatus | |
| methylobacillus spp. | |
| methylobacter spp. | |
| methylobacteriaceae | |
| methylobacterium adhaesivum | |
| methylobacterium spp. | |
| methylocella spp. | |
| methylococcaceae | |
| methylococcales | |
| methyloversatilis | |
| methyloversatilis spp. | |
| microbacteriaceae | |
| microbacterium kitamiense | |
| microbacterium sp. | AP017975.1 |
| microcella alkaliphila | |
| micrococcaceae | |
| microlunatus spp. | |
| micromonospora rhodorangea | |
| micromonosporaceae | |
| microvirga aerilata | |
| microvirga subterranea | |
| moorella spp. | |
| mycobacterium sacrum | |
| mycobacterium salmoniphilum | |
| mycobacterium septicum | |
| mycobacterium spp. | |
| nakamurella sp. | |
| nannocystaceae | |
| nannocystis | |
| nannocystis exedens | |
| neorhizobium rhizobium huautlense | |
| niastella spp. | |
| nitrosomonadaceae | |
| nitrosomonas spp. | |
| nitrosomonas ureae | |
| nitrosopumilaceae | |
| nitrosospira | |
| nitrosospira spp. | |
| nitrosovibrio tenuis | |
| nitrospira enrichment | |
| nitrospira sp. | |
| nitrospira spp. | |
| nitrospiraceae | |
| nitrospirales | |
| nocardia anaemiae | |
| nocardia pneumoniae | |
| nocardioidaceae | |
| nocardioides iriomotensis | |
| nocardioides islandensis | |
| nocardioides maritimus | |
| nocardioides perillae | |
| nocardioides sp. | |
| nocardioides spp. | |
| nordella | |
| nordella spp. | |
| novosphingobium sp. | |
| novosphingobium spp. | |
| ochrobactrum haematophilum | |
| ohtaekwangia spp. | |
| olivibacter soli | |
| opitutaceae | |
| oryzihumus spp. | |
| oxalobacteraceae | |
| paenibacillaceae | |
| paenibacillus | |
| paenibacillus sp. | |
| pantoea agglomerans | CP016889.1 |
| paracoccus spp. | |
| paracraurococcus sp. | |
| parastreptomyces | |
| pasteuriaceae | |
| pedobacter kribbensis | |
| pedobacter kwangyangensis | |
| pedobacter sp. | |
| pedobacter spp. | |
| pedobacter tournemirensis | |
| pedosphaera | |
| pedosphaera spp. | |
| pelobacter | |
| pelobacter spp. | |
| peredibacter spp. | |
| phaselicystidaceae | |
| phenylobacterium | |
| phenylobacterium sp. | |
| phenylobacterium spp. | |
| phycicoccus sp. | |
| phycisphaerae | |
| phycisphaerales | |
| phyllobacterium | |
| phyllobacterium spp. | |
| phyllobacterium trifolii | |
| pigmentiphaga sp. | |
| pirellula spp. | |
| planctomycetaceae | |
| planctomycetales | |
| planctomycetia | |
| planococcus spp. | |
| plesiocystis spp. | |
| polaromonas spp. | |
| procabacteriales | |
| promicromonospora sp. | |
| promicromonospora sukumoe | |
| prosthecobacter spp. | |
| prosthecomicrobium spp. | |
| pseudoalteromonas | |
| pseudoclavibacter helvolus | |
| pseudolabrys | |
| pseudolabrys spp. | |
| pseudolabrys taiwanensis | |
| pseudomonadaceae | |
| pseudomonadales | |
| pseudomonas | |
| pseudomonas flavescens | |
| pseudomonas fluorescens | |
| pseudonocardia | |
| pseudonocardia carboxydivorans | |
| pseudonocardia sp. | |
| pseudonocardia spp. | |
| pseudonocardia zijingensis | |
| pseudorhodoferax sp. | |
| pseudoxanthobacter | |
| pseudoxanthomonas spp. | |
| ralstonia spp. | |
| ramlibacter sp. | |
| ramlibacter spp. | |
| reyranella massiliensis | |
| reyranella sp. | |
| rheinheimera sp. | |
| rhizobiaceae | |
| rhizobiales | |
| rhizobium | |
| rhizobium etli | |
| rhizobium sp. | |
| rhizobium spp. | |
| rhizomicrobium spp. | |
| rhodobacter spp. | |
| rhodobiaceae | |
| rhodococcus kroppenstedtii | |
| rhodococcus spp. | |
| rhodococcus wratislaviensis | |
| rhodocyclaceae | |
| rhodocyclales | |
| rhodomicrobium | |
| rhodomicrobium spp. | |
| rhodoplanes | |
| rhodoplanes sp. | |
| rhodoplanes spp. | |
| rhodopseudomonas spp. | |
| rhodospirillaceae | |
| rhodospirillales | |
| rhodothermus | |
| rickettsiaceae | |
| roseateles | |
| roseateles spp. | |
| rubrivivax | |
| rubrivivax gelatinosus | |
| rubrivivax spp. | |
| rubrobacter | |
| ruminococcaceae | |
| saccharopolyspora | |
| saccharopolyspora gloriosa | |
| saccharopolyspora sp. | |
| sandaracinus | |
| saprospiraceae | |
| serratia proteamaculans | CP000826.1 |
| shimazuella | |
| shinella granuli | |
| sideroxydans lithotrophicus | |
| sideroxydans paludicola | |
| sinobacteraceae | |
| sinorhizobium sp. | |
| solibacteraceae | |
| solirubrobacter | |
| solirubrobacter spp. | |
| sorangium | |
| sorangium cellulosum | |
| sphaerobacteraceae | |
| sphaerobacterales | |
| sphingobacteriaceae | |
| sphingobacteriales | |
| sphingobacterium | |
| sphingobium herbicidovorans | |
| sphingobium xenophagum | |
| sphingomonadaceae | |
| sphingomonadales | |
| sphingomonas | |
| sphingomonas spp. | |
| sphingomonas wittichii | |
| sphingopyxis macrogoltabida | |
| sphingosinicella | |
| sporichthya | |
| sporichthya spp. | |
| sporichthyaceae | |
| stackebrandtia nassauensis | |
| stenotrophomonas maltophilia | |
| steroidobacter | |
| steroidobacter spp. | |
| stigmatella erecta | |
| streptomyces | |
| streptomyces aculeolatus | |
| streptomyces fradiae | |
| streptomyces ghanaensis | |
| streptomyces hebeiensis | |
| streptomyces mashuensis | |
| streptomyces microflavus | |
| streptomyces netropsis | |
| streptomyces sp. | |
| streptomyces spp. | |
| streptomyces variabilis | |
| streptomyces vayuensis | |
| streptomyces viridochromogenes | |
| streptomyces viridodiastaticus | |
| streptomyces xinghaiensis | |
| streptomyces xylophagus | |
| streptomycetaceae | |
| sulfuricella | |
| syntrophaceae | |
| syntrophobacter wolinii | |
| syntrophorhabdaceae | |
| syntrophorhabdus | |
| syntrophus spp. | |
| taibaiella sp. | |
| tepidamorphus | |
| tepidamorphus spp. | |
| terrabacter | |
| terrabacter sp. | |
| terrabacter spp. | |
| terriglobus | |
| terrimonas sp. | |
| terrimonas spp. | |
| tetrasphaera | |
| tetrasphaera elongata | |
| thermomonosporaceae | |
| thiobacillus | |
| thiobacillus denitrificans | |
| thiobacillus spp. | |
| thiobacter spp. | |
| thiomonas | |
| thiorhodovibrio spp. | |
| uncultured candidatus koribacter sp. | |
| variovorax paradoxus | |
| verrucomicrobia subdivision 3 | |
| verrucomicrobiaceae | |
| verrucomicrobiales | |
| woodsholea | |
| woodsholea maritima | |
| xanthomonadaceae | |
| xanthomonadales | |
| xanthomonas | |
| xanthomonas spp. | |
| zoogloea | |
| zooshikella | |

Table 4 shows bacterial strains that can be useful in the microbial inoculant compositions and methods disclosed herein.

TABLE 4
Microbes.

| Species | GenBank Accession No. |
|---|---|
| Agrobacterium tumefaciens | AE007869.2 |
| Arthrobacter sp. | CP022436.1 |
| Agrobacterium rhizogenes | CP019701.1 |
| Bacillus megaterium | CP001983.1 |
| Bacillus megaterium | CP018874.1 |
| Erwinia sp. | CP002124.1 |
| Microbacterium testaceum | AP012052.1 |
| Microbacterium sp. | AP017975.1 |
| Pseudomonas sp. | LT707063.1 |
| Pantoea agglomerans | CP016889.1 |
| Pseudomonas sp. | LT707063.1 |
| Pseudomonas mandelii | CP005960.1 |
| Sphingomonas sp. | CP023705.1 |
| Sphingomonas melonis | CP023705.1 |
| Serratia proteamaculans | CP000826.1 |

In some aspects, a microbial inoculant compositions can further comprise one or more of yeast strain TAH3020 or yeast strain TAH3021.

Disclosed herein are microbial inoculant compositions that include additional microbial species or other additives to induce the plant to perform desired physiological, metabolic, or other activity. For example, in some aspects, the microbial inoculant compositions can include one or more of the following microbial species: an Acetobacteraceae, spp. (e.g., Acidisphaera spp.), an Acetivibrio spp. (e.g., Acetivibrio cellulolyticus), an Acidiphilium spp., an Acidimicrobiaceae spp. (e.g., an Acidimicrobium spp., an Aciditerrimonas spp.), an Acidobacteriales spp. (e.g., an Acidobacteriaceae spp. [e.g., an Acidobacterium spp.]), an Acidothermus spp., an Acidovorax spp. (e.g., Acidovorax citrulli), an Acinetobacter spp. (e.g., Acinetobacter lwoffii), an Actinoallomurus spp. (e.g., Actinoallomurus iriomotensis), an Actinocatenispora spp. (e.g., Actinocatenispora rupis), an Actinomadura spp., an Actinomycetales spp. (e.g., an Actinomyces spp.), an Actinoplanes spp. (e.g., Actinoplanes auranticolor), an Actinopolymorpha spp. (e.g., Actinopolymorpha pittospori), an Actinotalea spp. (e.g., Actinotalea fermentans), an Adhaeribacter spp. (e.g., Adhaeribacter terreus), an Aeromicrobium spp. (e.g., Aeromicrobium fastidiosum), an Afipia spp., an Agromyces spp. (e.g., Agromyces ulmi, Agromyces subbeticus), an Alcaligenaceae spp., an Algoriphagus spp., an Alkaliflexus spp., an Alphaproteobacteria spp., an Alsobacter spp. (e.g., Alsobacter metallidurans), an Altererythrobacter spp., an Alteromonadaceae spp., an Amaricoccus spp., an Aminobacter spp., an Amycolatopsis spp. (e.g., Amycolatopsis iriomotensis, Amycolatopsis vancoresmycina), an Anaeromyxobacteraceae spp. (e.g., an Anaeromyxobacter spp. [e.g., Anaeromyxobacter dehalogenans]), an Ancylobacter spp., an Angustibacter spp. (e.g., Angustibacter peucedani), an Aquabacterium spp., an Aquicella spp., an Armatimonadetes spp., an Arenimonas spp. (e.g., Arenimonas oryziterrae), an Arsenicicoccus spp. (e.g., Arsenicicoccus dermatophilus), an Arthrobacter spp. (e.g., Arthrobacter pascens, Arthrobacter tumbae), an Asanoa spp. (e.g., Asanoa ishikariensis), an Azohydromonas spp. (e.g., Azohydromonas australica), an Azonexus spp., an Azospira spp. (e.g., Azospira oryzae), an Azospirillum spp. (e.g., Azospirillum lipoferum), an Azotobacter spp. (e.g., Azotobacter chroococcum), a Bacillaceae spp. (e.g., a Bacillus spp. [e.g., Bacillus acidiceler, Bacillus aphidicola, Bacillus senegalensis, Bacillus megaterium, Bacillus subtilis]), a Bacteroidetes spp. (e.g., a Bacteroidales spp. [e.g., a Bacteroides spp.]), a Bauldia spp. (e.g., Bauldia consociate), a Bdellovibrionaceae spp., a Beijerinckia spp., a Blastococcus spp. (e.g., Blastococcus saxobsidens), a Blastomonas spp., a Bordetella spp. (e.g., Bordetella hinzii), a Bosea spp., a Bradyrhizobiaceae, spp. (e.g., Bradyrhizobium spp. [e.g., Bradyrhizobium elkanii, Bradyrhizobium yuanmingense]), a Brevibacteriaceae spp., a Brevundimonas spp. (e.g., Brevundimonas lenta), a Bryobacter spp., a Burkholderiales spp. (e.g., a Burkholderiaceae spp. [e.g., a Burkholderia spp.]), a Brucellaceae spp., a Buttiauxella spp. (e.g., Buttiauxella izardii), a Byssovorax, spp., a Caldilineales spp. (e.g., a Caldilineaceae spp. [e.g., a Caldilinea spp.]), a Caloramator spp., a Candidatus spp. (e.g., Candidatus brocadiaceae, Candidatus entotheonella, koribacter, Candidatus Candidatus phytoplasma, Candidatus saccharibacteria, Candidatus nitrosoarchaeum, Candidatus solibacter), a Carnobacterium spp., a Catenuloplanes spp., a Catellatospora spp., (e.g., Catellatospora citrea), a Caulobacteraceae spp. (e.g., a Caulobacter spp. [e.g., Caulobacter tundrae]), a Cellulosimicrobium spp. (e.g., Cellulosimicrobium cellulans), a Cellvibrio spp. (e.g., Cellvibrio vulgaris), a Cellulomonas spp. (e.g., Cellulomonas terrae), a Chelatococcus spp. (e.g., Chelatococcus asaccharovorans, a Chitinophagaceae spp., a Chromobacteriaceae spp., a Chloroflexales spp. (e.g., a Chloroflexaceae spp. [e.g., a Chloroflexus spp.]), a Chthoniobacter spp. (e.g., Chthoniobacter flavus), a Chryseobacterium spp., a Citrobacter spp., a Clavibacter spp. (e.g., Clavibacter michiganensis), a Clostridiaceae spp. (e.g., a Clostridium spp. [e.g., Clostridium bowmanii, Clostridium gasigenes, Clostridium uliginosum, Clostridium vincentii]), a Comamonadaceae spp. (e.g., a Comamonas, spp. [e.g., Comamonas koreensis]), a Conexibacteraceae spp. (e.g., a Conexibacter spp. [e.g., Conexibacter woesei]), a Coxiellaceae spp., a Crenotrichaceae spp. a Cryomorphaceae spp., a Cryobacterium spp. (e.g., Cryobacterium mesophilum), a Cupriavidus spp. (e.g., Cupriavidus campinensis), a Curtobacterium spp., a Cyanobacteria spp., a Cyclobacteriaceae spp., a Cystobacteraceae spp. (e.g., a Cystobacter spp.), a Cytophagaceae spp. (e.g., a Cytophaga spp.), a Defluviicoccus spp., a Dehalococcoidales spp. (e.g., a Dehalogenimonas spp., a Dehalococcoides spp.), a Denitratisoma spp., a Derxia spp., a Desulfovibrionales spp. (e.g., a Desulfobacteraceae spp. [e.g., a Desulfocapsa spp., a Desulfatiglans spp., a Desulforegula spp.]), a Desulfoglaeba spp., a Desulfosporosinus spp. (e.g., Desulfosporosinus meridiei), a Desulfotomaculum spp., a Desulfuromonadales spp. (e.g., a Desulfuromonas spp.), a Devosia spp. (e.g., Devosia insulae), a Dickeya spp. (e.g., Dickeya zeae), a Dyadobacter spp., an Ectothiorhodospiraceae spp., an Elusimicrobia spp. (e.g., an Elusimicrobiaceae spp. [e.g., an Elusimicrobium spp.]), an Endomicrobia spp., an Enhygromyxa spp. (e.g., Enhygromyxa salina), an Epilithonimonas spp., an Erwinia spp. (e.g., Erwinia persicina), an Exiguobacterium spp. (e.g., Exiguobacterium undae), a Ferrimicrobium spp., a Fictibacillus spp., a Flavobacteriales spp. (e.g., a Flavobacteriaceae, [e.g., a Flavobacterium spp. such as, for example, Flavobacterium arsenatis, Flavobacterium columnare, Flavobacterium hauense, Flavobacterium johnsoniae, Flavobacterium terrigena]), a Flavisolibacter spp., a Flexibacter spp., a Flindersiella spp., a Fodinicola spp., a Frankia spp., Frigoribacterium spp., a Gaiellales spp. (e.g., a Gaiella spp. [e.g., Gaiella occulta]), a Gallionellaceae spp. (e.g., a Gallionella spp.), a Gemmatimonadales spp. (e.g., a Gemmatimonadaceae spp. [a Gemmatimonas spp.]), a Gemmata spp., a Geoalkalibacter spp., a Geobacillus spp., a Geobacteraceae spp. (e.g., a Geobacter spp.), a Gillisia spp., a Glycomyces spp. (e.g., Glycomyces harbinensis), a Halomonas spp. (e.g., Halomonas muralis), a Haliangium spp., a Herbaspirillum spp. (e.g., Herbaspirillum huttiense), a Holophagales spp. (e.g., a Holophagaceae spp. [e.g., a Holophaga spp.]), a Humibacillus spp. (e.g., Humibacillus xanthopallidus), a Hydrogenophaga spp. (e.g., Hydrogenophaga palleronii), a Hydrogenophilaceae spp., a Hyphomicrobiaceae spp. (e.g., a Hyphomicrobium spp. [e.g., Hyphomicrobium methylovorum]), a Hyphomonas spp., an lamiaceae spp. (e.g., an lamia spp.), an Ideonella spp., an Ignavibacteriales spp. (e.g., an Ignavibacteriaceae spp. such as, for example, an Ignavibacterium spp.), an Ilumatobacter spp., an Intrasporangiaceae spp. (e.g., an Intrasporangium spp. [e.g., Intrasporangium oryzae]), a Jiangella spp., a Kaistia spp., a Kaistobacter spp., a Kallotenuales spp., a Kineococcus spp., a Kineosporia spp. (e.g., Kineosporia mikuniensis), a Knoellia spp., a Kofleriaceae spp. (e.g., a Kofleria spp.), a Kribbella spp. (e.g., Kribbella karoonensis, Kribbella swartbergensis), a Labedella spp., a Labilitrichaceae spp. (e.g., a Labilithrix spp. [e.g., Labilithrix luteola]), a Lactobacillus spp., a Lactococcus spp. (e.g., Lactococcus garvieae), a Lapillicoccus spp. (e.g., Lapillicoccus jejuensis), a Legionellaceae spp., a Leifsonia spp., a Lentzea spp. (e.g., Lentzea albida), a Leptospira spp., a Leptothrix spp., a Leucobacter spp. (e.g., Leucobacter tardus), a Longilinea spp., a Lysinibacillus spp. (e.g., Lysinibacillus sphaericus), a Lysobacter spp., a Marinimicrobium spp., a Marinobacter spp., a Marmoricola spp., a Massilia spp. (e.g., Massilia timonae), a Melioribacteraceae spp. (e.g., a Melioribacter spp.), a Mesorhizobium spp. (e.g., Mesorhizobium loti, Mesorhizobium plurifarium), a Methylibium spp., a Methylobacillus spp. (e.g., Methylobacillus flagellates), a Methylobacteriaceae spp. (e.g., a Methylobacterium spp. [e.g., Methylobacterium adhaesivum]), a Methylocella spp., a Methylococcaceae spp. (e.g., a Methylobacter spp.), a Methylocystaceae spp. (e.g., a Methylocystis spp. [e.g., Methylocystis echinoides]), a Methylosinus spp., a Methyloversatilis spp., a Microbacteriaceae spp. (e.g., a Microbacterium spp. [e.g., Microbacterium kitamiense], a Microcella spp. [e.g., Microcella alkaliphile]), a Micrococcaceae spp., a Microlunatus spp., a Microvirga spp. (e.g., Microvirga aerilata, Microvirga subterranean), a Mycobacteriaceae spp. (e.g., a Mycobacterium spp. [e.g., Mycobacterium sacrum, Mycobacterium salmoniphilum, Mycobacterium septicum]), a Micromonosporaceae spp. (e.g., a Micromonospora spp. [e.g., Micromonospora rhodorangea]), a Modestobacter spp. (e.g., Modestobacter multiseptatus), a Moorella spp., a Myxococcales spp., a Nakamurella spp., a Nannocystaceae spp. (e.g., a Nannocystis spp. [e.g., Nannocystis exedens]), a Neorhizobium spp. (e.g., Neorhizobium huautlense), a Niastella spp., a Nitriliruptor spp., a Nitrosomonadaceae spp. (e.g., a Nitrosomonas spp. [e.g., Nitrosomonas communis, Nitrosomonas ureae]), a Nitrosopumilales spp. (e.g., a Nitrosopumilaceae spp.), a Nitrosospira spp., a Nitrosovibrio spp. (e.g., Nitrosovibrio tenuis), a Nitrospirales spp. (e.g., a Nitrospira spp.), a Nocardiaceae spp. (e.g., a Nocardia spp. [e.g., Nocardia anaemiae]), a Nocardioidaceae spp. (e.g., a Nocardioides spp. [e.g., Nocardioides albus, Nocardioides iriomotensis, Nocardioides islandensis, Nocardioides maritimus, Nocardioides perillae, Nocardia pneumoniae]), a Nocardiopsis spp. (e.g., Nocardiopsis synnemalaformans), a Nonomuraea spp. (e.g., Nonomuraea kuesteri), a Nordella spp., a Novosphingobium spp., an Ochrobactrum spp. (e.g., Ochrobactrum haematophilum), an Ohtaekwangia spp., an Olivibacter spp. (e.g., Olivibacter soli), an Opitutaceae spp., an Oryzihumus spp., an Oxalobacteraceae spp., an Oxalophagus spp. (e.g., Oxalophagus oxalicus), a Paenibacillus spp., (e.g., Paenibacillus graminis, Paenibacillus chondroitinus, Paenibacillus validus), a Pantoea spp. (e.g., Pantoea agglomerans), a Paracoccus spp., a Paracraurococcus spp., a Parastreptomyces spp., a Pasteuriaceae spp., (e.g., a Pasteuria spp.), a Pedosphaera spp. (e.g., Pedosphaera parvula), a Pedobacter spp. (e.g., Pedobacter tournemirensis, Pedobacter kribbensis, Pedobacter kwangyangensis), a Pelagibacterium spp. (e.g., Pelagibacterium halotolerans), a Pelobacteraceae spp. (e.g., a Pelobacter spp.), a Peptoclostridium spp. (e.g., Peptoclostridium clostridium sordellii), a Peredibacter spp., a Phaselicystidaceae spp., a Phenylobacterium spp., a Phycicoccus spp., a Phycisphaerae spp., a Phyllobacterium spp. (e.g., Phyllobacterium trifolii), a Pigmentiphaga spp., a Planococcus spp., a Planomicrobium spp., (e.g., Planomicrobium novatatis), a Planctomycetes spp. (e.g., a Pirellula spp., such as Pirella staleyi), a Plesiocystis spp., a Polaromonas spp., a Polyangiaceae spp., a Procabacteriacae Prolixibacter spp., a Promicromonospora spp., (e.g., Promicromonospora sukumoe), a Prosthecobacter spp., a Prosthecomicrobium spp., a Pseudoalteromonas spp., a Pseudoclavibacter spp., (Pseudoclavibacter helvolus), a Pseudolabrys spp., (e.g., Pseudolabrys taiwanensis), a Pseudomonadaceae spp. (e.g., Pseudomonas fluorescens, Pseudomonas flavescens, Pseudomonas protegens, Pseudomonas veronii, Pseudomonas rhodesiae, Pseudomonas koreensis, Pseudomonas moorei, Pseudomonas baetica), a Pseudonocardia spp., (e.g., Pseudonocardia zijingensis, Pseudonocardia carboxydivorans), a Pseudorhodoferax spp., a Pseudoxanthobacter spp., a Pseudoxanthomonas spp., a Ralstonia spp., a Ramlibacter spp., a Reyranella spp. (e.g., Reyranella massiliensis), a Rheinheimera spp., a Rhizobiales spp. (e.g., a Rhizobiaceae spp., a Rhodobiaceae spp.), a Rhizobium spp. (e.g., Rhizobium etli), a Rhizomicrobium spp., a Rhodobacterales spp. (e.g., a Rhodobacter spp.), a Rhodococcus spp. (e.g., Rhodococcus gordoniae, Rhodococcus kroppenstedtii, Rhodococcus wratislaviensis), a Rhodocyclales spp. (e.g., a Rhodocyclaceae spp.), a Rhodomicrobium spp., a Rhodoplanes spp. (e.g., Rhodoplanes elegans), a Rhodopseudomonas spp., a Rhodospirillales spp. (e.g., a Rhodospirillaceae spp.), a Rhodothermus spp., a Rickettsiaceae spp., a Roseateles spp., a Roseomonas spp., a Rubrivivax spp. (e.g., Rubrivivax gelatinosus), a Rubrobacterales spp. (e.g., a Rubrobacter spp.), a Ruminococcaceae spp., a Saccharopolyspora spp. (e.g., Saccharopolyspora gloriosa), a Sandaracinus spp., a Saprospiraceae spp., a Serratia spp. (e.g., Serratia proteamaculans), a Shimazuella spp. (e.g., Shimazuella kribbensis), a Shinella spp. (e.g., Shinella granuli), a Sideroxydans spp. (e.g., Sideroxydans lithotrophicus, Sideroxydans paludicola), a Sinobacteraceae spp. (e.g., a Steroidobacter spp.), a Sinorhizobium spp., a Solibacteraceae spp. (e.g., a Solibacter spp.), a Solirubrobacteraceae spp. (e.g., a Solirubrobacter spp.), a Sorangium spp. (e.g., Sorangium cellulosum), a Sphaerobacterales spp. (e.g., a Sphaerobacteraceae spp. such as, for example, a Sphaerobacter spp.), a Sphingobacteriales spp. (e.g., a Sphingobacteriaceae spp. such as, for example, a Sphingobacterium spp.), a Sphingobium spp. (e.g., Sphingobium herbicidovorans), a Sphingomonadaceae spp. (e.g., a Sphingobium spp. [e.g., S. xenophagum], a Sphingomonas spp. [e.g., S. wittichii]), a Sphingopyxis spp. (e.g., Sphingopyxis macrogoltabida), a Sphingosinicella spp., a Spirochaetales spp. (e.g., a Spirochaeta spp.), a Sporichthyaceae spp. (e.g., a Sporichthya spp.), a Stackebrandtia spp. (e.g., Stackebrandtia nassauensis, a Stella spp., a Stenotrophomonas spp. (e.g., Stenotrophomonas maltophilia), a Stigmatella spp. (e.g., Stigmatella erecta), a Streptacidiphilus spp., a Streptoalloteichus spp., a Streptomycetaceae spp. (e.g., a Streptomyces spp. [e.g., Streptomyces aculeolatus, Streptomyces clavuligerus, Streptomyces fradiae, Streptomyces ghanaensis, Streptomyces glauciniger, Streptomyces hebeiensis, Streptomyces heteromorphus, Streptomyces mashuensis, Streptomyces microflavus, Streptomyces netropsis, Streptomyces phaeochromogenes, Streptomyces roseogriseolus, Streptomyces variabilis, Streptomyces vayuensis, Streptomyces viridodiastaticus, Streptomyces viridochromogenes, Streptomyces xylophagus, Streptomyces xinghaiensis]), a Sulfuricella spp., a Syntrophobacterales spp. (e.g., a Syntrophorhabdaceae spp. such as, for example, a Syntrophobacter spp. [e.g., S. wolinii], a Syntrophorhabdus spp., a Syntrophaceae spp., a Syntrophus spp.), a Taibaiella spp., a Tepidamorphus spp., a Terrabacter spp., a Terriglobus spp., a Terrimonas spp., a Tetrasphaera spp. (e.g., Tetrasphaera elongate), a Thermoanaerobacterales spp. (e.g., a Thermoanaerobacteraceae spp.), a Thermoflavimicrobium spp., a Thermoleophilaceae spp., a Thermomonosporaceae spp., a Thioalkalivibrio spp., a Thiobacillus spp., (e.g., Thiobacillus denitrificans), a Thiobacter spp., a Thiomonas spp., a Thiorhodovibrio spp., a Tolumonas spp., (e.g., Tolumonas auensis) a Variovorax spp., (e.g., Variovorax paradoxus), a Verrucomicrobiales spp., (e.g., a Verrucomicrobia subdivision 3 spp.), a Vibrionales spp., a Woodsholea spp., (e.g., Woodsholea maritima), a Xanthomonadaceae spp., (e.g., a Xanthomonas spp.), a Zoogloea spp., or a Zooshikella spp.

In some aspects, the following can act as an antagonist to at least one of the microbial species listed above, e.g., such as Pseudomonas fluorescens, Pseudomonas Streptornyces hygroscopicus, Mycobacterium vaccae, Agrobacterium turnefaciens, Bacillus megaterium, Bacillus amyloliquifaciens, Bacillus subtilus, Bacillus pumilus, a Shingomonas spp., Sphingomonas melonis, an Arthrobacter spp., Agrobacterium rhizogenes, Serratia proteatnaculans Microbacterium testaceum, a Pseudomonas spp., an Erwinia spp., Pantoea agglomerans, Pseudomonas inandelii, a Microbacterium spp., Clostridium saccharobutylicum, Pseudomonas moraviensis, Pantoea vagans, Serratia liquefaciens, Pedobacter kribbensis, Tolumonas auensis, Janthinobacterium lividum, Bacillus racemilacticus, Sporolactobacillus laevolacticus, Brevundimonas mediterranea, Pantoea cloacae, Clostridium acidisoli, Erwinia aphidicola, Bacillus arbutinivorans, Paenibacillus grarninis Pseudomonas veronii, Pseudomonas rhodesiae, Pseudomonas koreensis, Tolumonas auensis, Pseudomonas moorei, Pseudomonas baetica, and/or Pseudomonas protegens.

In some aspects, a microbial species that provides insecticidal activity can be added to the microbial inoculant. Suitable microbes can include bacteria or fungi that produce phytochemicals that have insecticidal or insect repelling properties. In some aspects, the microbial species can be a bacterium such as, for example, B. thuringiensis, B. pipilliae, Photohabdus luminescens, Pseudomonas entomohpilia, Envinia aphidicola, etc., or a fungus such as, for example, Beaveria bassiana, Lagenidium giganteum, etc.

Disclosed herein are microbial inoculant compositions comprising one or more non-microbial additives. For example, a microbial inoculant composition can include one or more macronutrients or one or more micronutrients such as, for example, carbon, nitrogen, potassium, phosphorus, zinc, magnesium, selenium, chromium, tin, manganese, cobalt, zinc, and/or copper.

The microbes may be incubated at a minimum temperature of at least 5° C., such as, for example, at least 10° C., at least 15° C. at least 20° C., at least 25° C., at least 30° C., or at least 40° C. The microbes may be incubated at a maximum temperature of no more than 50° C., such as, for example, no more than 45° C., no more than 45° C., no more than 40° C., no more than 35° C., or no more than 30° C. The microbes may be incubated at a temperature characterized by any range that includes, as endpoints, any combination of a minimum temperature identified above and any maximum temperature identified above that is greater than the minimum temperature. For example, in some aspects, the microbes may be incubated at a temperature of from 10° C. to 40° C.

In some aspects, the microbial inoculant compositions can be prepared by incubating the microbes in a suitable culture medium for a sufficient time to allow growth of both aerobic and anaerobic microbes in the fermentation culture. When a mixture of aerobic microbes and anaerobic microbes are co-fermented, the microbes may be incubated for a minimum of at least 48 hours, such as, for example, at least 72 hours, at least 96 hours, at least 120 hours, at least 144 hours, or at least 168 hours. The microbes may be incubated for a maximum of no more than 240 hours, no more than 216 hours, no more than 192 hours, no more than 168 hours, no more than 144 hours, no more than 120 hours, or no more than 96 hours. The microbes may be incubated for a period characterized by a range having, as endpoints, any combination of a minimum incubation time listed herein and any maximum incubation time listed herein that is greater than the minimum incubation time.

### Methods for Selectively and Separately Producing Hydrogen and Methane I

Disclosed herein are methods for selectively and separately producing hydrogen and methane. Also disclosed herein are methods for selectively and separately producing hydrogen or methane. Further disclosed herein are methods for producing hydrogen.

Disclosed herein are methods for selectively and separately producing hydrogen and methane, the methods comprising: a) contacting a biomass in a first reactor vessel with a first microbial inoculant composition under anaerobic conditions to facilitate the digestion of the biomass to produce a digested biomass, wherein the first reactor vessel is maintained at a first oxidation reduction potential (ORP); b) collecting hydrogen gas from the first reactor vessel; c) transferring a portion of digested biomass from step a) to a second reactor vessel; d) introducing an oxygen-containing gas to the second reactor vessel to change the first ORP from the first reactor vessel to a second ORP in the second reactor vessel and contacting the digested biomass in the second reactor vessel with a second microbial inoculant composition under anaerobic conditions to facilitate the digestion of the digested biomass; and e) collecting biogas from the second reactor vessel, wherein the first microbial inoculant comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2.

Disclosed herein are methods for producing hydrogen and methane, the methods comprising: a) contacting a biomass in a first reactor vessel with a first microbial inoculant composition under anaerobic conditions to facilitate the digestion of the biomass to produce a digested biomass, wherein the first reactor vessel is maintained at a first oxidation reduction potential (ORP); and b) collecting hydrogen gas from the first reactor vessel, wherein the first microbial inoculant comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2.

Disclosed herein are methods for selectively and separately producing hydrogen or methane, the methods comprising: a) contacting a biomass in a first reactor vessel with a first microbial inoculant composition under anaerobic conditions to facilitate the digestion of the biomass to produce a digested biomass, wherein the first reactor vessel is maintained at a first oxidation reduction potential (ORP); b) optionally collecting hydrogen gas from the first reactor vessel; c) transferring a portion of digested biomass from step a) to a second reactor vessel; d) introducing an oxygen-containing gas to the second reactor vessel to change the first ORP from the first reactor vessel to a second ORP in the second reactor vessel and contacting the digested biomass in the second reactor vessel with a second microbial inoculant composition under anaerobic conditions to facilitate the digestion of the digested biomass; and e) collecting biogas from the second reactor vessel, wherein the first microbial inoculant comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2.

In some aspects, the methods comprise: a) contacting a biomass in a first reactor vessel with a first microbial inoculant composition under anaerobic conditions to facilitate the digestion of the biomass to produce a digested biomass. In some aspects, the first reactor vessel can be maintained at a first oxidation reduction potential (ORP). In some aspects, the methods comprise producing a partially digested biomass. In some aspects, the methods can comprise b) collecting hydrogen gas from the first reactor vessel. In some aspects, the methods can comprise c) transferring a portion of the digested biomass or the partially digested biomass from step a) to a second reactor vessel. In some aspects, the methods can comprise d) introducing an oxygen-containing gas to the second reactor vessel to change the first ORP from the first reactor vessel to a second ORP in the second reactor vessel and contacting the digested biomass or partially digested biomass in the second reactor vessel with a second microbial inoculant composition under anaerobic conditions to facilitate the digestion of the digested biomass or partially digested biomass. In some aspects, the methods can comprise e) collecting biogas from the second reactive vessel. In some aspects, the methods can further comprise f) transferring a portion of the digested biomass from step c) to a third reactor vessel and contacting the digested biomass in the third reactor vessel with a third microbial inoculant composition to facilitate the digestion of the biomass under conditions to convert ammonia into nitrates.

In some aspects, the first microbial inoculant can comprise a first bacterial strain and a second bacterial strain, wherein the first bacterial strain comprises Clostridium spp. In some aspects, the 16S sequence of Clostridium spp. can comprise any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2.

In some aspects, the first microbial inoculant composition in step a) can decrease or suppress one or more methanogens in the first reactor vessel. In some aspects, the first microbial inoculant composition can create or change the environment in the first reactor vessel such than the metabolism of any of methanogens present in the first reactor vessel is suppressed. In some aspects, the second microbial inoculant composition can create an environment in the second reactor vessel such that the metabolism of the methanogens present in the second reactor vessel is not suppressed. In some aspects, the second microbial inoculant composition present in the second reactor vessel are capable of producing methane.

In some aspects, the second microbial inoculant composition can be different than the first microbial inoculant composition. In some aspects, the second microbial inoculant composition can comprise one or more methanogen producers. Examples of methanogoen producers include but are not limited to Methanobacterium bryantii, Methanobacterium formicum, Methanobrevibacter arboriphilicus, Methanobrevibacter gottschalkii, Methanobrevibacter ruminantium, Methanobrevibacter smithii, Methanococcus chunghsingensis, Methanococcus burtonii, Methanococcus aeolicus, Methanococcus deltae, Methanococcus jannaschii, Methanococcus maripaludis, Methanococcus vannielii, Methanocorpusculum labreanum, Methanoculleus bourgensis (Methanogenium olentangyi and Methanogenium bourgense), Methanoculleus marisnigri, Methanoflorens stordalenmirensis, Methanofollis liminatans, Methanogenium cariaci, Methanogenium frigidum, Methanogenium organophilum, Methanogenium wolfei, Methanomicrobium mobile, Methanopyrus kandleri, Methanoregula boonei, Methanosaeta concilii, Methanosaeta thermophile, Methanosarcina acetivorans, Methanosarcina barkeri, Methanosarcina mazei, Methanosphaera stadtmanae, Methanospirillium hungatei, Methanothermobacter defluvii (Methanobacterium defluvii), Methanothermobacter thermautotrophicus (Methanobacterium thermoautotrophicum), Methanothermobacter thermoflexus (Methanobacterium thermoflexum), Methanothermobacter wolfei (Methanobacterium wolfei), and Methanothrix sochngenii.

In some aspects, the third microbial inoculant composition can comprise a first bacterial strain and a second bacterial strain. In some aspects, the first bacterial strain can comprise Clostridium spp. In some aspects, the 16S sequence of Clostridium spp. can comprise any one of the Clostridium spp. listed in Table 1 or Table 2. In some aspects, the second bacterial strain can comprise an aquatic Pseudomonas spp. bacteria with a 16S nucleic acid sequence that is at least about 97% identical to any one of the Pseudomonas spp. bacteria listed in Table 1 or Table 2. In some aspects, the third microbial inoculant composition can be the same as the first microbial inoculant composition in step a). In some aspects, the third microbial inoculant composition can be the same as the first microbial inoculant composition in step a).

In some aspects, the biomass and first microbial inoculant composition can be introduced into the first reactor vessel at the same time. In some aspects, the biomass can be introduced into the first reactor vessel before or after the first microbial inoculant composition is introduced into the first reactor vessel. In some aspects, the biomass and second microbial inoculant composition can introduced into the second reactor vessel at the same time. In some aspects, the biomass can be introduced into the second reactor vessel before or after the second microbial inoculant composition is introduced into the second reactor vessel.

In some aspects, the biomass can be a feedstock, a plant material, an animal material, food, water, industrial waste or organic waste products, residual waste thereof, or combination thereof. In some aspects, the biomass can be any feedstock that can be digested biologically (e.g., breaking it down into smaller molecules). In some aspects, the feedstock can be pretreated. In some aspects, the feedstock can be pretreated to enhance digestibility. Examples of pretreatment approaches include but are not limited to chemical (e.g., alkaline, acidic and inorganic salts), physical (e.g., microwaves and liquid hot water), and biological (e.g., enzymatic and fungal). In some aspects, the biomass can pretreated with a microbial inoculant composition. In some aspects, the microbial inoculant composition can comprise a first bacterial strain and a second bacterial strain. In some aspects, the first bacterial strain can comprise Clostridium spp. In some aspects, the 16S sequence of Clostridium spp. can comprise any one of the Clostridium spp. listed in Table 1 or Table 2. In some aspects, the second bacterial strain can comprise an aquatic Pseudomonas spp. bacteria with a 16S nucleic acid sequence that is at least about 97% identical to any one of the Pseudomonas spp. bacteria listed in Table 1 or Table 2. In some aspects, the first microbial inoculant can comprise one or more of the microbes listed in Table 1, Table 2 or Table 3. In some aspects, the first microbial inoculant can comprise at least one different microbial strain, wherein the 16S sequence of the one different microbial strain can comprise a 16S sequence that is at least about 97% identical to one or more of the 16S sequences listed in Table 1, Table 2 or Table 3.

In some aspects, the method can comprise maintaining the ORP. In some aspects, the ORP can be maintained in step a) between around −50 mV and −600 mV. In some aspects, the ORP can be maintained in step c) between around −100 mV and less than 1000 mV. In some aspects, the ORP can be maintained in step c) between around −300 mV and less than-400 mV. In some aspects, ORP can be be maintained at a negative ORP. In some aspects, the ORP of the first reactor vessel can be less than −50 mV. In some aspects, the ORP of the second reactor vessel can be between less than 0 and greater than −150 mV. In some aspects, the method can further comprise maintaining the ORP in the third reactor vessel between around −80 mV and −800 mV. In some aspects, the ORP of the third reactor vessel can be zero or below zero. In some aspects, oxygen can removed or introduced into one or more of the reactor vessels. In some aspects, oxygen can be introduced into a reactor vessel in the form of an oxygen-containing gas. In some aspects, the oxygen-containing gas can be pure oxygen, a blend of oxygen and inert gas(es) or an oxygen liberating source (e.g., hydrogen peroxide). For example, if the ORP is too high to produce hydrogen, the one or more microbes may produce carbon dioxide, thereby reducing the ORP, and oxygen can be introduced (e.g., continuous oxygen containing air flow) into a reactor vessel to prevent the ORP from being further reduced and stopping methane production.

In some aspects, the methods can further comprise maintaining a pH level of the contents of the first reactor vessel at a first pH level or within a first pH range. In some aspects, the first pH level can be less than 6 or the first pH range can be between 1 and 6. In some aspects, the pH of the first reactor vessel can be less than 5. In some aspects, the pH of the first reactor vessel can be 4, 3, 2 or 1. In some aspects, the pH of the second reactor vessel can be between 1 and 8. In some aspects, the pH of the second reactor vessel can be between 2 and 6. In some aspects, the first pH level of the third reactor vessel can be less than 6 or the first pH range can be between 1 and 6. In some aspects, the pH of the third reactor vessel can be less than 5. In some aspects, the pH of the third reactor vessel can be 4, 3, 2 or 1.

In some aspects, the methods can further comprise maintaining the temperature of the contents of the first reactor vessel at above 0° C. In some aspects, the temperature of the first reactor vessel can be maintained between 65° F. and 135° F. In some aspects, the methods can further comprise maintaining the temperature of the contents of the second reactor vessel at above 0° C. In some aspects, the temperature of the second reactor vessel can be maintained between 65° F. and 135° F. In some aspects, the methods can further comprise maintaining the temperature of the contents of the third reactor vessel at above 0° C. In some aspects, the temperature of the third reactor vessel can be maintained between 65° F. and 135° F.

In some aspects, the biogas collected from the first reactive vessel can be hydrogen. In some aspects, the biogas collected from the second reactive vessel can be methane, hydrogen sulfide, carbon dioxide, ammonia, NO₂, or other low molecular weight volatile organic carbons (VOCs). For example, the first reactor vessel and the third reactor vessel can produce hydrogen, some carbon dioxide and some methane as well as a small portion of NO₂, and ammonia, and the second reactor vessel can produce methane, carbon dioxide, ammonia, hydrogen sulfide, NO₂, and other trace gases.

In some aspects, the biomass in the third reactor vessel can be separated into a solid portion and a liquid portion. In some aspects, the solid portion of the biomass can be separated into primitive carbon(s). In some aspects, the liquid portion comprises inorganic plant nutrients. In some aspects, the total amounts of inorganic plant nutrients in the liquid portion can be increased. In some aspects, the overall amount of the solid portion can be decreased. In some aspects, the methods can further comprise collecting a portion of the liquid portion from the third reactor vessel. In some aspects, the liquid portion can will be high in nutrients. In some aspects, the liquid portion can be used as a fertilizer. In some aspects, the liquid portion can provide solubilized metals (e.g., nickel, iron, manganese, boron, cobalt, and lithium) that can be extracted for their value. In some aspects, the methods can further comprise collecting a portion of the solid portion from the third reactor vessel. In some aspects, the solid portion collected can be reused on the field as a compost source. In some aspects, the solid portion can be used as a fertilizer. In some aspects, the solid portion can be used as a biomass. In some aspects, the solid portion can contain primarily metals and lignin- and silica-based components. In some aspects, the solid portion can be further refined to create a granular fertilizer, abrasive, carbon source material for construction. In some aspects, the metals can be further refined for use in industry.

In some aspects, solids or liquids from an anaerobic lagoon or a manure lagoon can be used as a source of the biomaterial or biomass used in any of the bioreactor vessels described herein. In some aspects, the anaerobic lagoon or the manure lagoon itself can serve as a bioreactor vessel. In aspects, the methods can comprise the steps of: a) applying a composition comprising two or more bacterial strains disclosed herein to an anaerobic lagoon or a manure lagoon. In some aspects, a first bacterial strain can comprise Clostridium spp. In some aspects, the 16S sequence of Clostridium spp. can comprise any one of the Clostridium spp. listed in Table 1 or Table 2. In some aspects, a second bacterial strain can comprise an aquatic Pseudomonas spp. bacteria with a 16S nucleic acid sequence that is at least about 97% identical to any one of the Pseudomonas spp. bacteria listed in Table 1 or Table 2.

### Methods for Selectively and Separately Producing Hydrogen and Methane II

Disclosed herein are methods for selectively and separately producing hydrogen and methane.

Disclosed herein are methods for selectively and separately producing hydrogen and methane, the methods comprising: a) contacting a biomass in a first reactor vessel with a first microbial inoculant composition under anaerobic conditions to facilitate the digestion of the biomass to produce a digested biomass, wherein the first reactor vessel is maintained at a first oxidation reduction potential (ORP); b) collecting hydrogen gas from the first reactor vessel and transferring a portion of the digested biomass from step a) to a second reactor vessel; c) introducing an oxygen-containing gas to the second reactor vessel to change the first ORP from the first reactor vessel to a second ORP in the second reactor vessel and contacting the digested biomass in the second reactor vessel with a second microbial inoculant composition under aerobic conditions to facilitate the digestion of the digested biomass; and d) collecting biogas from the second reactor vessel, e) collecting a portion of the digested biomass from step a) and separating a liquid fraction from a solid fraction of the digested biomass, f) transferring the solid fraction of step e) into the first or second reactor vessel or both the first and second reactor vessels, g) transferring the liquid fraction or supernatant of step e) into a moving biofilm bed reactor (MBBR), contacting the liquid fraction in the MBBR with a microbial inoculant composition similar or the same as the content of the microbial inoculant composition used in the second reactor vessel; h) digesting the liquid fraction in the MBBR under conditions to remove one or more organic acids (e.g., acetate) from the liquid fraction to produce a liquid fraction with a reduced one or more organic acid (e.g., acetate) content; and i) optionally, transferring the liquid fraction or supernatant with a reduced one or more organic acid (e.g., acetate) content of step h) into the first reactor vessel, wherein the microbial inoculant comprising comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2, wherein the second microbial inoculant comprises one or more methanogen producers.

In some aspects, the methods can comprise: a) contacting a biomass in a first reactor vessel with a first microbial inoculant composition under anaerobic conditions to facilitate the digestion of the biomass to produce a digested biomass. In some aspects, the first reactor vessel can be maintained at a first oxidation reduction potential (ORP). In some aspects, the methods comprise producing a partially digested biomass. In some aspects, the methods can comprise b) collecting hydrogen gas from the first reactor vessel and transferring a portion of the digested biomass or the partially digested biomass from step a) to a second reactor vessel. In some aspects, the methods can comprise c) introducing an oxygen-containing gas to the second reactor vessel to change the first ORP from the first reactor vessel to a second ORP in the second reactor vessel and contacting the digested biomass or partially digested biomass in the second reactor vessel with a second microbial inoculant composition under anaerobic conditions to facilitate the digestion of the digested biomass or partially digested biomass. In some aspects, the step of introducing an oxygen-containing gas to the second reactor vessel to change the first ORP from the first reactor vessel to a second ORP in the second reactor vessel and contacting the digested biomass in the second reactor vessel with a second microbial inoculant composition can be carried out under aerobic conditions allowing it to reduce to an anaerobic conditions to facilitate the digestion of the digested biomass. In some aspects, the methods can comprise d) collecting biogas from the second reactive vessel. In some aspects, the methods can comprise e) collecting a portion of the digested or partially digested biomass from step a) and separating a liquid portion from a solid fraction from the digested or partially digested biomass. In some aspects, the methods can comprise e) collecting a portion of the digested or partially digested biomass from step a) and separating a liquid portion from a solid fraction from the digested or partially digested biomass, thereby producing a supernatant. In some aspects, the supernatant can comprise acetate and other organic acids. In some aspects, the supernatant comprising acetate and other organic acids can be added to a MBBR. In some aspects, the methods can further comprise f) transferring the solid fraction of step e) into the first or second reactor vessel or both the first and second reactor vessels. In some aspects, the methods an comprise g) transferring the liquid fraction or supernatant of step e) into a moving biofilm bed reactor (MBBR), contacting the liquid fraction in the MBBR with a microbial inoculant composition similar or the same as the content of the microbial inoculant composition used in the second reactor vessel. For example, the microbial inoculant composition used in the MBBR can comprise the same bacterial strains as the first microbial inoculant composition or can comprise 50%, 60%, 70%, 80%, 90%, 99%, or any percentage in between of the bacterial strains as the first microbial inoculant composition. In some aspects, the methods can comprise h) digesting the liquid fraction in the MBBR under conditions to remove one or more organic acids (e.g., acetate) from the liquid fraction to produce a liquid fraction with a reduced acetate content. In some aspects, the methods can comprise h) digesting the liquid fraction in the MBBR under conditions to remove one or more organic acids from the liquid fraction to produce a liquid fraction with a reduced acetate content and a biogas. In some aspects, the biogas can be a mixture of methane and CO₂. In some aspects, the methods can optionally comprise i) transferring the liquid fraction or supernatant with a reduced organic acid (e.g., acetate) content of step h) into the first reactor vessel. In some aspects, the first reactor vessel can comprise a first microbial inoculant composition. In some aspects, the methods can further comprise collecting biogas from the MBBR. In some aspects, the biogas collected from the MBBR can be methane and/or CO₂ or a mixture thereof. In some aspects, the biogas collected from the MBBR can be methane, hydrogen, ammonia, CO₂, hydrogen sulfide, N₂O or any combination thereof. In some aspects, the methods can comprise the step of transferring a portion of the digested or partially digested biomass from the first or second reactor vessel to a third reactor vessel and contacting the digested or partially digested biomass in the third reactor vessel with a third microbial inoculant composition to facilitate the digestion of the biomass under conditions to convert ammonia into nitrates. In some aspects, the methods can further comprise collecting liquids from the third reactor vessel. In some aspects, the method can be continuous. In some aspects, biomass can be continuously added or sporadically added to the first reactor vessel.

In some aspects, the first microbial inoculant composition can comprise a first bacterial strain and a second bacterial strain, wherein the first bacterial strain comprises Clostridium spp. In some aspects, the 16S sequence of Clostridium spp. can comprise any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2.

In some aspects, the first microbial inoculant composition in step a) can decrease or suppress one or more methanogens in the first reactor vessel. In some aspects, the first microbial inoculant composition can create or change the environment in the first reactor vessel such than the metabolism of any of methanogens present in the first reactor vessel is suppressed.

In some aspects, the second microbial inoculant composition can be different than the first microbial inoculant composition. In some aspects, the second microbial inoculant composition can comprise one or more methanogen producers. Examples of methanogoen producers include but are not limited to Methanobacterium bryantii, Methanobacterium formicum, Methanobrevibacter arboriphilicus, Methanobrevibacter gottschalkii, Methanobrevibacter ruminantium, Methanobrevibacter smithii, Methanococcus chunghsingensis, Methanococcus burtonii, Methanococcus aeolicus, Methanococcus deltae, Methanococcus jannaschii, Methanococcus maripaludis, Methanococcus vannielii, Methanocorpusculum labreanum, Methanoculleus bourgensis (Methanogenium olentangyi and Methanogenium bourgense), Methanoculleus marisnigri, Methanoflorens stordalenmirensis, Methanofollis liminatans, Methanogenium cariaci, Methanogenium frigidum, Methanogenium organophilum, Methanogenium wolfei, Methanomicrobium mobile, Methanopyrus kandleri, Methanoregula boonei, Methanosaeta concilii, Methanosaeta thermophile, Methanosarcina acetivorans, Methanosarcina barkeri, Methanosarcina mazei, Methanosphaera stadtmanae, Methanospirillium hungatei, Methanothermobacter defluvii (Methanobacterium defluvii), Methanothermobacter thermautotrophicus (Methanobacterium thermoautotrophicum), Methanothermobacter thermoflexus (Methanobacterium thermoflexum), Methanothermobacter wolfei (Methanobacterium wolfei), and Methanothrix sochngenii.

In some aspects, the third microbial inoculant composition can comprise a first bacterial strain and a second bacterial strain. In some aspects, the first bacterial strain can comprise Clostridium spp. In some aspects, the 16S sequence of Clostridium spp. can comprise any one of the Clostridium spp. listed in Table 1 or Table 2. In some aspects, the second bacterial strain can comprise an aquatic Pseudomonas spp. bacteria with a 16S nucleic acid sequence that is at least about 97% identical to any one of the Pseudomonas spp. bacteria listed in Table 1 or Table 2. In some aspects, the third microbial inoculant composition can be the same as the first microbial inoculant composition in step a).

In some aspects, the MBBR can comprise a microbial inoculant composition. In some aspects, the microbial inoculant composition can be the same microbial inoculant composition present in the second reactor vessel.

In some aspects, the biomass and first microbial inoculant composition can be introduced into the first reactor vessel at the same time. In some aspects, the biomass can be introduced into the first reactor vessel before or after the first microbial inoculant composition is introduced into the first reactor vessel. In some aspects, the biomass and second microbial inoculant composition can introduced into the second reactor vessel at the same time. In some aspects, the biomass can be introduced into the second reactor vessel before or after the second microbial inoculant composition is introduced into the second reactor vessel.

In some aspects, the biomass can be a feedstock, a plant material, an animal material, food, water, industrial waste or organic waste products, residual waste thereof, or combination thereof. In some aspects, the feedstock can be pretreated. In some aspects, the feedstock can be pretreated to enhance digestibility. Examples of pretreatment approaches include but are not limited to chemical (e.g., alkaline, acidic and inorganic salts), physical (e.g., microwaves and liquid hot water), and biological (e.g., enzymatic and fungal). In some aspects, the biomass can pretreated with a microbial inoculant composition. In some aspects, the microbial inoculant composition can comprise a first bacterial strain and a second bacterial strain. In some aspects, the first bacterial strain can comprise Clostridium spp. In some aspects, the 16S sequence of Clostridium spp. can comprise any one of the Clostridium spp. listed in Table 1 or Table 2. In some aspects, the second bacterial strain can comprise an aquatic Pseudomonas spp. bacteria with a 16S nucleic acid sequence that is at least about 97% identical to any one of the Pseudomonas spp. bacteria listed in Table 1 or Table 2. In some aspects, the first microbial inoculant can comprise one or more of the microbes listed in Table 1, Table 2 or Table 3. In some aspects, the first microbial inoculant can comprise at least one different microbial strain, wherein the 16S sequence of the one different microbial strain can comprise a 16S sequence that is at least about 97% identical to one or more of the 16S sequences listed in Table 1, Table 2 or Table 3.

In some aspects, the method can comprise maintaining the MBBR at an ORP that can be similar or the same ORP as the second ORP in the second reactor vessel. In some aspects, the ORP can be maintained in step a) between around −50 mV and −600 mV. In some aspects, the ORP can be maintained in step c) between around −100 mV and less than 1000 mV. In some aspects, the ORP can be maintained in step c) between around −300 mV and less than −400 mV.

In some aspects, ORP can be be maintained at a negative ORP. In some aspects, the ORP of the first reactor vessel can be less than −50 mV. In some aspects, the ORP of the second reactor vessel can be between less than 0 and greater than −150 mV. In some aspects, the method can further comprise maintaining the ORP in the third reactor vessel between around −80 mV and −800 mV. In some aspects, the ORP of the third reactor vessel can be zero or below.

In some aspects, the oxygen-containing gas can be pure oxygen, a blend of oxygen and inert gas(es) or an oxygen liberating source (e.g., hydrogen peroxide).

In some aspects, the methods can further comprise maintaining a pH level of the contents of the first reactor vessel at a first pH level or within a first pH range. In some aspects, the first pH level can be less than 6 or the first pH range can be between 1 and 6. In some aspects, the pH of the first reactor vessel can be less than 5. In some aspects, the pH of the first reactor vessel can be 4, 3, 2 or 1. In some aspects, the pH of the second reactor vessel can be between 1 and 8. In some aspects, the pH of the second reactor vessel can be between 2 and 6. In some aspects, the first pH level of the third reactor vessel can be less than 6 or the first pH range can be between 1 and 6. In some aspects, the pH of the third reactor vessel can be less than 5. In some aspects, the pH of the third reactor vessel can be 4, 3, 2 or 1.

In some aspects, the methods can further comprise maintaining the temperature of the contents of the first reactor vessel at above 0° C. In some aspects, the temperature of the first reactor vessel can be maintained between 65° F. and 135° F. In some aspects, the methods can further comprise maintaining the temperature of the contents of the second reactor vessel at above 0° C. In some aspects, the temperature of the second reactor vessel can be maintained between 65° F. and 135° F. In some aspects, the methods can further comprise maintaining the temperature of the contents of the third reactor vessel at above 0° C. In some aspects, the temperature of the third reactor vessel can be maintained between 65° F. and 135° F.

In some aspects, the biogas collected from the second reactor vessel can be methane.

In some aspects, the biomass in the third reactor vessel can be separated into a solid portion and a liquid portion. In some aspects, the solid portion of the biomass can be separated into primitive carbon(s). In some aspects, the liquid portion comprises inorganic plant nutrients. In some aspects, the total amounts of inorganic plant nutrients in the liquid portion can be increased. In some aspects, the overall amount of the solid portion can be decreased. In some aspects, the methods can further comprise collecting a portion of the liquid portion from the third reactor vessel. In some aspects, the liquid portion can be used as a fertilizer. In some aspects, the methods can further comprise collecting a portion of the solid portion from the third reactor vessel. In some aspects, the solid portion collected can be reused on the field as a compost source. In some aspects, the solid portion can be used as a fertilizer. In some aspects, the solid portion can be used as a biomass.

In some aspects, the amount of acetate that can be reduced in the liquid fraction can be at least 5% relative prior to the digesting step. In some aspects, the amount of the acetate that is reduced is between 5% and 99% relative prior to the digesting step. In some aspects, the amount of the acetate that is reduced is at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or any percent decrease in between relative prior to the digesting step.

Methods for Selectively Producing Hydrogen from a Landfill Leachate

Disclosed herein are methods for selectively producing hydrogen from a landfill leachate. In some aspects, the methods can comprise the steps of: a) applying a composition comprising two or more bacterial strains to the landfill leachate. In some aspects, a first bacterial strain can comprise Clostridium spp. In some aspects, the 16S sequence of Clostridium spp. can comprise any one of the Clostridium spp. listed in Table 1 or Table 2. In some aspects, a second bacterial strain can comprise an aquatic Pseudomonas spp. bacteria with a 16S nucleic acid sequence that is at least about 97% identical to any one of the Pseudomonas spp. bacteria listed in Table 1 or Table 2. In some aspects, additional microbes can be added in combination with two or more bacterial strains disclosed herein, such as plastic eating microbes (e.g., Ideonella sakaiensis) to breaking down or consuming plastic (e.g. plastic polyethylene terephthalate (PET) using it as both a carbon and energy source) present in the landfill. In some aspects, the methods can comprise b) collecting samples from the landfill leachate. In some aspects, the methods can comprise introducing the landfill leachate sample into a first reactor vessel and contacting the landfill leachate sample with the microbial inoculant composition in step a) under anaerobic conditions to facilitate the digestion of the landfill leachate sample. In some aspects, the first reactor vessel can be maintained at a first oxidation reduction potential (ORP). In some aspects, the digestion of the landfill leachate sample can be full or partial. In some aspects, the methods can comprise d) collecting hydrogen gas from the first reactor vessel and transferring a portion of the digested or partially digested landfill leachate sample from step c) to a second reactor vessel. In some aspects, the methods can comprise e) introducing an oxygen-containing gas to the second reactor vessel to change the first ORP from the first reactor vessel to a second ORP in the second reactor vessel and contacting the digested or partially digested landfill leachate sample in the second reactor vessel with a second microbial inoculant composition under aerobic conditions to facilitate the digestion or partial digestion of the landfill leachate sample. In some aspects, the methods can comprise h) transferring a portion of the solid fraction of step g) into the first or second reactor vessel or both the first and second reactor vessels. In some aspects, the methods can comprise i) transferring the liquid fraction or supernatant of step g) into a moving biofilm bed reactor (MBBR), contacting the liquid fraction in the MBBR with a microbial inoculant composition similar or the same as the content of the microbial inoculant composition used in the second reactor vessel. In some aspects, the methods can comprise j) digesting the liquid fraction in the MBBR under conditions to remove acetate from the liquid fraction to produce a liquid fraction with a reduced acetate content. In some aspects, the methods can comprise j) digesting the liquid fraction in the MBBR under conditions to remove acetate from the liquid fraction to produce a liquid fraction with a reduced acetate content and a biogas. In some aspects, the biogas can be a mixture of methane and CO₂. In some aspects, the biogas collected from the MBBR can be methane, hydrogen, ammonia, CO₂, hydrogen sulfide, N₂O or any combination thereof. In some aspects, the methods can optionally comprise k) transferring the liquid fraction or supernatant with a reduced acetate content of step h) into the first reactor vessel. In some aspects, the first reactor vessel can comprise a first microbial inoculant composition. In some aspects, the methods can further comprise collecting biogas from the MBBR. In some aspects, the biogas collected from the MBBR can be methane and/or CO₂ or a mixture thereof. In some aspects, the biogas collected from the MBBR can be methane, hydrogen, ammonia, CO₂, hydrogen sulfide, N₂O or any combination thereof.

In some aspects, the methods can further comprise the step of transferring a portion of the digested or partially digested landfill leachate from the first or second reactor vessel to a third reactor vessel and contacting the digested or partially digested landfill leachate in the third reactor vessel with a third microbial inoculant composition to facilitate the digestion of the landfill leachate under conditions to convert ammonia into nitrates. In some aspects, the methods can further comprise collecting liquids from the third reactor vessel. In some aspects, the method can be continuous. In some aspects, biomass can be continuously added or sporadically added to the first reactor vessel.

In some aspects, the methods disclosed herein can comprise collecting samples from the anaerobic lagoon or the manure lagoon. In some aspects, after applying a composition comprising two or more bacterial strains disclosed herein to an anaerobic lagoon or a manure lagoon, the methods can further comprise collecting a portion of a liquid or solid portion from the anaerobic lagoon or the manure lagoon.

In some aspects, the methods disclosed herein can comprise applying a composition comprising two or more bacterial strains disclosed herein to a landfill. In some aspects, the application of the composition comprising two or more bacterial strains disclosed herein to a landfill can raise the liquid for further application(s) to produce hydrogen or methane or mine one or more compounds present in the landill (e.g. metals).

In some aspects, methods disclosed herein can comprise applying a composition comprising two or more bacterial strains disclosed herein to a landfill, wherein the landfill comprises a liner. In some aspects, the liner can be a natural liner that can be suitable for use in the landfill. In some aspects, the landfill liner can be a clay liner. In some aspects, the landfill liner can be used to create the first reactor vessel. Thus, in some aspects, the first reactor vessel can be a natural liner that can be suitable for use in the landfill. In some aspects, the landfill liner that is the first reactor vessel can be used in the methods disclosed herein to produce hydrogen gas. The use of the composition comprising two or more bacterial strains, wherein a first bacterial strain comprises Clostridium spp., and wherein the 16S sequence of Clostridium spp. comprises any one of the Clostridium spp. listed in Table 1 or Table 2 and the second bacterial strain comprising an aquatic Pseudomonas spp. bacteria with a 16S nucleic acid sequence that is at least about 97% identical to any one of the Pseudomonas spp. bacteria listed in Table 1 or Table 2 will force the ORP and pH to be lower and the pH to become sufficiently acidic such that one or more metals present in the landfill will be soluble and one or more other microbes that are present (before or after the application of the compositions disclosed herein) can break the one or more metals down further, and the leachate can be obtained or isolated and used to make methane or to extract the one or more metals present in the first reactor vessel. The gas produced in the landfill at this step of the method can be hydrogen. In some aspects, the hydrogen can be captured or burned on site. In some aspects, the leachate can be removed to produce methane using one or more of the methods disclosed herein.

In some aspects, the landfill can be used as the first reactor vessel, and used to produce hydrogen using one or more of the methods disclosed herein, and the organic acids can be converted to acetate without producing methane using one or more of the methods disclosed herein.

In some aspects, the liquid or solid portion can be used as a fertilizer. In some aspects, the methods can further comprise collecting a portion of the solid portion from the anaerobic lagoon or the manure lagoon. In some aspects, the solid portion collected can be reused on the field as a compost source. In some aspects, the solid portion can be used as a fertilizer. In some aspects, the solid portion can be used as a biomass.

In some aspects, the disclosed compositions (e.g. the microbial inoculant compositions disclosed herein) can be used to change or create or provide an environment that supports the availability of nutrition in a solubilized form that can be removed or collected in a liquid fraction of a sample. In some aspects, the disclosed compositions when added to a sample can result in an environment that preferentially forms nitrogen as NO₃. In some aspects, the presence of nitrogen in the anaerobic lagoon or a manure lagoon or a pond can act as a fertilizer, thereby reducing the volatilization of the nitrogen as ammonia or ammonium. In some aspects, the nitrogen can be evaporated and used to make a concentrated fertilizer with the NO₃. For example, disclosed are methods comprising applying a composition comprising two or more bacterial strains disclosed herein to an anaerobic lagoon or a manure lagoon, wherein the two or more bacterial strains increases the availability of nutrition in a solubilized form that can be removed in a liquid fraction of the anaerobic lagoon or a manure lagoon or can preferentially form nitrogen in the form of NO₃. Such methods can result in an increase in nitrogen in the anaerobic lagoon or a manure lagoon or can reduce volatilization of nitrogen as ammonia after application to the anaerobic lagoon or a manure lagoon. In some aspects, water can be removed from a portion of the anaerobic lagoon or a manure lagoon to create a concentrated fertilizer enriched in NO₃.

In some aspects, the first microbial inoculant composition can comprise a first bacterial strain and a second bacterial strain, wherein the first bacterial strain comprises Clostridium spp. In some aspects, the 16S sequence of Clostridium spp. can comprise any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2.

In some aspects, the first microbial inoculant composition in step a) can decrease or suppress one or more methanogens in the first reactor vessel. In some aspects, the first microbial inoculant composition can create or change the environment in the first reactor vessel such than the metabolism of any of methanogens present in the first reactor vessel is suppressed.

In some aspects, the second microbial inoculant composition can be different than the first microbial inoculant composition. In some aspects, the second microbial inoculant composition can comprise one or more methanogen producers. Examples of methanogoen producers include but are not limited to Methanobacterium bryantii, Methanobacterium formicum, Methanobrevibacter arboriphilicus, Methanobrevibacter gottschalkii, Methanobrevibacter ruminantium, Methanobrevibacter smithii, Methanococcus chunghsingensis, Methanococcus burtonii, Methanococcus aeolicus, Methanococcus deltae, Methanococcus jannaschii, Methanococcus maripaludis, Methanococcus vannielii, Methanocorpusculum labreanum, Methanoculleus bourgensis (Methanogenium olentangyi and Methanogenium bourgense), Methanoculleus marisnigri, Methanoflorens stordalenmirensis, Methanofollis liminatans, Methanogenium cariaci, Methanogenium frigidum, Methanogenium organophilum, Methanogenium wolfei, Methanomicrobium mobile, Methanopyrus kandleri, Methanoregula boonei, Methanosaeta concilii, Methanosaeta thermophile, Methanosarcina acetivorans, Methanosarcina barkeri, Methanosarcina mazei, Methanosphaera stadtmanae, Methanospirillium hungatei, Methanothermobacter defluvii (Methanobacterium defluvii), Methanothermobacter thermautotrophicus (Methanobacterium thermoautotrophicum), Methanothermobacter thermoflexus (Methanobacterium thermoflexum), Methanothermobacter wolfei (Methanobacterium wolfei), and Methanothrix sochngenii.

In some aspects, the third microbial inoculant composition can comprise a first bacterial strain and a second bacterial strain. In some aspects, the first bacterial strain can comprise Clostridium spp. In some aspects, the 16S sequence of Clostridium spp. can comprise any one of the Clostridium spp. listed in Table 1 or Table 2. In some aspects, the second bacterial strain can comprise an aquatic Pseudomonas spp. bacteria with a 16S nucleic acid sequence that is at least about 97% identical to any one of the Pseudomonas spp. bacteria listed in Table 1 or Table 2. In some aspects, the third microbial inoculant composition can be the same as the first microbial inoculant composition in step a).

In some aspects, the landfill leachate can comprise feedstock, a plant material, an animal material, food, water, industrial waste or organic waste products, residual waste thereof, or combination thereof. In some aspects, the landfill leachate can be pretreated. In some aspects, the landfill leachate can be pretreated to enhance digestibility. Examples of pretreatment approaches include but are not limited to chemical (e.g., alkaline, acidic and inorganic salts), physical (e.g., microwaves and liquid hot water), and biological (e.g., enzymatic and fungal). In some aspects, the landfill leachate can pretreated with a microbial inoculant composition. In some aspects, the microbial inoculant composition can comprise a first bacterial strain and a second bacterial strain. In some aspects, the first bacterial strain can comprise Clostridium spp. In some aspects, the 16S sequence of Clostridium spp. can comprise any one of the Clostridium spp. listed in Table 1 or Table 2. In some aspects, the second bacterial strain can comprise an aquatic Pseudomonas spp. bacteria with a 16S nucleic acid sequence that is at least about 97% identical to any one of the Pseudomonas spp. bacteria listed in Table 1 or Table 2. In some aspects, the first microbial inoculant can comprise one or more of the microbes listed in Table 1, Table 2 or Table 3. In some aspects, the first microbial inoculant can comprise at least one different microbial strain, wherein the 16S sequence of the one different microbial strain can comprise a 16S sequence that is at least about 97% identical to one or more of the 16S sequences listed in Table 1, Table 2 or Table 3.

In some aspects, the landfill leachate and the first microbial inoculant composition can be introduced into the first reactor vessel at the same time. In some aspects, the landfill leachate can be introduced into the first reactor vessel before or after the first microbial inoculant composition is introduced into the first reactor vessel. In some aspects, the landfill leachate and second microbial inoculant composition can introduced into the second reactor vessel at the same time. In some aspects, the landfill leachate can be introduced into the second reactor vessel before or after the second microbial inoculant composition is introduced into the second reactor vessel.

In some aspects, the MBBR can comprise a microbial inoculant composition. In some aspects, the microbial inoculant composition can be the same microbial inoculant composition present in the second reactor vessel.

In some aspects, the method can comprise maintaining the MBBR at an ORP that can be similar or the same ORP as the second ORP in the second reactor vessel. In some aspects, the ORP can be maintained in step a) between around −50 mV and −600 mV. In some aspects, the ORP can be maintained in step c) between around −100 mV and less than 1000 mV. In some aspects, the ORP can be maintained in step c) between around −300 mV and less than −400 mV. In some aspects, ORP can be be maintained at a negative ORP. In some aspects, the ORP of the first reactor vessel can be less than −50 mV. In some aspects, the ORP of the second reactor vessel can be between less than 0 and greater than −150 mV. In some aspects, the method can further comprise maintaining the ORP in the third reactor vessel between around −80 mV and −800 mV. In some aspects, the ORP of the third reactor vessel can be zero or below.

In some aspects, the oxygen-containing gas can be pure oxygen, a blend of oxygen and inert gas(es) or an oxygen liberating source (e.g., hydrogen peroxide).

In some aspects, the methods can further comprise maintaining a pH level of the contents of the first reactor vessel at a first pH level or within a first pH range. In some aspects, the first pH level can be less than 6 or the first pH range can be between 1 and 6. In some aspects, the pH of the first reactor vessel can be less than 5. In some aspects, the pH of the first reactor vessel can be 4, 3, 2 or 1. In some aspects, the pH of the second reactor vessel can be between 1 and 8. In some aspects, the pH of the second reactor vessel can be between 2 and 6. In some aspects, the first pH level of the third reactor vessel can be less than 6 or the first pH range can be between 1 and 6. In some aspects, the pH of the third reactor vessel can be less than 5. In some aspects, the pH of the third reactor vessel can be 4, 3, 2 or 1.

In some aspects, the methods can further comprise maintaining the temperature of the contents of the first reactor vessel at above 0° C. In some aspects, the temperature of the first reactor vessel can be maintained between 65° F. and 135° F. In some aspects, the methods can further comprise maintaining the temperature of the contents of the second reactor vessel at above 0° C. In some aspects, the temperature of the second reactor vessel can be maintained between 65° F. and 135° F. In some aspects, the methods can further comprise maintaining the temperature of the contents of the third reactor vessel at above 0° C. In some aspects, the temperature of the third reactor vessel can be maintained between 65° F. and 135° F.

In some aspects, the landfill leachate in the third reactor vessel can be separated into a solid portion and a liquid portion. In some aspects, the solid portion of the landfill leachate can be separated into primitive carbon(s). In some aspects, the liquid portion comprises inorganic plant nutrients. In some aspects, the total amounts of inorganic plant nutrients in the liquid portion can be increased. In some aspects, the overall amount of the solid portion can be decreased. In some aspects, the methods can further comprise collecting a portion of the liquid portion from the third reactor vessel. In some aspects, the liquid portion can be used as a fertilizer. In some aspects, the methods can further comprise collecting a portion of the solid portion from the third reactor vessel. In some aspects, the solid portion collected can be reused on the field as a compost source. In some aspects, the solid portion can be used as a fertilizer. In some aspects, the solid portion can be used as a biomass.

In some aspects, the amount of acetate that can be reduced in the liquid fraction can be at least 5% relative prior to the digesting step. In some aspects, the amount of the acetate that is reduced is between 5% and 99% relative prior to the digesting step. In some aspects, the amount of the acetate that is reduced is at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or any percent decrease in between relative prior to the digesting step.

### Methods of Producing Fertilizers and Uses Thereof

Disclosed herein are methods of producing fertilizers enriched in organic nitrogen. In some aspects, the methods can comprise using one or more of the methods disclosed herein. As disclosed herein, the methods of selectively producing hydrogen and selectively producing methane can generate liquids and solids. The liquids and solids produced by any of these disclosed methods can be used as a fertilizer.

Also disclosed herein are methods of increasing plant growth using the fertilizers disclosed herein. In some aspects, the methods can comprise applying a fertilizer enriched in organic nitrogen. In some aspects, the methods can comprise applying a fertilizer enriched in organic nitrogen to a seed, a field or a plant.

Further disclosed herein are methods for selectively enriching organic nitrogen from a landfill leachate. In the some aspects, the methods can comprise the steps of: a) applying a composition comprising two or more bacterial strains to the landfill leachate. In some aspects, a first bacterial strain can comprise Clostridium spp. In some aspects, the 16S sequence of Clostridium spp. can comprise any one of the Clostridium spp. listed in Table 1 or Table 2. In some aspects, a second bacterial strain can comprise an aquatic Pseudomonas spp. bacteria with a 16S nucleic acid sequence that is at least about 97% identical to any one of the Pseudomonas spp. bacteria listed in Table 1 or Table 2.

In some aspects, the methods can comprise b) collecting samples from the landfill leachate. In some aspects, the methods can comprise introducing the landfill leachate sample into a first reactor vessel and contacting the landfill leachate sample with the microbial inoculant composition in step a) under anaerobic conditions to facilitate the digestion of the landfill leachate sample. In some aspects, the first reactor vessel can be maintained at a first oxidation reduction potential (ORP). In some aspects, the digestion of the landfill leachate sample can be full or partial. In some aspects, the methods can comprise d) collecting hydrogen gas from the first reactor vessel and transferring a portion of the digested or partially digested landfill leachate sample from step c) to a second reactor vessel. In some aspects, the methods can comprise e) introducing an oxygen-containing gas to the second reactor vessel to change the first ORP from the first reactor vessel to a second ORP in the second reactor vessel and contacting the digested or partially digested landfill leachate sample in the second reactor vessel with a second microbial inoculant composition under aerobic conditions to facilitate the digestion or partial digestion of the landfill leachate sample. In some aspects, the methods can comprise h) transferring a portion of the solid fraction of step g) into the first or second reactor vessel or both the first and second reactor vessels. In some aspects, the methods can comprise i) transferring the liquid fraction or supernatant of step g) into a moving biofilm bed reactor (MBBR), contacting the liquid fraction in the MBBR with a microbial inoculant composition similar or the same as the content of the microbial inoculant composition used in the second reactor vessel. In some aspects, the methods can comprise j) digesting the liquid fraction in the MBBR under conditions to remove acetate from the liquid fraction to produce a liquid fraction with a reduced acetate content. In some aspects, the methods can comprise j) digesting the liquid fraction in the MBBR under conditions to remove acetate from the liquid fraction to produce a liquid fraction with a reduced acetate content and a biogas. In some aspects, the biogas can be a mixture of methane and CO₂. In some aspects, the biogas collected from the MBBR can be methane, hydrogen, ammonia, CO₂, hydrogen sulfide, N₂O or any combination thereof. In some aspects, the methods can optionally comprise k) transferring the liquid fraction or supernatant with a reduced acetate content of step h) into the first reactor vessel. In some aspects, the first reactor vessel can comprise a first microbial inoculant composition. In some aspects, the methods can further comprise collecting biogas from the MBBR. In some aspects, the biogas collected from the MBBR can be methane and/or CO₂ or a mixture thereof. In some aspects, the biogas collected from the MBBR can be methane, hydrogen, ammonia, CO₂, hydrogen sulfide, N₂O or any combination thereof.

In some aspects, the methods can further comprise the step of transferring a portion of the digested or partially digested landfill leachate from the first or second reactor vessel to a third reactor vessel and contacting the digested or partially digested landfill leachate in the third reactor vessel with a third microbial inoculant composition to facilitate the digestion of the landfill leachate under conditions to convert ammonia into nitrates. In some aspects, the methods can further comprise collecting liquids from the third reactor vessel. In some aspects, the method can be continuous. In some aspects, biomass can be continuously added or sporadically added to the first reactor vessel.

In some aspects, the first microbial inoculant can comprise a first bacterial strain and a second bacterial strain, wherein the first bacterial strain comprises Clostridium spp. In some aspects, the 16S sequence of Clostridium spp. can comprise any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2.

In some aspects, the first microbial inoculant composition in step a) can decrease or suppress one or more methanogens in the first reactor vessel. In some aspects, the first microbial inoculant composition can create or change the environment in the first reactor vessel such than the metabolism of any of methanogens present in the first reactor vessel is suppressed.

In some aspects, the second microbial inoculant composition can be different than the first microbial inoculant composition. In some aspects, the second microbial inoculant composition can comprise one or more methanogen producers. Examples of methanogoen producers include but are not limited to Methanobacterium bryantii, Methanobacterium formicum, Methanobrevibacter arboriphilicus, Methanobrevibacter gottschalkii, Methanobrevibacter ruminantium, Methanobrevibacter smithii, Methanococcus chunghsingensis, Methanococcus burtonii, Methanococcus aeolicus, Methanococcus deltae, Methanococcus jannaschii, Methanococcus maripaludis, Methanococcus vannielii, Methanocorpusculum labreanum, Methanoculleus bourgensis (Methanogenium olentangyi and Methanogenium bourgense), Methanoculleus marisnigri, Methanoflorens stordalenmirensis, Methanofollis liminatans, Methanogenium cariaci, Methanogenium frigidum, Methanogenium organophilum, Methanogenium wolfei, Methanomicrobium mobile, Methanopyrus kandleri, Methanoregula boonei, Methanosaeta concilii, Methanosaeta thermophile, Methanosarcina acetivorans, Methanosarcina barkeri, Methanosarcina mazei, Methanosphaera stadtmanae, Methanospirillium hungatei, Methanothermobacter defluvii (Methanobacterium defluvii), Methanothermobacter thermautotrophicus (Methanobacterium thermoautotrophicum), Methanothermobacter thermoflexus (Methanobacterium thermoflexum), Methanothermobacter wolfei (Methanobacterium wolfei), and Methanothrix sochngenii.

In some aspects, the third microbial inoculant composition can comprise a first bacterial strain and a second bacterial strain. In some aspects, the first bacterial strain can comprise Clostridium spp. In some aspects, the 16S sequence of Clostridium spp. can comprise any one of the Clostridium spp. listed in Table 1 or Table 2. In some aspects, the second bacterial strain can comprise an aquatic Pseudomonas spp. bacteria with a 16S nucleic acid sequence that is at least about 97% identical to any one of the Pseudomonas spp. bacteria listed in Table 1 or Table 2. In some aspects, the third microbial inoculant composition can be the same as the first microbial inoculant composition in step a).

In some aspects, the landfill leachate can comprise feedstock, a plant material, an animal material, food, water, industrial waste or organic waste products, residual waste thereof, or combination thereof. In some aspects, the landfill leachate can be pretreated. In some aspects, the landfill leachate can be pretreated to enhance digestibility. Examples of pretreatment approaches include but are not limited to chemical (e.g., alkaline, acidic and inorganic salts), physical (e.g., microwaves and liquid hot water), and biological (e.g., enzymatic and fungal). In some aspects, the landfill leachate can pretreated with a microbial inoculant composition. In some aspects, the microbial inoculant composition can comprise a first bacterial strain and a second bacterial strain. In some aspects, the first bacterial strain can comprise Clostridium spp. In some aspects, the 16S sequence of Clostridium spp. can comprise any one of the Clostridium spp. listed in Table 1 or Table 2. In some aspects, the second bacterial strain can comprise an aquatic Pseudomonas spp. bacteria with a 16S nucleic acid sequence that is at least about 97% identical to any one of the Pseudomonas spp. bacteria listed in Table 1 or Table 2. In some aspects, the first microbial inoculant can comprise one or more of the microbes listed in Table 1, Table 2 or Table 3. In some aspects, the first microbial inoculant can comprise at least one different microbial strain, wherein the 16S sequence of the one different microbial strain can comprise a 16S sequence that is at least about 97% identical to one or more of the 16S sequences listed in Table 1, Table 2 or Table 3.

In some aspects, the landfill leachate and the first microbial inoculant composition can be introduced into the first reactor vessel at the same time. In some aspects, the landfill leachate can be introduced into the first reactor vessel before or after the first microbial inoculant composition is introduced into the first reactor vessel. In some aspects, the landfill leachate and second microbial inoculant composition can introduced into the second reactor vessel at the same time. In some aspects, the landfill leachate can be introduced into the second reactor vessel before or after the second microbial inoculant composition is introduced into the second reactor vessel.

In some aspects, the MBBR can comprise a microbial inoculant composition. In some aspects, the microbial inoculant composition can be the same microbial inoculant composition present in the second reactor vessel.

In some aspects, the method can comprise maintaining the MBBR at an ORP that can be similar or the same ORP as the second ORP in the second reactor vessel. In some aspects, the ORP can be maintained in step a) between around −50 mV and −600 mV. In some aspects, the ORP can be maintained in step c) between around −100 mV and less than 1000 mV. In some aspects, the ORP can be maintained in step c) between around −300 mV and less than −400 mV. In some aspects, ORP can be be maintained at a negative ORP. In some aspects, the ORP of the first reactor vessel can be less than −50 mV. In some aspects, the ORP of the second reactor vessel can be between less than 0 and greater than −150 mV. In some aspects, the method can further comprise maintaining the ORP in the third reactor vessel between around −80 mV and −800 mV.—In some aspects, the ORP of the third reactor vessel can be zero or below.

In some aspects, the first reactor vessel can be maintained with a low oxygen level. In some aspect, the oxygen level in the first reactor vessel can be less than 2 ppM.

In some aspects, the second reactor vessel can be maintained with an oxygen level higher than the first reactor vessel. In some aspect, the oxygen level in the second reactor vessel can be greater than 2 ppM.

In some aspects, the third reactor vessel can be maintained with a low oxygen level. In some aspect, the oxygen level in the third reactor vessel can be less than 2 ppM.

In some aspects, the MBBR can be maintained with an oxygen level higher than the first reactor vessel. In some aspect, the oxygen level in the MBBR can be greater than 2 ppM. In some aspects, the MBBR can have an ORP greater than −150 mV.

In some aspects, the oxygen-containing gas can be pure oxygen, a blend of oxygen and inert gas(es) or an oxygen liberating source (e.g., hydrogen peroxide).

In some aspects, the methods can further comprise maintaining a pH level of the contents of the first reactor vessel at a first pH level or within a first pH range. In some aspects, the first pH level can be less than 6 or the first pH range can be between 1 and 6. In some aspects, the pH of the first reactor vessel can be less than 5. In some aspects, the pH of the first reactor vessel can be 4, 3, 2 or 1. In some aspects, the pH of the second reactor vessel can be between 1 and 8. In some aspects, the pH of the second reactor vessel can be between 2 and 6. In some aspects, the first pH level of the third reactor vessel can be less than 6 or the first pH range can be between 1 and 6. In some aspects, the pH of the third reactor vessel can be less than 5. In some aspects, the pH of the third reactor vessel can be 4, 3, 2 or 1.

In some aspects, the methods can further comprise maintaining the temperature of the contents of the first reactor vessel at above 0° C. In some aspects, the temperature of the first reactor vessel can be maintained between 65° F. and 135° F. In some aspects, the methods can further comprise maintaining the temperature of the contents of the second reactor vessel at above 0° C. In some aspects, the temperature of the second reactor vessel can be maintained between 65° F. and 135° F. In some aspects, the methods can further comprise maintaining the temperature of the contents of the third reactor vessel at above 0° C. In some aspects, the temperature of the third reactor vessel can be maintained between 65° F. and 135° F.

In some aspects, the landfill leachate in the third reactor vessel can be separated into a solid portion and a liquid portion.

In some aspects, the methods can further comprise removing a portion of water from the solid portion. In some aspects, the step of removing a portion of water from the solid portion can be via evaporation, centrifugation, flocculation, filtration, settling, or the like. In some aspects, the step of removing a portion of water from the solid portion can be used to produce a concentrate of carbon, one or more metals, fertilizers or other components from the solubilized and concentrated materials from the solid portion.

In some aspects, the portion of water removed from the solid portion can be nitrate enriched. As such, in some aspects, the methods disclosed herein can further comprise collecting nitrate enriched water from the biomass or solid portions disclosed herein. In some aspects, the nitrate can be soluable and not volatile, and can be concentrated to be returned to agriculture or industry.

In some aspects, the solid portion of the landfill leachate can be separated into primitive carbon(s). In some aspects, the liquid portion comprises inorganic plant nutrients. In some aspects, the total amounts of inorganic plant nutrients in the liquid portion can be increased. In some aspects, the overall amount of the solid portion can be decreased. In some aspects, the methods can further comprise collecting a portion of the liquid portion from the third reactor vessel. In some aspects, the liquid portion can be used as a fertilizer. In some aspects, the liquid portion can be used as a biomass. In some aspects, the methods can further comprise collecting a portion of the solid portion from the third reactor vessel. In some aspects, the solid portion collected can be reused on the field as a compost source. In some aspects, the solid portion can be used as a fertilizer. In some aspects, the solid portion can be used as a biomass.

In some aspects, the amount of acetate that can be reduced in the liquid fraction can be at least 5% relative prior to the digesting step. In some aspects, the amount of the acetate that is reduced is between 5% and 99% relative prior to the digesting step. In some aspects, the amount of the acetate that is reduced is at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or any percent decrease in between relative prior to the digesting step.

Also disclosed herein are methods of producing hydrogen in a landfill leachate. In some aspects, the landfill leachate can serve as a reactor vessel. In some aspects, the method can comprise applying a composition comprising two or more bacterial strains to the landfill leachate. In some aspects, a first bacterial strain can comprise Clostridium spp. In some aspects, the 16S sequence of Clostridium spp. can comprise any one of the Clostridium spp. listed in Table 1 or Table 2. In some aspects, a second bacterial strain can comprise an aquatic Pseudomonas spp. bacteria with a 16S nucleic acid sequence that is at least about 97% identical to any one of the Pseudomonas spp. bacteria listed in Table 1 or Table 2. In some aspects, the methods can comprise collecting samples from the landfill leachate. In some aspects, the methods can include applying a microbial inoculant composition to landfills. The application of the microbial inoculant composition to landfills can be by any suitable method. In some aspects, the application of the microbial inoculant composition to landfills can be in the form of a liquid or a spray. In some aspects, the method can comprise contacting the landfill leachate with the microbial inoculant composition under anaerobic conditions to facilitate the digestion of the landfill leachate. In some aspects, the digestion of the landfill leachate can be full or partial. In some aspects, the methods can comprise collecting hydrogen gas from the landfill leachate and transferring a portion of the digested or partially digested landfill leachate to a second reactor vessel.

In some aspects, the any of the microbial inoculant compositions described herein can be applied such that there exists 10² to 10¹², 10³ to 10¹², 10⁴ to 10¹², 10⁵ to 10¹², 10⁶ to 10¹², 10⁷ to 10¹², 10⁸ to 10¹², 10⁹ to 10¹², 10¹⁰ to 10¹², 10¹¹ to 10¹², 10² to 10¹¹, 10³ to 10¹¹, 10⁴ to 10¹¹, 10⁵ to 10¹¹, 10⁶ to 10¹¹, 10⁷ to 10¹¹, 10⁸ to 10¹¹, 10⁹ to 10¹¹, 10¹⁰ to 10¹¹, 10² to 10¹⁰, 10³ to 10¹⁰, 10⁴ to 10¹⁰, 10⁵ to 10¹⁰, 10⁶ to 10¹⁰, 10⁷ to 10¹⁰, 10⁸ to 10¹⁰, 10⁹ to 10¹⁰, 10² to 10⁹, 10³ to 10⁹, 10⁴ to 10⁹, 10⁵ to 10⁹, 10⁶ to 10⁹, 10⁷ to 10⁹, 10⁸ to 10⁹, 10² to 10⁸, 10² to 10⁸, 10⁴ to 10⁸, 10⁵ to 10⁸, 10⁶ to 10⁸, 10⁷ to 10⁸, 10² to 10⁷, 10³ to 10⁵, 10⁴ to 10⁵, 10² to 10⁴, 10³ to 10⁴, 10² to 10³, 10¹², 10¹¹, 10¹⁰, 10⁹, 10⁸, 10⁷, 10⁶, 10⁵, 10⁴, 10³, or 10² total microbial cells per gram or milliliter of the composition.

In some aspects, the biomass can be uniformly coated with one or more layers of the microbes and/or microbial compositions disclosed herein, using conventional methods of mixing, spraying, or a combination thereof through the use of treatment application equipment that is specifically designed and manufactured to accurately, safely, and efficiently apply coatings. Such equipment uses various types of coating technology such as rotary coaters, drum coaters, fluidized bed techniques, spouted beds, rotary mists, or a combination thereof. Liquid treatments such as those of the present disclosure can be applied via either a spinning “atomizer” disk or a spray nozzle, which evenly distributes the microbial composition onto the feed as it moves though the spray pattern. In some aspects, the feed can then be mixed or tumbled for an additional period of time to achieve additional treatment distribution and drying.

In some aspects, the feed coats of the present disclosure can be up to 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780 μm, 790 μm, 800 μm, 810 μm, 820 μm, 830 μm, 840 μm, 850 μm, 860 μm, 870 μm, 880 μm, 890 μm, 900 μm, 910 μm, 920 μm, 930 μm, 940 μm, 950 μm, 960 μm, 970 μm, 980 μm, 990 μm, 1000 μm, 1010 μm, 1020 μm, 1030 μm, 1040 μm, 1050 μm, 1060 μm, 1070 μm, 1080 μm, 1090 μm, 1100 μm, 1110 μm, 1120 μm, 1130 μm, 1140 μm, 1150 μm, 1160 μm, 1170 μm, 1180 μm, 1190 μm, 1200 μm, 1210 μm, 1220 μm, 1230 μm, 1240 μm, 1250 μm, 1260 μm, 1270 μm, 1280 μm, 1290 μm, 1300 μm, 1310 μm, 1320 μm, 1330 μm, 1340 μm, 1350 μm, 1360 μm, 1370 μm, 1380 μm, 1390 μm, 1400 μm, 1410 μm, 1420 μm, 1430 μm, 1440 μm, 1450 μm, 1460 μm, 1470 μm, 1480 μm, 1490 μm, 1500 μm, 1510 μm, 1520 μm, 1530 μm, 1540 μm, 1550 μm, 1560 μm, 1570 μm, 1580 μm, 1590 μm, 1600 μm, 1610 μm, 1620 μm, 1630 μm, 1640 μm, 1650 μm, 1660 μm, 1670 μm, 1680 μm, 1690 μm, 1700 μm, 1710 μm, 1720 μm, 1730 μm, 1740 μm, 1750 μm, 1760 μm, 1770 μm, 1780 μm, 1790 μm, 1800 μm, 1810 μm, 1820 μm, 1830μ, 1840 μm, 1850 μm, 1860μ, 1870 μm, 1880μ, 1890 μm, 1900 μm, 1910 μm, 1920 μm, 1930 μm, 1940 μm, 1950 μm, 1960 μm, 1970 μm, 1980 μm, 1990 μm, 2000 μm, 2010 μm, 2020 μm, 2030 μm, 2040 μm, 2050 μm, 2060 μm, 2070 μm, 2080 μm, 2090 μm, 2100 μm, 2110 μm, 2120 μm, 2130 μm, 2140 μm, 2150 μm, 2160 μm, 2170 μm, 2180 μm, 2190 μm, 2200 μm, 2210 μm, 2220 μm, 2230 μm, 2240 μm, 2250 μm, 2260 μm, 2270 μm, 2280 μm, 2290 μm, 2300 μm, 2310 μm, 2320 μm, 2330 μm, 2340 μm, 2350 μm, 2360 μm, 2370 μm, 2380 μm, 2390 μm, 2400 μm, 2410 μm, 2420 μm, 2430 μm, 2440 μm, 2450 μm, 2460 μm, 2470 μm, 2480 μm, 2490 μm, 2500 μm, 2510 μm, 2520 μm, 2530 μm, 2540 μm, 2550 μm, 2560 μm, 2570 μm, 2580 μm, 2590 μm, 2600 μm, 2610 μm, 2620μ, 2630 μm, 2640 μm, 2650 μm, 2660 μm, 2670 μm, 2680 μm, 2690μ, 2700 μm, 2710 μm, 2720 μm, 2730 μm, 2740 μm, 2750 μm, 2760 μm, 2770 μm, 2780 μm, 2790 μm, 2800 μm, 2810 μm, 2820 μm, 2830 μm, 2840 μm, 2850 μm, 2860 μm, 2870 μm, 2880 μm, 2890 μm, 2900 μm, 2910 μm, 2920 μm, 2930 μm, 2940 μm, 2950 μm, 2960 μm, 2970 μm, 2980 μm, 2990 μm, or 3000 μm thick.

In some aspects, the microbial cells can be coated freely onto any number of compositions or they can be formulated in a liquid or solid composition before being coated onto a composition. For example, a solid composition comprising the microorganisms can be prepared by mixing a solid carrier with a suspension of the spores until the solid carriers are impregnated with the spore or cell suspension. This mixture can then be dried to obtain the desired particles.

In some other aspects, it is contemplated that the solid or liquid compositions of the present disclosure further contain functional agents e.g., activated carbon, minerals, vitamins, and other agents capable of improving the quality of the products or a combination thereof.

Methods of coating and compositions in use of said methods that are known in the art can be particularly useful when they are modified by the addition of one of the embodiments of the present disclosure. Such coating methods and apparatus for their application are disclosed in, for example: U.S. Pat. Nos. 8,097,245 and 7,998,502; and PCT Pat. App. Pub. Nos. WO 2008/076975, WO 2010/138522, WO 2011/094469, WO 2010/111347, and WO 2010/111565 each of which is incorporated by reference herein.

In some aspects, the microbes or microbial compositions of the present disclosure can exhibit a synergistic effect, on one or more of the traits, in the presence of one or more of the microbes or microbial compositions coming into contact with one another.

The microbial inoculant compositions disclosed here can be applied to a biomass by any suitable method. As described above, the microbial inoculant composition may be formulated with a biocompatible adhesive agent that allows the microbial inoculant composition to be applied to, and adhere to, a biomass.

In some aspects, the methods can include applying the microbial inoculant composition to landfills. The application of the microbial inoculant composition to landfills can be by any suitable method. In some aspects, the application of the microbial inoculant composition to landfills can be in the form of a liquid or a spray.

In some aspects, a formulation of the microbial inoculant composition can comprise a predetermined moisture content. In some aspects, the minimum moisture content can be at least 5% such as, for example, at least 6%, at least 7%, at least 8%, at least 9%, at least 10%, at least 15%, at least 20%, at least 25%, at least 30%, or at least 50%.

In some aspects, a formulation of the microbial inoculant composition can comprise a sugar (e.g., cane sugar or sucrose) and vinegar (e.g., white vinegar). The sugar can provide a metabolic carbon source. The vinegar can provide an acidic pH and/or an alternative carbon source. As an alternative to, or in addition to, the use of vinegar to regulate pH, the microbial inoculant composition can comprise Lactobacillus plantarum, as described herein, to help maintain an acidic pH once the microbial inoculant composition is applied to the biomass.

In some aspects, a formulation of the microbial inoculant composition can comprise lactic acid media to provide an acidic pH.

In some aspects, a formulation of the microbial inoculant composition can comprise glycerol as a dispersion medium.

Hydrogen can be selectively produced following exposure of a first microbial inoculant composition to a severe dried condition, a dried, semi-dried, or low moisture condition. In some aspects, the gas content of the first reactor vessel can include hydrogen and carbon dioxide. The percentage of hydrogen in the outflow gas can range from about 15% to about 80%, or from about 20% to about 55%. Most of the remaining gas (from about 85% to about 20%, or 80% to 55% about 45%, respectively) can be comprised of carbon dioxide. It is understood that the ratio of hydrogen to carbon dioxide can vary with time (reaction time) during the contacting step. In some aspects, methane gas is not detected during hydrogen production or in the first reactor vessel. In some aspects, the first reactor vessel can be in an aqueous state. In some aspects, at least 15% water can be present in the first reactor vessel. In some aspects, the amount of water present in the first reactor vessel can be sufficient to make the solution flowable.

Disclosed herein are methods of producing methane. In some aspects, the methane producing step (e.g., methanogenesis), methanogenic bacteria such as hydrogen-utilizing bacteria and acetate utilizing bacteria consume hydrogen and acetate to produce methane. Methane gas can be produced under anaerobic conditions when the mixed anaerobic bacterial community is comprised of active methanogenic bacteria.

For methane production, the methane content in the second reactor vessel can range from about 60% to about 85%, from about 65% to about 80%, from about 70% to about 75% methane. In some aspects, the methane content in the reactor vessel can be about 65% to about 80%. Other gas components in the second reactor vessel may include carbon dioxide (e.g., 40%, 35%, 30%, 25%, 20%, 15%, 10% or less carbon dioxide), hydrogen sulfide (in trace amounts), and water vapor as air (in trace amounts). In some aspects, hydrogen gas is not detected in the outflow gas when selectively producing methane.

In some aspects, biomass or biomass feedstock pretreatments can include, but are not limited to, ammonia fiber explosion (AFEX), steam explosion, comminution, fungal pretreatment, electrical pretreatment, acid pretreatment, alkaline pretreatment, sulfur dioxide treatment, and radiation pretreatment.

The pH level of the contents within the any of reactor vessels described herein can also be controlled. In the hydrogen production step, pH level of contents of the reactor vessel can be set at a first pH level or within a first pH range. The first pH level can be a neutral pH or within a neutral pH range. The pH level can be adjusted to a neutral pH by, for example, the addition of HCl, NaOH, sodium biocarbonate, KOH, NH₄OH, lime, or calcium carbonate. In some aspects, the biomass feedstock can introduced into the first reactor vessel and the pH level can be set at neutral pH for liquefaction and hydrolysis of the feedstock. During the production of hydrogen, the pH level of the contents within the first reactor vessel drops to about 1 to about 6.0. In some aspects, a drop in pH in the first reactor vessel can indicate a positive hydrogenic reaction.

In some aspects, after the collection of hydrogen gas, the pH level can be set to a second pH level or within a second pH range. In some aspects, after the collection of hydrogen, the pH level can be increased to a second pH level or within a second pH range for the production of methane gas. It is also contemplated that rehydration (or dilution) of the biomass feedstock in the second reactor vessel (to selectively produce methane) can result in an increase in pH level. In some aspects, an increase in pH following a hydrogen-producing acidogenesis reaction, activates non-acidogenic bacteria (e.g., methanogenic bacteria) which leads to the production of methane.

In some aspects, the pH of the contents within any of the reactor vessels disclosed herein can be set at a range at the beginning of an anaerobic digestion reaction for the production of hydrogen and/or methane or the pH may be monitored and maintained during the anaerobic reaction producing hydrogen and/or methane.

In some aspects, the pH range can be from about pH 4.5 to about pH 7.5, from about pH 5.0 to about pH 7.0, from about pH 5.5 to about pH 6.5, pH 1 to about pH 3, pH 3 to about pH 5, or from about pH 5.5 to about pH 6.0. In some aspects, the pH of the first reactor vessel can be less than 6 or be between 1 and 6. In some aspects, the pH of the first reactor vessel can be less than 5. In some aspects, the pH of the first reactor vessel can be less than 6.8 or be between 2 and 6.8. In some aspects, the pH of the first reactor vessel can be 6.8, 6.7, 6.6, 6.5, 6.4, 6.3, 6.2, 6.1, 6, 5, 4, 3, 2 or 1. In some aspects, the pH of the second reactor vessel can be between 1 and 8. In some aspects, the pH of the second reactor vessel can be between 2 and 6. In some aspects, the pH of the second reactor vessel can be between 2 and 9.5. In some aspects, the first pH level of the third reactor vessel can be less than 6.8 or the first pH range can be between 2 and 6.8. In some aspects, the first pH level of the third reactor vessel can be less than 6 or the first pH range can be between 1 and 6. In some aspects, the pH of the third reactor vessel can be less than 5. In some aspects, the pH of the third reactor vessel can be 6.8, 6.7, 6.6, 6.5, 6.4, 6.3, 6.2, 6.1, 6, 5, 4, 3, 2 or 1. In some aspects, the pH level of the third reactor vessel can be less than 6.8 or the first pH range can be between 2 and 6.8 to initiate hydrogen production.

In some aspects, the pH to produce hydrogen or methane under anaerobic conditions can differ depending on the source of the biomass feedstock. The pH level of the contents within any of the reactor vessels can be controlled by maintaining natural buffering capacity, adding buffering chemicals, or by using a pH controller (e.g., a standard electric pH monitor probe or pH meter). The pH level of the contents within the reactor vessel can be monitored continuously or periodically. Adjustments in pH can be made by automatic addition of acid or base when the pH reaches a preset threshold to avoid a drop or increase in pH. In some aspects, adjustments in pH can be made by periodic monitoring and subsequent addition of acid or base. In some aspects, the ORP can be maintained directly or indirectly.

In some aspects, the ORP can be changed and/or maintained in the first bioreactor by changing one or more of the following: the pH, oxygen levels, temperature and the presence of facultative aerobes in the bioreactor. In some aspects, a negative ORP refers to an ORP that is less than 0. In some aspects, the negative ORP can be less than −50 mV. In some aspects, the ORP can be less than −50 mV to produce, for example, hydrogen. In some aspects, a negative ORP can refer to a mV of about 50 mV or lower indicating a microbially active system in active reduction of one or more carbonaceous materials. In some aspects, the oxygen level in the first bioreactor can be such that an anaerobic condition is achieved that is substantially free of dissolved oxygen and promotes anaerobic conditions. In some aspects, the oxygen level in the first or second bioreactor can be less than 2 ppM. In some aspects, the pH can be less than 5.

In some aspects, the pH can be between 1 and 2. In some aspects, the pH can be between 1 and 3. In some aspects, the pH can be between 1 and 4. In some aspects, the pH can be between 1 and 5. In some aspects, the pH can be about 1, about 2, about 3, about 4 or about 5. In some aspects, the temperature can be at least above 0° C. In some aspects, the temperature can be between about 40° F.-240° F. In some aspects, the temperature can be between about 65° F.-135° F. In some aspects, facultative aerobes can be included in the microbial inoculant compositions to reduce ORP, and further, so that anaerobes can additionally reduce ORP.

In some aspects, the ORP can be changed and/or maintained in the second bioreactor by changing one or more of the following: the pH, oxygen levels, temperature and the presence of facultative aerobes in the bioreactor. In some aspects, a negative ORP refers to an ORP that is less than 0. In some aspects, the ORP can be greater than −50 mV. In some aspects, the negative ORP can be greater than −150 mV. In some aspects, the ORP can be less than 0 mV. In some aspects, the oxygen level in the first or second bioreactor can be less than 2 ppM. In some aspects, the oxygen level of the second bioreactor can be at or greater than 2 mg/L. In some aspects, the pH can be less than 5. In some aspects, the pH can be between 1 and 2. In some aspects, the pH can be between 1 and 3. In some aspects, the pH can be between 1 and 4. In some aspects, the pH can be between 1 and 5. In some aspects, the pH can be between 1 and 6. In some aspects, the pH can be between 1 and 7. In some aspects, the pH can be between 1 and 8. In some aspects, the pH can be between 2 and 6. In some aspects, the pH can be about 1, about 2, about 3, about 4 or about 5. In some aspects, the temperature can be at least above 0° C. In some aspects, the temperature can be between about 40° F.-240° F. In some aspects, the temperature can be between about 65° F.-135° F. In some aspects, facultative aerobes can be included in the microbial inoculant compositions to reduce ORP, and further, so that anaerobes can additionally reduce ORP.

In some aspects, the ORP can be changed and/or maintained in the third bioreactor by changing one or more of the following: the pH, oxygen levels, temperature and the presence of facultative aerobes in the bioreactor. In some aspects, a negative ORP refers to an ORP that is less than 0. In some aspects, the negative ORP can be greater than −150 mV. In some aspects, the ORP can be less than 0 mV. In some aspects, a negative ORP can refer to a mV of about 50 mV or lower indicating a microbially active system in active reduction of one or more carbonaceous materials. In some aspects, the oxygen level in the first bioreactor can be such that an anaerobic condition is achieved that is substantially free of dissolved oxygen and promotes anaerobic conditions. In some aspects, the oxygen level in the first or third bioreactor can be less than 2 ppM. In some aspects, the pH can be less than 5. In some aspects, the pH can be between 1 and 2. In some aspects, the pH can be between 1 and 3. In some aspects, the pH can be between 1 and 4. In some aspects, the pH can be between 1 and 5. In some aspects, the pH can be between 1 and 6. In some aspects, the pH can be between 1 and 7. In some aspects, the pH can be between 1 and 8. In some aspects, the pH can be between 2 and 6. In some aspects, the pH can be about 1, about 2, about 3, about 4 or about 5. In some aspects, the temperature can be at least above 0° C. In some aspects, the temperature can be between about 40° F.-240° F. In some aspects, the temperature can be between about 65° F.-135° F. In some aspects, facultative aerobes can be included in the microbial inoculant compositions to reduce ORP, and further, so that anaerobes can additionally reduce ORP. In some aspects, a pump can be used to push air in to bring the ORP to greater than −150 mV.

In some aspects, the first bioreactor disclosed herein can comprise media. In some aspects, the media does not include or includes only trace amounts of any antibiotic or any pathogen to any of the organisms or microbes present in the first bioreactor. In some aspects, the media can have water added or can contain water. In some aspects, the organisms or microbes present in the first bioreactor can be placed on a solid (e.g., a solid biomass) prior to coming into contact with the media or the water added or the slurry that occurs after the water and solids are introduced together. In some aspects, the media can be pretreated to remove any unwanted substances (e.g., antibiotics or pathogens). In some aspects, the biomass can be pretreated before coming into contact with the media or being placed in the first bioreactor. For example, the biomass can be pretreated with UV to remove copper sulfate that may be present (from exposure to the feet of cows that stepped into a footbath containing copper sulfate to kill pathogens prior to feeding). In some aspects, the media can comprise a carbon source, a nitrogen source, phosphorus, iron (e.g., iron in iron+3 form including, but not limited to magnetite), or a combination thereof. In some aspects, the media can further comprise one or more micronutrients, one or more vitamins, or a combination thereof. In some aspects, the media can be any feedstock that can be added to an aqueous system capable of supporting microbial life. In some aspects, the media can be supplemented with one or more buffers, one or more acidifiers, or a combination thereof. In some aspects, the media can be supplemented with one or more buffers including but not limited to carbonates and bicarbonates. In In some aspects the one or more buffers can be added to the media to reduce the pH. In some aspects, the media can be supplemented with one or more acidifiers including but not limited to sulfuric acid, phosphoric acid or other acids. In some aspects, the one or more acidifiers can be added to the media to reduce the pH.

In some aspects, the second bioreactor disclosed herein can comprise media. In some aspects, the media does not include or includes only trace amounts of any antibiotic or any pathogen to any of the organisms or microbes present in the second bioreactor. In some aspects, the media can be pretreated to remove any unwanted substances (e.g., antibiotics or pathogens). In some aspects, the biomass can be pretreated before coming into contact with the media or being placed in the first bioreactor. For example, the biomass can be pretreated with UV to remove copper sulfate that may be present (from exposure to the feet of cows that stepped into a footbath containing copper sulfate to kill pathogens prior to feeding). In some aspects, the media can comprise a carbon source, a nitrogen source, phosphorus, iron (e.g., iron in iron+3 form including, but not limited to magnetite), or a combination thereof. In some aspects, the media can further comprise one or more micronutrients, one or more vitamins, or a combination thereof. In some aspects, the media can be any feedstock that can be added to an aqueous system capable of supporting microbial life. In some aspects, the media can be supplemented with one or more buffers, one or more acidifiers, or a combination thereof. In some aspects, the media can be supplemented with one or more buffers including but not limited to carbonates and bicarbonates. In In some aspects the one or more buffers can be added to the media to reduce the pH. In some aspects, the media can be supplemented with one or more acidifiers including but not limited to sulfuric acid, phosphoric acid or other acids. In some aspects, the one or more acidifiers can be added to the media to reduce the pH.

In some aspects, the third bioreactor disclosed herein can comprise media. In some aspects, the media does not include or includes only trace amounts of any antibiotic or any pathogen to any of the organisms or microbes present in the third bioreactor. In some aspects, the media can be pretreated to remove any unwanted substances (e.g., antibiotics or pathogens). In some aspects, the biomass can be pretreated before coming into contact with the media or being placed in the first bioreactor. For example, the biomass can be pretreated with UV to remove copper sulfate that may be present (from exposure to the feet of cows that stepped into a footbath containing copper sulfate to kill pathogens prior to feeding). In some aspects, the media can comprise a carbon source, a nitrogen source, phosphorus, iron (e.g., iron in iron+3 form including, but not limited to magnetite), or a combination thereof. In some aspects, the media can further comprise one or more micronutrients, one or more vitamins, or a combination thereof. In some aspects, the media can be any feedstock that can be added to an aqueous system capable of supporting microbial life. In some aspects, the media can be supplemented with one or more buffers, one or more acidifiers, or a combination thereof. In some aspects, the media can be supplemented with one or more buffers including but not limited to carbonates and bicarbonates. In In some aspects the one or more buffers can be added to the media to reduce the pH. In some aspects, the media can be supplemented with one or more acidifiers including but not limited to sulfuric acid, phosphoric acid or other acids. In some aspects, the one or more acidifiers can be added to the media to reduce the pH.

Reaction time (e.g., the duration of the time beginning from introducing the biomas into the first reactor vessel (or the second reactor vessel) to the completion of the hydrogenic phase for hydrogen production (or the methanogenic phase for methane production)), can vary to produce hydrogen or methane. In some aspects, the reaction time can continue until a pH of about 1.8 is reached or the carbon is sufficiently converted to organic acids that cannot be consumed.

In some aspects, reaction or residence time can be the time during which the biomass and the microbial inoculant are in contact and producing biogas. For hydrogen production, the reaction or residence time can be from about 1 day to about 90 days, about 1 day to about 80 days, about 1 day to about 70 days, about 1 day to about 60 days, about 1 day to about 50 days, about 1 day to about 40 days, about 1 to about 30 days, or about 1 day to about 20 days. The reaction or residence time can be prolonged or shortened depending on the hydrogen-producing characteristics (for example, different biomass feedstocks). In some aspects, the reaction or residence time to favor methane production can be about 5 to about 40 days, about 10 to about 40 days, or about 20 to about 40 days. In some aspects, hydrogen can be produced using the methods disclosed herein in about 1.5 hours after inoculation in the first reactor vessel and it can be continued to be produced until a pH of about 1.8 is reached or the carbons are sufficiently converted to organic acids.

The hydrogen and methane gas produced by the methods disclosed herein using the microbial inoculants also disclosed herein can be collected using any technique known to one of ordinary skill in the art. In some aspects, when a continuous reactor is used, hydrogen and methane gas can be collected using a plastic device or gas bag (e.g., Tedlar gas bag). In some aspects, a separate gas bag can be attached to the reactor vessel to maintain constant pressure or a spring-laded check valve to relieve pressure can be employed. In some aspects, gas collection and storage methods can differ depending on the purpose or utilization for the gas. In some aspects, collection covers can be used. In some aspects, volumetric gas meters, wet tip gas meters, or gas chromatographs can be used to measure the gas production.

In some aspects, biogas can be collected and measured from a batch reactor sealed with a butyl rubber stopper and an aluminum crimp using wetted syringes.

In some aspects, any of the gases, biogases, liquid portions, solid portions or byproducts produced in any of the bioreactors including the landfill leachate can be uses a source or component in any of the other bioreactors disclosed herein.

### EXAMPLES

### Example 1. Reactor Systems for Selectively and Separately Producing Hydrogen and Methane

FIG. 1 shows an exemplary reactor system that can be used in the methods disclosed herein. FIG. 2 shows the chemistry of the anaerobic digestion. FIG. 3 is a schematic showing the stream flowing through a moving biofilm bed reactor (MBBR) from hydrogen forming to methane forming and then back to hydrogen forming when it re-enters the hydrogen reactor

Hydrogen production using a first reactor vessel was performed using pure cane sugars in an aqueous solution that generated H₂ in excess of 10,000 ppm for a period of between 72 and 120 hours with a maximum flow of 1.20 ml/s in a stainless steel 2,000 gallon vessel using 100 lbs of sugars. Temperatures used were 80° F. to 109° F. The greatest volume of gas created was at 109° F. Hydrogen was initiated when the ORP dropped to 50 mV from a 200 mV initiating state. This was a non-mixed system and the lower levels had become anaerobic and had an ORP of 0 mV and began the hydrogen production in the lower levels rapidly converting the first reactor vessel from a pH of 7.5 to a pH of 6.8 prior to imitating hydrogen production.

### Example 2. Thin Stillage from a Sugar Beet Facility

Feedstock: 1. Water from processing with a little sugar left in it; and/or
   2. Bagasse. The left-over solids from the processing of the sugar.
Process Steps:
   I. Hydrogen
   II. Methane
   III. Hydrogen

Methodology: The microbial inoculant (a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2) is added to an aqueous solution containing one or more of the feedstocks listed above and the solution is added to the first reactor vessel. The pH and ORP are allowed to come into range through biological activity utilizing a sealed oxygen impermeable reactor vessel. For example, pH of 6 to 3 for hydrogen production and a pH of 4 to 8.5 for methane production. ORP is reduced by 50 mV from the starting point for hydrogen and will go down as low as −600 mV or more depending on the amount of iron in the feedstock, and above −50 mV for methane production. The ORP for hydrogen production to start is based on a fall of 50 points from starting point, and is not based on a fixed starting point. This allows the hydrogen to be produced and is captured through a piping system attached to the top of the first reactor vessel. The reaction is allowed to continue until all or most the feedstock organic material is converted to organic acids that will lower the pH to the point that the reaction is impaired. All or a portion of the aqueous solution is transferred to a second reactor vessel. This solution is aerated with a gas containing oxygen to raise the dissolved oxygen above 2 mg/L and this oxygenated state of the aqueous solution is maintained until the pH and ORP rises to the desired levels (greater than 4 for pH and greater than −50 mV for ORP preferred in this example). Methane is produced while maintaining a dissolved oxygen greater than 2 mg/L by injecting an oxygenated gas into the second reactor vessel. Then the aqueous solution after methane production slows to a predetermined level that can be either transferred back to the first reactor vessel or to the third reactor vessel for additional hydrogen creation. This will conserve total water volume and usage in the system. The third reactor vessel is allowed to become anoxic (dissolved oxygen lower than 2 mg/L) and the hydrogen microbes will resume hydrogen production from the remaining organic carbon feedstock. Upon feedstock depletion in the third reactor vessel, the remaining liquid fraction can be returned to the first reactor vessel, the second reactor vessel 2 or discharged as wastewater.

### Example 3. Food Waste

Feed stock: 1. Vegetable processing facility waste solids; and/or
   2. Process wash and waste water
Process Steps:
   I. Hydrogen
   II. Methane
   III. Hydrogen

Methodology: The microbial inoculant (a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2) is added to an aqueous solution containing one or more of the feedstocks listed and the solution is added to the first reactor vessel. The pH and ORP are allowed to come into range through biological activity utilizing a sealed oxygen impermeable reactor vessel. For example, pH of 6 to 3 for hydrogen production and a pH of 4 to 8.5 for methane production. ORP is reduced by 50 mV from the starting point for hydrogen and will go down as low as −600 mV or more depending on the amount of iron in the feedstock, and above −50 mV for methane production. The ORP for hydrogen production to start is based on a fall of 50 points from starting point, and is not based on a fixed starting point. This allows the hydrogen to be produced and is captured through a piping system attached to the top of the first reactor vessel. The reaction is allowed to continue until all or most the feedstock organic material is converted to organic acids that will lower the pH to the point that the reaction is impaired. All or a portion of the aqueous solution is transferred to a second reactor vessel. This solution is aerated with a gas containing oxygen to raise the dissolved oxygen above 2 mg/L and this oxygenated state of the aqueous solution is maintained until the pH and ORP rises to the desired levels (greater than 4 for pH and greater than −50 mV for ORP preferred in this example). Methane is produced while maintaining a dissolved oxygen greater than 2 mg/L by injecting an oxygenated gas into the second reactor vessel. Then the aqueous solution after methane production slows to a predetermined level can be either transferred back to the first reactor vessel or the third reactor vessel for additional hydrogen creation. This will conserve total water volume and usage in the system. The third reactor vessel is allowed to become anoxic (dissolved oxygen lower than 2 mg/L) and the hydrogen microbes will resume hydrogen production from the remaining organic carbon feedstock. Upon feedstock depletion in the third reactor vessel 3, the remaining liquid fraction can be returned to the first reactor vessel, the second reactor vessel 2, or discharged as wastewater.

### Example 4. Latrine and Septic Tank Waste

Feedstock: 1. Human waste solids and liquids;
   2. Process waste water from food preparation; and/or
   3. Food waste
Process Steps:
   I. Hydrogen
   II. Methane
   III. Hydrogen

Methodology: The microbial inoculant (a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2) is added to an aqueous solution containing one or more of the feedstocks listed above and the solution is added to the first reactor vessel. The pH and ORP are allowed to come into range through biological activity utilizing a sealed oxygen impermeable reactor vessel. For example, pH of 6 to 3 for hydrogen production and a pH of 4 to 8.5 for methane production. ORP is reduced by 50 mV from the starting point for hydrogen and will go down as low as −600 mV or more depending on the amount of iron in the feedstock, and above −50 mV for methane production. The ORP for hydrogen production to start is based on a fall of 50 points from starting point, and is not based on a fixed starting point. This allows the hydrogen to be produced and is captured through a piping system attached to the top of the first reactor vessel. The reaction is allowed to continue until all or most the feedstock organic material is converted to organic acids that will lower the pH to the point that the reaction is impaired. All or a portion of the aqueous solution is transferred to the second reactor vessel. This solution is aerated with a gas containing oxygen to raise the dissolved oxygen above 2 mg/L and this oxygenated state of the aqueous solution is maintained until the pH and ORP rises to the desired levels (greater than 4 for pH and greater than −50 mV for ORP preferred in this example). Methane is produced while maintaining a dissolved oxygen greater than 2 mg/L by injecting an oxygenated gas into the second reactor vessel. Then the aqueous solution after methane production slows to a predetermined level can be either transferred back to the first reactor vessel or the third reactor vessel for additional hydrogen creation. This will conserve total water volume and usage in the system. The third reactor vessel is allowed to become anoxic (dissolved oxygen lower than 2 mg/L) and the hydrogen microbes will resume hydrogen production from the remaining organic carbon feedstock. Upon feedstock depletion in the third reactor vessel, the remaining liquid fraction can be returned to the first reactor vessel, the second reactor vessel, or discharged as wastewater.

### Example 5. Corn Ethanol Plant Conversion

Feedstock: 1. Corn solids;
   2. Waste corn solids (DDGS);
   3. Thin stillage waste water; and/or
   4. Heat from distillation
Process Steps:
   i. Hydrogen
   ii. Methane
   iii. Hydrogen

Methodology: The microbial inoculant (a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2) is added to an aqueous solution containing one or more of the feedstocks listed above and the solution is added to the first reactor vessel. The pH and ORP are allowed to come into range through biological activity utilizing a sealed oxygen impermeable reactor vessel. For example, pH of 6 to 3 for hydrogen production and a pH of 4 to 8.5 for methane production. ORP is reduced by 50 mV from the starting point for hydrogen and will go down as low as −600 mV or more depending on the amount of iron in the feedstock, and above −50 mV for methane production. The ORP for hydrogen production to start is based on a fall of 50 points from starting point, and is not based on a fixed starting point. This allows the hydrogen to be produced and is captured through a piping system attached to the top of the first reactor vessel. The reaction is allowed to continue until all or most the feedstock organic material is converted to organic acids that will lower the pH to the point that the reaction is impaired. All or a portion of the aqueous solution is transferred to a second reactor vessel. This solution is aerated with a gas containing oxygen to raise the dissolved oxygen above 2 mg/L and this oxygenated state of the aqueous solution is maintained until the pH and ORP rises to the desired levels (greater than 4 for pH and greater than −50 mV for ORP preferred in this example). Methane is produced while maintaining a dissolved oxygen greater than 2 mg/L by injecting an oxygenated gas into the second reactor vessel. Then the aqueous solution after methane production slows to a predetermined level can be either transferred back to the first reactor vessel or the third reactor vessel for additional hydrogen creation. This will conserve total water volume and usage in the system. The third reactor vessel is allowed to become anoxic (dissolved oxygen lower than 2 mg/L) and the hydrogen microbes will resume hydrogen production from the remaining organic carbon feedstock. Upon feedstock depletion in the third reactor vessel, the remaining liquid fraction can be returned to the first reactor vessel, the second reactor vessel, or discharged as wastewater.

### Example 6. Biomass Ethanol Plant Conversion

Feedstock: 1. Biomass solids;
   2. Waste biomass solids (post ethanol production);
   3. Thin stillage waste water; and/or
   4. Heat from distillation
Process Steps:
   I. Hydrogen
   II. Methane
   III. Hydrogen

Methodology: The microbial inoculant (a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2) is added to an aqueous solution containing one or more of the feedstocks listed above and the solution is added to the first reactor vessel. The pH and ORP are allowed to come into range through biological activity utilizing a sealed oxygen impermeable reactor vessel. For example, pH of 6 to 3 for hydrogen production and a pH of 4 to 8.5 for methane production. ORP is reduced by 50 mV from the starting point for hydrogen and will go down as low as −600 mV or more depending on the amount of iron in the feedstock, and above −50 mV for methane production. The ORP for hydrogen production to start is based on a fall of 50 points from starting point, and is not based on a fixed starting point. This allows the hydrogen to be produced and is captured through a piping system attached to the top of the first reactor vessel. The reaction is allowed to continue until all or most the feedstock organic material is converted to organic acids that will lower the pH to the point that the reaction is impaired. All or a portion of the aqueous solution is transferred to a second reactor vessel. This solution is aerated with a gas containing oxygen to raise the dissolved oxygen above 2 mg/L and maintains this oxygenated state of the aqueous solution until the pH and ORP rises to the desired levels (greater than 4 for pH and greater than −50 mV for ORP preferred in this example). Methane is produced while maintaining a dissolved oxygen greater than 2 mg/L by injecting an oxygenated gas into the second reactor vessel. Then the aqueous solution after methane production slows to a predetermined level can be either transferred back to the first reactor vessel or the third reactor vessel for additional hydrogen creation. This will conserve total water volume and usage in the system. The third reactor vessel is allowed to become anoxic (dissolved oxygen lower than 2 mg/L) and the hydrogen microbes will resume hydrogen production from the remaining organic carbon feedstock. Upon feedstock depletion in the third reactor vessel, the remaining liquid fraction can be returned to the first reactor vessel, the second reactor vessel, or discharged as wastewater.

### Example 7. Municipal Wastewater

Feedstock: 1. Municipal wastewater solids;
   2. Waste water liquid; and/or
   3. Wastewater microbial biomass
Process Steps:
   I. Hydrogen
   II. Methane
   III. Hydrogen

Methodology: The microbial inoculant (a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2) is added to an aqueous solution containing one or more of the feedstocks listed above and the solution is added to the first reactor vessel. The pH and ORP are allowed to come into range through biological activity utilizing a sealed oxygen impermeable reactor vessel. For example, pH of 6 to 3 for hydrogen production and a pH of 4 to 8.5 for methane production. ORP is reduced by 50 mV from the starting point for hydrogen and will go down as low as −600 mV or more depending on the amount of iron in the feedstock, and above −50 mV for methane production. The ORP for hydrogen production to start is based on a fall of 50 points from starting point, and is not based on a fixed starting point. This allows the hydrogen to be produced and is captured through a piping system attached to the top of the first reactor vessel. The reaction is allowed to continue until all or most the feedstock organic material is converted to organic acids that will lower the pH to the point that the reaction is impaired. All or a portion of the aqueous solution is transferred to a second reactor vessel. This solution is aerated with a gas containing oxygen to raise the dissolved oxygen above 2 mg/L and maintains this oxygenated state of the aqueous solution until the pH and ORP rises to the desired levels (greater than 4 for pH and greater than −50 mV for ORP preferred in this example). Methane is produced while maintaining a dissolved oxygen greater than 2 mg/L by injecting an oxygenated gas into the second reactor vessel. Then the aqueous solution after methane production slows to a predetermined level can be either transferred back to the first reactor vessel or the third reactor vessel for additional hydrogen creation. This will conserve total water volume and usage in the system. The third reactor vessel is allowed to become anoxic (dissolved oxygen lower than 2 mg/L) and the hydrogen microbes will resume hydrogen production from the remaining organic carbon feedstock. Upon feedstock depletion in the third reactor vessel, the remaining liquid fraction can be returned to the first reactor vessel, the second reactor vessel, or discharged as wastewater.

### Example 8. Landfill Utilizing Existing Liner No External Tanks

Feedstock: 1. Landfill liquid leachate; and/or
   2. Landfill solids
Process Steps:
   I. Hydrogen
   II. Methane

Methodology: The microbial inoculant (a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2) is added to an aqueous solution containing one or more of the feedstocks listed above and the solution is added to the sealed landfill using the existing infrastructure to create the sealed environment. The pH and ORP are allowed to come into range through biological activity utilizing the sealed oxygen impermeable landfill infrastructure. For example, pH of 6 to 3 for hydrogen production and a pH of 4 to 8.5 for methane production. ORP is reduced by 50 mV from the starting point for hydrogen and will go down as low as −600 mV or more depending on the amount of iron in the feedstock, and above −50 mV for methane production. The ORP for hydrogen production to start is based on a fall of 50 points from starting point, and is not based on a fixed starting point. This allows the hydrogen to be produced and is captured through a piping system attached to the top of the landfill tank or pit. The reaction is allowed to continue until all or most the feedstock organic material is converted to organic acids that will lower the pH to the point that the reaction is impaired. The leachate solution is pumped from the bottom of the landfill infrastructure which could be a pit or tank system and aerated with a gas containing oxygen to raise the dissolved oxygen above 2 mg/L and this oxygenated state of the aqueous solution is maintained until the pH and ORP rises to the desired levels (greater than 4 for pH and greater than −50 mV for ORP preferred in this example). This aerated solution will be returned to the landfill infrastructure and methane is produced while maintaining a dissolved oxygen greater than 2 mg/L by injecting an oxygenated gas into the infrastructure leachate. Then the aqueous solution after methane production slows to a predetermined level can be either transitioned to an anoxic and low ORP condition for additional hydrogen creation or left in place as depleted leachate.

### Example 9. Landfill Utilizing Leachate with External Tanks

Feedstock: Landfill liquid leachate
Process Steps:
   I. Hydrogen
   II. Methane
   III. Hydrogen

Methodology: The microbial inoculant (a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2) is added to an aqueous solution containing one or more of the feedstocks listed and the solution is added to the first reactor vessel. The pH and ORP are allowed to come into range through biological activity utilizing a sealed oxygen impermeable reactor vessel. For example, pH of 6 to 3 for hydrogen production and a pH of 4 to 8.5 for methane production. ORP is reduced by 50 mV from the starting point for hydrogen and will go down as low as −600 mV or more depending on the amount of iron in the feedstock, and above −50 mV for methane production. The ORP for hydrogen production to start is based on a fall of 50 points from starting point, and is not based on a fixed starting point. This allows the hydrogen to be produced and is captured through a piping system attached to the top of the first reactor vessel. The reaction is allowed to continue until all or most the feedstock organic material is converted to organic acids that will lower the pH to the point that the reaction is impaired. All or a portion of the aqueous solution is transferred to a second reactor vessel. This solution is aerated with a gas containing oxygen to raise the dissolved oxygen above 2 mg/L and this oxygenated state of the aqueous solution is maintained until the pH and ORP rises to the desired levels (greater than 4 for pH and greater than −50 mV for ORP preferred in this example). Methane is produced while maintaining a dissolved oxygen greater than 2 mg/L by injecting an oxygenated gas into the second reactor vessel. Then the aqueous solution after methane production slows to a predetermined level can be either transferred back to the first reactor vessel or the third reactor vessel for additional hydrogen creation. This will conserve total water volume and usage in the system. The third reactor vessel is allowed to become anoxic (dissolved oxygen lower than 2 mg/L) and the hydrogen microbes will resume hydrogen production from the remaining organic carbon feedstock. Upon feedstock depletion in the third reactor vessel, the remaining liquid fraction can be returned to the first reactor vessel, the second reactor vessel, or discharged as wastewater.

What is claimed is:

1. A method for selectively and separately producing hydrogen and methane, the method comprising:
a) contacting a biomass in a first reactor vessel with a first microbial inoculant composition under anaerobic conditions to facilitate the digestion of the biomass to produce a digested biomass, wherein the first reactor vessel is maintained at a first oxidation reduction potential (ORP);
b) collecting hydrogen gas from the first reactor vessel;
c) transferring a portion of digested biomass from step a) to a second reactor vessel;
d) introducing an oxygen-containing gas to the second reactor vessel to change the first ORP from the first reactor vessel to a second ORP in the second reactor vessel and contacting the digested biomass in the second reactor vessel with a second microbial inoculant composition under anaerobic conditions to facilitate the digestion of the digested biomass; and
e) collecting biogas from the second reactor vessel,
wherein the first microbial inoculant comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2.

2. The method of claim 1, wherein the first microbial inoculant composition in step a) decreases or suppresses methanogens in the first reactor vessel.

3. The method of claim 1, wherein the biomass and the first microbial inoculant composition are introduced into the first reactor vessel at the same time.

4. The method of claim 1, wherein the biomass and the second microbial inoculant composition are introduced into the second reactor vessel at the same time.

5. The method of claim 1, wherein step a) comprises maintaining the oxidation reduction potential (ORP) between around −50 mV and −600 mV.

6. The method of claim 1, wherein step c) comprises maintaining the oxidation reduction potential (ORP) between around −100 mV and less than 1000 mV.

7. The method of claim 1, wherein step c) comprises maintaining the oxidation reduction potential (ORP) between around −300 mV and less than −400 mV.

8. The method of claim 1, further comprising maintaining a pH level of the contents of the first reactor vessel at a first pH level or within a first pH range.

9. The method of claim 8, wherein first pH level is less than 6 or the first pH range between 1 and 6.

10. The method of claim 1, further comprising maintaining the temperature of the contents of the first reactor vessel between 97° C. and 106° C.

11. The method of claim 1, wherein the biomass is a feedstock, plant material, an animal material, food, water, industrial waste or organic waste products or residual waste thereof.

12. The method of claim 1, wherein the biomass is pretreated with a microbial inoculant composition comprising a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2.

13. The method of claim 12, wherein the first microbial inoculant comprises one or more of the microbes listed in Table 1, Table 2 or Table 3.

14. The method of claim 12, wherein the first microbial inoculant comprises at least one different microbial strain, wherein the 16S sequence of the one different microbial strain comprises a 16S sequence that is at least about 97% identical to one or more of the 16S sequences listed in Table 1, Table 2 or Table 3.

15. The method of claim 1, further comprising: e) transferring a portion of the digested biomass from step c) to a third reactor vessel and contacting the digested biomass in the third reactor vessel with a third microbial inoculant composition to facilitate the digestion of the biomass under conditions to convert ammonia into nitrates.

16. The method of claim 15, wherein the third microbial inoculant composition comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2.

17. The method of claim 15, wherein the third microbial inoculant composition is the same as the first microbial inoculant composition in step a).

18. The method of claim 15, further comprising comprises maintaining the oxidation reduction potential (ORP) in the third reactor vessel between around −80 mV and −800 mV.

19. The method of claim 15, wherein the biomass in the third reactor vessel is separated into a solid portion and a liquid portion.

20. The method of claim 19, wherein the solid portion of the biomass is separated into primitive carbon(s).

21. The method of claim 19, wherein the liquid portion comprises inorganic plant nutrients.

22. The method of claim 18 or 19, wherein the total amounts of inorganic plant nutrients in the liquid portion are increased.

23. The method of claim 19, further comprising collecting a portion of the liquid portion from the third reactor vessel.

24. The method of claim 19, further comprising collecting a portion of the solid portion from the third reactor vessel.

25. A method for selectively and separately producing hydrogen and methane, the method comprising:
a) contacting a biomass in a first reactor vessel with a first microbial inoculant composition under anaerobic conditions to facilitate the digestion of the biomass to produce a digested biomass, wherein the first reactor vessel is maintained at a first oxidation reduction potential (ORP);
b) collecting hydrogen gas from the first reactor vessel and transferring a portion of the digested biomass from step a) to a second reactor vessel;
c) introducing an oxygen-containing gas to the second reactor vessel to change the first ORP from the first reactor vessel to a second ORP in the second reactor vessel and contacting the digested biomass in the second reactor vessel with a second microbial inoculant composition under aerobic conditions to facilitate the digestion of the digested biomass; and
d) collecting biogas from the second reactor vessel,
e) collecting a portion of the digested biomass from step a) and separating a liquid fraction from a solid fraction of the digested biomass,
f) transferring the solid fraction of step e) into the first or second reactor vessel or both the first and second reactor vessels,
g) transferring the liquid fraction or supernatant of step e) into a moving biofilm bed reactor (MBBR), contacting the liquid fraction in the MBBR with a microbial inoculant composition similar or the same as the content of the microbial inoculant composition used in the second reactor vessel;
h) digesting the liquid fraction in the MBBR under conditions to remove one or more organic acids from the liquid fraction to produce a liquid fraction with a reduced one or more organic acids content; and
i) optionally, transferring the liquid fraction or supernatant with a reduced one or more organic acids content of step h) into the first reactor vessel, wherein the microbial inoculant comprising comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2, wherein the second microbial inoculant comprises one or more methanogen producers.

26. The method of claim 25, wherein the MBBR is maintained at an ORP similar or same ORP as the second ORP in the second reactor vessel.

27. The method of claim 25, further comprising collecting biogas from the MBBR.

28. The method of claim 25, wherein the MBBR comprises a microbial inoculant composition.

29. The method of claim 25, wherein the first microbial inoculant composition in step a) decreases or suppresses methanogens in the first reactor vessel.

30. The method of claim 25, wherein the biomass and first microbial inoculant composition are introduced into the first reactor vessel at the same time.

31. The method of claim 25, wherein the biomass and the second microbial inoculant composition are introduced into the second reactor vessel at the same time.

32. The method of claim 25, wherein step a) comprises maintaining the oxidation reduction potential (ORP) between around −50 mV and −600 mV.

33. The method of claim 25, wherein step c) comprises maintaining the oxidation reduction potential (ORP) between around −100 mV and less than 1000 mV.

34. The method of claim 25, wherein step c) comprises maintaining the oxidation reduction potential (ORP) between around −300 mV and less than −400 mV.

35. The method of claim 25, further comprising maintaining a pH level of the contents of the first reactor vessel at a first pH level or within a first pH range.

36. The method of claim 35, wherein first pH level is less than 6 or the first pH range between 1 and 6.

37. The method of claim 25, further comprising maintaining the temperature of the contents of the first reactor vessel between 97° C. and 106° C.

38. The method of claim 25, wherein the biomass is a feedstock, plant material, an animal material, food, water, industrial waste or organic waste products or residual waste thereof.

39. The method of claim 25, wherein the biomass is pretreated with a microbial inoculant composition comprising a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2.

40. The method of claim 39, wherein the first microbial inoculant comprises one or more of the microbes listed in Table 1, Table 2 or Table 3.

41. The method of claim 39, wherein the first microbial inoculant comprises at least one different microbial strain, wherein the 16S sequence of the one different microbial strain comprises a 16S sequence that is at least about 97% identical to one or more of the 16S sequences listed in Table 1, Table 2 or Table 3.

42. The method of claim 25, further comprising: transferring a portion of the digested biomass from the first or second reactor to a third reactor vessel and contacting the digested biomass in the third reactor vessel with a third microbial inoculant composition to facilitate the digestion of the biomass under conditions to convert ammonia into nitrates.

43. The method of claim 42, wherein the third microbial inoculant composition comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2.

44. The method of claim 25, wherein the third microbial inoculant composition is the same as the first microbial inoculant composition in step a).

45. The method of claim 25, further comprising collecting liquids from the third reactor vessel.

46. The method of claim 25, further comprising comprises maintaining the oxidation reduction potential (ORP) in the third reactor vessel between around −80 mV and −800 mV.

47. The method of claim 25, wherein the biomass in the third reactor vessel is separated into a solid portion and a liquid portion.

48. The method of claim 47, wherein the solid portion of the biomass is separated into primitive carbon(s).

49. The method of claim 47, wherein the liquid portion comprises inorganic plant nutrients.

50. The method of claim 48 or 49, wherein the total amounts of inorganic plant nutrients in the liquid portion are increased.

51. The method of claim 25, further comprising collecting a portion of the liquid portion from the third reactor vessel.

52. The method of claim 25, further comprising collecting a portion of the solid portion from the third reactor vessel.

53. A method for selectively producing hydrogen from a landfill leachate, the method comprising the steps of:
a) applying a composition comprising two or more bacterial strains, wherein a first bacterial strain comprises Clostridium spp., and wherein the 16S sequence of Clostridium spp. comprises any one of the Clostridium spp. listed in Table 1 or Table 2 and a second bacterial strain comprising an aquatic Pseudomonas spp. bacteria with a 16S nucleic acid sequence that is at least about 97% identical to any one of the Pseudomonas spp. bacteria listed in Table 1 or Table 2 to the landfill leachate;
b) collecting samples from the landfill leachate;
c) introducing the landfill leachate sample into a first reactor vessel and contacting the landfill leachate sample with the microbial inoculant composition in step a) under anaerobic conditions to facilitate the digestion of the landfill leachate sample wherein the first reactor vessel is maintained at a first oxidation reduction potential (ORP);
d) collecting hydrogen gas from the first reactor vessel and transferring a portion of the digested landfill leachate sample from step c) to a second reactor vessel;
e) introducing an oxygen-containing gas to the second reactor vessel to change the first ORP from the first reactor vessel to a second ORP in the second reactor vessel and contacting the digested landfill leachate sample in the second reactor vessel with a second microbial inoculant composition under aerobic conditions to facilitate the digestion of the landfill leachate sample; and
f) collecting biogas from the second reactor vessel,
g) collecting a portion of the digested landfill leachate sample from step c) and separating a liquid fraction from a solid fraction of the portion of the digested landfill leachate sample,
h) transferring a portion of the solid fraction of step g) into the first or second reactor vessel or both the first and second reactor vessels,
i) transferring the liquid fraction or supernatant of step g) into a moving biofilm bed reactor (MBBR), contacting the liquid fraction in the MBBR with a microbial inoculant composition similar or the same as the content of the microbial inoculant composition used in the second reactor vessel;
j) digesting the liquid fraction in the MBBR under conditions to remove one or more organic acids from the liquid fraction to produce a liquid fraction with a reduced one or more organic acids content,
k) optionally, transferring the liquid fraction or supernatant with a reduced one or more organic acids content of step j) into the first reactor vessel, wherein the first microbial inoculant comprising comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2,
wherein the second microbial inoculant comprises one or more methanogens selected from the group of consisting of Methanobacterium bryantii, Methanobacterium formicum, Methanobrevibacter arboriphilicus, Methanobrevibacter gottschalkii, Methanobrevibacter ruminantium, Methanobrevibacter smithii, Methanococcus chunghsingensis, Methanococcus burtonii, Methanococcus aeolicus, Methanococcus deltae, Methanococcus jannaschii, Methanococcus maripaludis, Methanococcus vannielii, Methanocorpusculum labreanum, Methanoculleus bourgensis (Methanogenium olentangyi and Methanogenium bourgense), Methanoculleus marisnigri, Methanoflorens stordalenmirensis, Methanofollis liminatans, Methanogenium cariaci, Methanogenium frigidum, Methanogenium organophilum, Methanogenium wolfei, Methanomicrobium mobile, Methanopyrus kandleri, Methanoregula boonei, Methanosaeta concilii, Methanosaeta thermophile, Methanosarcina acetivorans, Methanosarcina barkeri, Methanosarcina mazei, Methanosphaera stadtmanae, Methanospirillium hungatei, Methanothermobacter defluvii (Methanobacterium defluvii), Methanothermobacter thermautotrophicus (Methanobacterium thermoautotrophicum), Methanothermobacter thermoflexus (Methanobacterium thermoflexum), Methanothermobacter wolfei (Methanobacterium wolfei), and Methanothrix sochngenii.

54. The method of claim 53, wherein the MBBR is maintained at an ORP similar or same ORP as the second ORP in the second reactor vessel.

55. The method of claim 53, further comprising collecting biogas from the MBBR.

56. The method of claim 53, wherein the landfill leachate comprises a plant material, an animal material, food water, industrial waste or organic waste products or residual waste thereof.

57. The method of claim 53, wherein the MBBR comprises a microbial inoculant composition.

58. The method of claim 53, wherein the first microbial inoculant composition in step a) decreases or suppresses methanogens in the first reactor vessel.

59. The method of claim 53, wherein the landfill leachate and first microbial inoculant composition are introduced into the first reactor vessel at the same time.

60. The method of claim 53, wherein the landfill leachate and the second microbial inoculant composition are introduced into the second reactor vessel at the same time.

61. The method of claim 53, wherein step a) comprises maintaining the oxidation reduction potential (ORP) between around −50 mV and −600 mV.

62. The method of claim 53, wherein step c) comprises maintaining the oxidation reduction potential (ORP) between around −100 mV and less than 1000 mV.

63. The method of claim 53, wherein step c) comprises maintaining the oxidation reduction potential (ORP) between around −300 mV and less than −400 mV.

64. The method of claim 53, further comprising maintaining a pH level of the contents of the first reactor vessel at a first pH level or within a first pH range.

65. The method of claim 64, wherein first pH level is less than 6 or the first pH range between 1 and 6.

66. The method of claim 53, further comprising maintaining the temperature of the contents of the first reactor vessel between 97° C. and 106° C.

67. The method of claim 53, wherein the landfill leachate comprises feedstock, plant material, an animal material, food, water, industrial waste or organic waste products or residual waste thereof.

68. The method of claim 53, wherein the landfill leachate is pretreated with a microbial inoculant composition comprising a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2.

69. The method of claim 53, wherein the first microbial inoculant comprises one or more of the microbes listed in Table 1, Table 2 or Table 3.

70. The method of claim 53, wherein the first microbial inoculant comprises at least one different microbial strain, wherein the 16S sequence of the one different microbial strain comprises a 16S sequence that is at least about 97% identical to one or more of the 16S sequences listed in Table 1, Table 2 or Table 3.

71. The method of claim 53, further comprising: transferring a portion of the digested landfill leachate from the first or second reactor to a third reactor vessel and contacting the digested landfill leachate in the third reactor vessel with a third microbial inoculant composition to facilitate the digestion of the landfill leachate under conditions to convert ammonia into nitrates.

72. The method of claim 71, wherein the third microbial inoculant composition comprises a first bacterial strain and a second bacterial strain, wherein the first bacterial strain comprises Clostridium spp., and wherein the 16S sequence of Clostridium spp. comprises any one of the Clostridium spp. listed in Table 1 or Table 2 and the second bacterial strain comprises an aquatic Pseudomonas spp. bacteria with a 16S nucleic acid sequence that is at least about 97% identical to any one of the Pseudomonas spp. bacteria listed in Table 1 or Table 2.

73. The method of claim 71, wherein the third microbial inoculant composition is the same as the first microbial inoculant composition in step a).

74. The method of claim 71, further comprising collecting liquids from the third reactor vessel.

75. The method of claim 53, further comprising comprises maintaining the oxidation reduction potential (ORP) in the third reactor vessel between around −80 mV and −800 mV.

76. The method of claim 53, wherein the landfill leachate in the third reactor vessel is separated into a solid portion and a liquid portion.

77. The method of claim 53, wherein the solid portion of the landfill leachate is separated into primitive carbon(s).

78. The method of claim 76, wherein the liquid portion comprises inorganic plant nutrients.

79. The method of claim 76 or 77, wherein the total amounts of inorganic plant nutrients in the liquid portion are increased.

80. The method of claim 53, further comprising collecting a portion of the liquid portion from the third reactor vessel.

81. The method of claim 53, further comprising collecting a portion of the solid portion from the third reactor vessel.
